(19) United States
(12) Patent Application Publication
(10) Pub. No.: US 2023/0266317 A1
(43) Pub. Date: Aug. 24, 2023

(54) ANTIGEN BINDING PROTEIN AND ASSAYS

(71) Applicant: GLAXOSMITHKLINE BIOLOGICALS SA, Rixensart (BE)

(72) Inventors: Michael CHAPLET, Rixensart (BE); Nathalie NORAIS, Siena (IT); Simona RONDINI, Siena (IT); Silvia ROSSI PACCANI, Siena (IT)

(73) Assignee: GLAXOSMITHKLINE BIOLOGICALS SA, Rixensart (BE)

(21) Appl. No.: 17/641,315

(22) PCT Filed: Sep. 9, 2020
(86) PCT No.: PCT/EP2020/075128

§ 371 Date: Mar. 8, 2022

(30) Foreign Application Priority Data
Sep. 11, 2019 (EP) 19196718.1

(51) Int. Cl. G01N 33/569; C07K 16/12

(52) U.S. Cl. CPC G01N 33/56911; C07K 16/1203; G01N 2333/212; C07K 2317/33

(57) ABSTRACT

The present invention relates to in vitro assays, more particularly ELISA assays. Said ELISA assays comprise antibodies capable of binding Ubiquitous surface protein A2 (UspA2) from Moraxella catarrhalis. The present invention relates to assays for assessing the binding of antibodies to UspA2 and the relative potency of vaccine test samples comprising UspA2. In particular, the invention relates to in vitro relative potency assays used in the release of vaccine that comprises UspA2 to the public.

### TECHNICAL FIELD

The present invention relates to the field of vaccines, in particular the quality assurance processes involved in the release of manufactured vaccines to the public. More particularly, the present invention relates to an in vitro assay involving antibodies which bind to Ubiquitous surface protein A2 (UspA2) from Moraxella catarrhalis. As such, the present invention relates to an in vitro relative potency (IVRP) assay for use during batch release of a vaccine comprising the UspA2 antigen.

### BACKGROUND

Ubiquitous surface protein A2 (UspA2) from Moraxella catarrhalis is a trimeric autotransporter that appears as a lollipop-shared structure in electron micrographs. It is composed of an N-terminal head, followed by a stalk, which ends by an amphipathic helix and a C-terminal membrane domain (Hoiczyk E et al. EMBO J. 2000; 19(22):5989-99). UspA2 contains a very well conserved domain (Aebi C et al. Infect Immun. 1997; 65(11):4367-77) which is recognized by a monoclonal antibody (17C7) that was shown protective upon passive transfer in a mouse Moraxella catarrhalis challenge model (Helminen M E, et al. J Infect Dis. 1994; 170(4):867-72). UspA2 can be distinguished from Ubiquitous surface protein A1 (UspA1) by differences in amino acid sequences within the head and membrane-spanning regions, yet they share homology within the stalk region. UspA2H is a “hybrid” protein containing a head region (N-terminal) similar to that of UspA1 while having the UspA2-like C-terminal region.

UspA2 is heat modifiable with a predicted molecular weight of 60 kDa, but it appears above 200 kDa after denaturation in SDS-PAGE (Cope L D et al. J Bacteriol. 1999; 181(13):4026-34). UspA2 has been shown to interact with host structures and extracellular matrix proteins like fibronectin (Tan T T et al. J Infect Dis. 2005; 192(6):1029-38) and laminin (Tan T T et al. J Infect Dis. 2006; 194(4):493-7) suggesting it can play a role at an early stage of Moraxella catarrhalis infection.

UspA2 also seems to be involved in the ability of Moraxella catarrhalis to resist the bactericidal activity of normal human serum (Attia A S et al. Infect Immun. 2005; 73(4):2400-10). It (i) binds the complement inhibitor C4 bp, enabling Moraxella catarrhalis to inhibit the classical complement system, (ii) prevents activation of the alternative complement pathway by absorbing C3 from serum and (iii) interferes with the terminal stages of the complement system, the Membrane Attack Complex (MAC), by binding the complement regulator protein vitronectin (de Vries et al. Microbiol Mol Biol Rev. 2009; 73(3):389-406).

Moraxella catarrhalis is an important and common respiratory pathogen that has been associated with increased risk of exacerbations in chronic obstructive pulmonary disease (COPD) in adults (Perez A C, Murphy T F. Potential impact of a Moraxella catarrhalis vaccine in COPD. Vaccine. 2017). COPD is a leading cause of morbidity and mortality worldwide. A common preventable disease, COPD is characterised by persistent airflow limitation that is usually progressive. The airflow limitation is associated with an enhanced chronic inflammatory response in the airways and lungs to noxious particles of gases. It is a multi-component disease that manifests as an accelerated decline in lung function, with symptoms such as breathlessness on physical exertion, deteriorating health status and exacerbations.

Acute exacerbations and comorbidities contribute to the overall disease severity in individual COPD patients. An acute exacerbation of COPD (AECOPD) is an acute event characterised by a worsening of the patient's respiratory symptoms that is beyond normal day-to-day variations and leads to a change in medication [Perez A C, Murphy T F. Potential impact of a Moraxella catarrhalis vaccine in COPD. Vaccine. 2017]. AECOPD increases morbidity and mortality, leading to faster decline in lung function and poorer functional status [Sapey E, Stockley R A. COPD exacerbations. 2: aetiology. Thorax. 2006; 61 (3):250-8)]. The lungs are known to be colonised with different species of bacteria [Erb-Downward J R, et al. PLoS One. 2011; 6(2):e]6384 and Wilkinson TMA, et al. Thorax. 2017; 72(10):919-27]. In COPD patients, acquisition of new bacterial strains is believed to be an important cause of AECOPD [Sethi S, et al. N Engl J Med. 2002; 347(7):465-71]. Although estimates vary widely, Non-Typeable Haemophilus influenzae (NTHi) appears to be the main bacterial pathogen associated with AECOPD (11-38%), followed by Moraxella catarrhalis (3-25%) and Streptococcus pneumoniae (4-9%) [Alamoudi O S. et al. Respirology. 2007; 12(2):283-7, Bandi V, et al. FEMS Immunol Med Microbiol. 2003; 37(1):69-75, Beasley V, et al. Int J Chron Obstruct Pulmon Dis. 2012; 7:555-69]. A vaccine against AECOPD is in clinical development.

Vaccines normally require the manufacturer to test each batch prior to its release for public use. It is desirable to provide an in vitro test since historically in vivo release assays were used which require immunization of many animals. Furthermore, in vitro assays are more sensitive (in terms of detecting marginal effects on vaccine batches) than in vivo studies. Suitable assessments may include potency, structure or immunogenicity. Suitably, such in vitro assay could be used to confirm that a particular batch of vaccine will be expected to have in vivo activity in human recipients. Therefore, there is a need to provide an in vitro assay for assessing the potency of vaccines containing UspA2.

### SUMMARY OF THE INVENTION

The present invention provides antibodies which bind to UspA2. The present invention also relates to assays (particularly in vitro assays) for assessing binding to UspA2 and the potency of vaccines containing UspA2. The assays use antibodies which bind to UspA2, in particular antibodies which are functional, and/or which recognise epitopes within the UspA2 protein. By comparing the results of a test sample that comprises UspA2 with those obtained using a standard or reference sample of known potency, it is possible to determine the relative potency of the test sample. This can be used for determining whether a manufactured batch of a vaccine is suitable for release to the public, or whether it has experienced a production failure and so should not be used.

Accordingly, in a first aspect of the invention there is provided the use of an antibody which binds to UspA2 in an assay for the detection of, or measurement of a change in, the conformation of UspA2 wherein UspA2 is present in a test sample.

In a further aspect of the invention there is provided, an assay comprising the steps of contacting a test sample comprising UspA2 with a first antibody and a second antibody to form a first antibody-UspA2-second antibody complex and detecting or measuring the amount of said first antibody-UspA2-second antibody complex.

In a further aspect of the invention there is provided a kit to (i) detect, measure the levels of, and/or measure a change in the conformation of a test antigen or (ii) determine potency of a test antigen, comprising: reagents for preparing an assay mixture, at least one antibody which binds to UspA2, and optionally instructions for use thereof.

In a further aspect of the invention there is provided a method for in vitro analysis of a test antigen, comprising steps of: (i) performing the assay of the invention on a test antigen and a reference sample of known potency; and (ii) comparing the results from step (i) to determine the potency of the test antigen relative to the reference sample.

In a further aspect of the invention there is provided a method for analysing a batch of vaccine, comprising steps of: (i) assaying a test antigen taken from a batch of vaccine by the method of the invention; and, if the results of step (i) indicate an acceptable relative potency, (ii) releasing vaccine from the batch for in vivo use.

### DESCRIPTION OF FIGURES

FIG. 1: Peptide map of UspA2 used for the identification of the epitope recognised by mAb FHUSPA2/10. UspA2 was digested with pepsin and the resulting peptides were analysed by MS/MS. The 132 peptides identified are represented with black lines. Peptides with repeated sequences, in particular peptides 294-329 and 329-363 as well as peptides 230-240 and 395-405 are represented once. Taking into consideration those peptides, the overall sequence coverage is 97.4%.

FIG. 2: Epitope mapping of mAb FHUSPA2/10 used at a molar ratio of UspA2 monomer/antibody of 1/0.33. Relative fractional uptake maps of UspA2 alone or upon binding with mAB (FHUSPA2/10 mAB). 94 peptides among the 132 identified by PLGS were considered for the analysis and are reported along the x-axis. The three peptides relevant for the identification of the epitopes are labelled in FIG. 3, FIG. 4 and FIG. 5 (see below).

FIG. 3: Kinetics of deuterium uptake over 24 hours of peptide Y279-E292 in the absence (black circles) or presence (black squares) of the mAb.

FIG. 4: Kinetics of deuterium uptake over 24 hours of peptide Y279-L297 (SEQ ID NO: 119) in the absence (black circles) or presence (black squares) of the mAb.

FIG. 5: Kinetics of deuterium uptake over 24 hours of peptide A293-D310 (SEQ ID NO: 120) in the absence (black circles) or presence (black squares) of the mAb.

FIG. 6: Epitope mapping of mAb FHUSPA2/10 used at a molar ratio UspA2 monomer/antibody of 1:1. Relative fractional uptake maps of UspA2 alone or upon binding with mAb FHUSPA2/10. 87 peptides among the 132 identified by PLGS were considered for this analysis and are reported along the x-axis. The peptides showing the higher extent of deuterium uptake difference are labelled (Y279-E292, Y279-L297 and Q318-L332/Q353-L367 (SEQ ID NO: 121)).

FIG. 7: Peptide map of UspA2 used for the identification of the epitope recognised by the UspA2 rabbit-pAb. UspA2 was digested with pepsin and the resulting peptides were analysed by MS/MS. The 177 peptides identified are represented with black lines. Peptides with repeated sequences are represented once only. The overall sequence coverage was 98.5%.

FIG. 8: Epitope mapping of the rabbit pAb—The relative fractional update maps of UspA2 alone or upon binding with the pAb are reported. The 68 peptides among the 177 identified by PLGS were considered for this analysis reported along the x-axis. The peptides covering the sequence A471-L522 (of SEQ ID NO: 75) showed the higher extent of protection from deuterium exchange as evidenced in the positive y-axis. Peptides covering the sequence L58-N201 showed destabilization upon the binding with pAb as evidenced in the negative y-axis.

FIG. 9: A zoomed view of the “heat map” representation is reported for sequence A471-L522. The scale indicates the level of reduction of deuterium incorporation when the antigen is incubated with the pAb. The darker the underlined sequence, the higher the extent of protection by the antibody. The sequence underlined in black indicates UspA2 residues showing strongest interaction with the pAb—Data not corrected for back exchange.

FIG. 10: The spectra of the peptide 177-197 (charge state+3) derived from the protein denatured alone (left panel) or with the pAb (right panel).

FIG. 11: (A) Sequence alignment between UspA2 (query sequence) and YadA (subject sequence) translocation domains retrieved by BLAST. Query 50 (SEQ ID NO: 122), Subject 17 (SEQ ID NO: 123), Query 110 (SEQ ID NO: 124) and Subject 77 (SEQ ID NO: 125). (B) Ribbon representation of translocation unit model of UspA2. The epitope recognized by the pAb is evidenced. The core epitope D509-F515 is represented in darker grey compare to the adjacent residues 516D-L521 which are also part of the epitope.

FIG. 12: Negative-staining transmission electron microscope (NS-TEM) image of recombinant UspA2 protein in complex with total rabbit IgG. IgG recognizing epitope(s) positions at C-terminal of the stalk (black circle). Very frequently two UspA2 copies were observed positioned in a tail-tail orientation and connected by two IgG copies through the C-terminal epitopes (black Square). Representative images are reported.

FIG. 13: The position of the peptides containing the epitopes is reported for 8 UspA2 variants representative of the variability among circulating M. catarrhalis strains.

FIG. 14: UspA2 IVRP assay showing reduction in potency of UspA2 samples thermally stressed at 45° C. for up to 4 hours (240 minutes). Lines represent experimental repeats (n=5).

### DETAILED DESCRIPTION

### Terminology

To facilitate review of the various embodiments of this disclosure, the following explanations of terms are provided. Additional terms and explanations are provided in the context of this disclosure.

Unless otherwise explained or defined herein, all technical and scientific terms used herein have the same meaning as commonly understood by one of ordinary skill in the art to which this disclosure belongs. For example, definitions of common terms in molecular biology can be found in Benjamin Lewin, Genes V, published by Oxford University Press, 1994 (ISBN 0-19-854287-9); Kendrew et al. (eds.), The Encyclopaedia of Molecular Biology, published by Blackwell Science Ltd., 1994 (ISBN 0-632-02182-9); and Robert A. Meyers (ed.), Molecular Biology and Biotechnology: A Comprehensive Desk Reference, published by VCH Publishers, Inc., 1995 (ISBN 1-56081-569-8).

All references or patent applications cited within this patent specification are incorporated by reference herein.

Amino acids refers to an amino acid selected from the group consisting of alanine (ala, A), arginine (arg, R), asparagine (asn, N), aspartic acid (asp,D), cysteine (cys, C),glutamine (gln, Q), glutamic acid (glu, E), glycine (gly, G), histidine (his, H), isoleucine (ile,I), leucine (leu, L), lysine (lys, K), methionine (met, M), phenylalanine (phe, F), proline (pro, P), serine (ser, S), threonine (thr, T), tryptophan (trp, W), tyrosine (tyr, Y), valine (val, V).

Although methods and materials similar or equivalent to those described herein can be used in the practice or testing of this disclosure, suitable methods and materials are described below.

The abbreviation, “e.g.” is derived from the Latin exempli gratia and is used herein to indicate a non-limiting example. Thus, the abbreviation “e.g.” is synonymous with the term “for example.”

As used herein, the term “epitope” refers to the portion of a macromolecule (antigen) which is specifically recognised by a component of the immune system e.g. an antibody or a T-cell antigen receptor. The term epitope may refer to that portion of the antigen that makes contact with a particular binding domain of the antigen binding protein. An epitope may be linear or conformational/discontinuous. Particular residues comprised within an epitope can be determined through computer modelling programs or via three-dimensional structures obtained through methods known in the art, such as X-ray crystallography. An epitope may reside within the consensus sequence of the invention.

A “subject” as used herein is a mammal, including humans, non-human primates, and non-primate mammals. In one aspect, a subject is a human.

As used herein, “immune response” means the sequence of events occurring at the molecular, cellular or tissue level (i.e. at any level of biological organisation) in response to an antigen. In the context of the present disclosure, “immune response” may be the sequence of cellular (cell mediated) and/or humoral (antibody mediated) events occurring in response to an antigen (e.g. antigens on the surface of bacteria, viruses, fungi etc. or in response to antigens presented in the form of an immunogenic fragment, immunogenic composition or vaccine).

As used herein, “immunogenicity” means the ability of an antigen to elicit an immune response.

As used herein, “adjuvant” means a compound or substance (or combination of compounds or substances) that, when administered to a subject in conjunction with an antigen or antigens, for example as part of an immunogenic composition or vaccine, increases or enhances the subject's immune response to the administered antigen or antigens (compared to the immune response obtained in the absence of adjuvant).

As used herein the term “protect” in the context of infection, diseases or conditions caused by M. catarrhalis means to protect via prophylaxis. Prevention may for example relate to a reduction in the incidence of an infection, disease or condition caused by M. catarrhalis or a reduction in the number of hospitalizations required because of an infection, disease or condition caused by M. catarrhalis. For the purposes of this invention, “prevention of exacerbations of COPD” or “or prevention of AECOPD” refers to a reduction in incidence or rate of COPD exacerbations (for instance a reduction in rate of 0.1, 0.5, 1, 2, 5, 10, 20% or more) or a reduction in severity of COPD exacerbations (e.g. airflow obstruction, chronic bronchitis, bronchiolitis or small airways disease and emphysema), for instance within a patient treatment group immunized with the antibodies, immunogenic compositions or vaccines of the invention.

As used herein the term “treat” in the context of infection, diseases or conditions caused by M. catarrhalis means to treat via administration, post-infection any M. catarrhalis causing symptom, effect or phenotype. Treatment of an infection, disease or condition caused by M catarrhalis includes ameliorating, stabilising, reducing or eliminating the increased symptoms, effects or phenotypes caused by M. catarrhalis in humans.

As used herein the term “amino acid modification” relates to any modification which alters the amino acid sequence of a polypeptide. Modifications may include (but are not limited to) polymorphisms, DNA mutations (including single nucleotide polymorphisms), post-translational modifications etc. Modifications include additions/insertions, deletions, point mutations, substitutions etc. Amino acid substitutions may be conservative or non-conservative. In some embodiments, amino acid substitution is conservative. Substitutions, deletions, additions or any combination thereof may be combined in a single variant so long as the variant is an immunogenic polypeptide. Modifications to the amino acid sequence of a polypeptide may be introduced to the DNA, RNA or protein.

As used herein, the term “conservative amino acid substitution” involves substitution of a native amino acid residue with a non-native residue such that there is little or no effect on the size, polarity, charge, hydrophobicity, or hydrophilicity of the amino acid residue at that position, and without resulting in decreased immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics like those of a parental polypeptide.

Embodiments herein relating to “vaccine compositions” of the invention are also applicable to embodiments relating to “immunogenic compositions” of the invention, and vice versa.

As used herein, the term “deletion” is the removal of one or more amino acid residues from the protein sequence. Typically, no more than about from 1 to 6 residues (e.g. 1 to 4 residues) are deleted at any one site within the protein molecule.

As used herein, the term “insertion” is the addition of one or more non-native amino acid residues in the protein sequence. Typically, no more than about from 1 to 10 residues, (e.g. 1 to 7 residues, 1 to 6 residues, or 1 to 4 residues) are inserted at any one site within the protein molecule.

As used herein “signal peptide” refers to a short (less than 60 amino acids, for example, 3 to 60 amino acids) polypeptide present on precursor proteins (typically at the N terminus), and which is typically absent from the mature protein. The signal peptide (sp) is typically rich in hydrophobic amino acids. The signal peptide directs the transport and/or secretion of the translated protein through the membrane. Signal peptides may also be called targeting signals, transit peptides, localization signals, or signal sequences. For example, the signal sequence may be a co-translational or post-translational signal peptide.

As used herein the term “antigen binding protein” refers to antibodies and other protein constructs, such as domains, which are capable of binding to an antigen (for example UspA2).

As used herein the term “antibody” is used in the broadest sense to refer to molecules with an immunoglobulin-like domain (for example IgG, IgM, IgA, IgD or IgE) and includes monoclonal (mAb), recombinant, polyclonal (pAb), chimeric, human, humanised, multispecific antibodies, including bispecific antibodies, and heteroconjugate antibodies; a single variable domain (e.g., VH, VHH, VL, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing (for a summary of alternative “antibody” formats see [Holliger P, Hudson P J. Engineered antibody fragments and the rise of single domains. Nat Biotechnol. 2005; 23(9):1126-36]). Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer or an EGF domain. In one aspect the antibody is a monoclonal antibody (mAb). In one aspect the antibody is a polyclonal antibody (pAb).

As used herein the term “potency” relates to a measure of biological activity using a suitably quantitative biological assay (also called a potency assay or bioassay), based on the attribute of the product which is linked to the relevant biological properties. A relevant, validated potency assay should be part of the specifications for a biotechnological or biological drug substance and/or drug product. Potency is thus the ability of a biologic to exert its desired effect in patients. It will be acknowledged by those of skill in the art however that “potency” in terms of a vaccine potency assay may be a measure which estimates/predicts whether the biologic will elicit the desired effect in patients and such an assay may be used in releasing a vaccine lot to the market. As such “potency” is a relative term, since potency may be determined by reference to a reference standard or an internal standard. The goal of measuring potency in a release assay format is to ensure lot-to-lot (otherwise termed batch-to-batch) consistency.

As used herein the term “test sample comprising UspA2 or vaccine sample comprising UspA2” relates to an UspA2 antigen which has been manufactured for use in a vaccine and that, prior to release to the public, is to be tested in the in vitro potency assay of the present invention. The test sample comprising UspA2 may be a test sample of the UspA2 protein construct MC-009. The test sample comprising UspA2 may be diluted prior to use in the IVRP assay of the invention. The IVRP assay of the invention aims to detect test samples/vaccine samples comprising UspA2 wherein the UspA2 protein antigen is denatured or has been modified in some way, such that it is sub-optimal for release to the public or such that it does not reach the required threshold of potency compared to the reference sample. The test sample comprising UspA2 may further comprise additional pharmaceutically acceptable excipient(s). Possible excipients include diluents such as water, saline, glycerol etc. The test sample comprising UspA2 may be the final vaccine formulation and may be lyophilized. Additionally, auxiliary substances, such as wetting or emulsifying agents, pH buffering substances, polyols and the like may be present. For example, the test sample comprising UspA2 may comprise water for injection (WFI). In addition, the test sample comprising UspA2 may further comprise additional antigens, for example additional antigens from M. catarrhalis, H. influenzae and/or S. pneumoniae.

As used herein the term “reference sample or reference vaccine sample” relates to an UspA2 antigen which has demonstrated clinical efficacy in humans and is therefore used as a reference standard in the in vitro relative potency assay of the invention. The reference sample may be a quality-assured sample of the UspA2 protein construct MC-009 (SEQ ID NO: 75) for example. The data generated using test samples comprising UspA2 is compared to the reference sample in order to provide a relative assessment of potency (i.e. versus the data generated with the reference sample).

Identity between polypeptides may be calculated by various algorithms. For example, the Needle program, from the EMBOSS package (Free software; EMBOSS: The European Molecular Biology Open Software Suite (2000). Trends in Genetics 16(6): 276-277) and the Gap program from the GCG® package (Accelrys Inc.) may be used. This Gap program is an implementation of the Needleman-Wunsch algorithm described in: [Needleman, S. B. and Wunsch, C. D. (1970) J. Mol. Biol. 48, 443-453]. The BLOSUM62 scoring matrix can be used, and the gap open and extension penalties were respectively 8 and 2. Identity between two polypeptides is calculated across the entire length of both sequences and is expressed as a percentage of the reference sequence.

### UspA2

As used herein “UspA2” means Ubiquitous surface protein A2 from Moraxella catarrhalis. UspA2 may consist of or comprise the amino acid sequence of SEQ ID NO: 1 (UspA2 from ATCC 25238) as well as sequences with at least or exactly 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8% 99.9% or 100% identity, over the entire length, to SEQ ID NO: 1. UspA2 of SEQ ID NO: 1 is encoded by the DNA sequence of SEQ ID NO: 80.

Alternatively, UspA2 may consist of or comprise any of amino acid sequences SEQ ID NO: 1 SEQ ID NO: 46. UspA2 may furthermore consist of or comprise UspA2H and UspA2V variants such as SEQ ID NO: 49-52.

UspA2 as described in SEQ ID NO: 1 contains a signal peptide (for example, amino acids 1 to 29 of SEQ ID NO: 1), a laminin binding domain (for example, amino acids 30 to 177 of SEQ ID NO: 1), a fibronectin binding domain (for example, amino acids 165 to 318 of SEQ ID NO: 1), a C3 binding domain (for example, amino acids 30 to 539 of SEQ ID NO: 1 of WO2007/018463) or a fragment of amino acids 30 to 539 of SEQ ID NO: 1, for example, amino acids 165 to 318 of SEQ ID NO: 1, an amphipathic helix (for example, amino acids 519 to 564 of SEQ ID NO: 1 or amino acids 520-559 of SEQ ID NO:1, identified using different prediction methods) and a C terminal anchor domain (for example, amino acids 576 to 630 amino acids of SEQ ID NO: 1) [see Brooks M J, Sedillo J L, Wagner N, Laurence C A, Wang W, Attia A S, et al. Modular arrangement of allelic variants explains the divergence in Moraxella catarrhalis UspA protein function. Infect Immun. 2008; 76(11):5330-40].

UspA2 amino acid differences have been described for various Moraxella catarrhalis species. Furthermore, both conserved regions and regions of significant amino acid diversity have been reported across M. catarrhalis strains. See for example [Cope et al. J Bacteriol. 1999; 18](13):4026-34, Brooks et al. Infect Immun. 2008; 76(11):5330-40 and Hill D J, Whittles C, Virji M PLoS One. 2012; 7(9):e45452].

UspA2 may consist of or comprise an amino acid sequence that differs from SEQ ID NO. 1 at any one or more amino acid selected from the group consisting of: AA (amino acid) 30 to 298, AA 299 to 302, AA 303 to 333, AA 334 to 339, AA 349, AA 352 to 354, AA 368 to 403, AA 441, AA 451 to 471, AA 472, AA474 to 483, AA 487, AA 490, AA 493, AA 529, AA 532 or AA 543. UspA2 may consist of or comprise an amino acid sequence that differs from SEQ ID NO: 1 in that it contains at least one amino acid insertion in comparison to SEQ ID NO. 1. UspA2 may consist of or comprise an amino acid sequence that differs from SEQ ID NO. 1 at any one of the amino acid differences in SEQ ID NO: 2 through SEQ ID NO: 46. For example, SEQ ID NO. 1 may contain K instead of Q at amino acid 70, Q instead of G at amino acid 135 and/or D instead of N at amino acid 216.

UspA2 may be UspA2 from M. catarrhalis strain ATCC (a US registered trademark) 25238™, American 2933. American 2912, American 2908, Finnish 307, Finnish 353, Finnish 358, Finnish 216, Dutch H2, Dutch F10, Norwegian 1, Norwegian 13, Norwegian 20, Norwegian 25, Norwegian 27, Norwegian 36, BC5SV, Norwegian 14, Norwegian 3, Finish 414, Japanese Z7476, Belgium Z7530, German Z8063, American 012E, Greek MC317, American V1122, American P44, American V1171, American TTA24, American 035E, American SP12-6, American SP12-5, Swedish BC5, American 7169, Finnish FIN2344, American V1118, American V1145 or American V1156. UspA2 may be UspA2 as set forth in any of SEQ ID NO: 1—SEQ ID NO: 38. UspA2 may be UspA2 which has been isolated from human subjects which were isolated in the AERIS study (a clinical study wherein strains of M. catarrhalis were isolated from human subjects, see reference [Bourne S et al. Acute exacerbation and respiratory infectionS in COPD (AERIS): protocol for a prospective, obervational cohort study. BMJ open. 2014; 4(3):e004546].

UspA2 may be UspA2 from another source which corresponds to the sequence of UspA2 in any one of SEQ ID NO: 1—SEQ ID NO: 46 (or UsA2H and UspA2V sequence of SEQ ID NO: 49-52). Corresponding UspA2 sequences may be determined by one skilled in the art using various algorithms. For example, the Gap program or the Needle program may be used to determine UspA2 sequences corresponding to any one of SEQ ID NO: 1—SEQ ID NO: 38. UspA2 may be a sequence with at least 80% identity, over the entire length, to any of SEQ ID NO: 1—SEQ ID NO: 46.

Antibodies against UspA2 can be tested for functionality using the Serum Bactericidal Assay (for example as described in Example 5). Inclusion of a negative control strain (i.e. one which is UspA-null) confirms that any response observed is UspA-dependent. As an alternative the skilled person may test the functionality of an UspA2 fragment in vivo for example using techniques referred to in WO2015/125118A1. e.g. Mouse model of lung colonization (Example 8 of WO2015/125118A1.), Lung Challenge Model (Example 10 of WO2015/125118A1) or Immunogenicity in Mice (Example 11-13 of WO2015/125118A1.). Analysis of UspA2 specific Anti-IgG antibodies can be performed by ELISA as described in Example 11 (page 84) of WO2015/125118A1.

Reference to UspA1 herein may be UspA1 of SEQ ID NO: 47 (with signal peptide) or SEQ ID NO: 48 (without signal peptide) or a sequence with at least 60% similarity to SEQ ID NO: 47 or 48. Reference to UspA2H herein may be UspA2H of SEQ ID NO: 49 (with signal peptide) or SEQ ID NO: 50 (without signal peptide) or a sequence with at least 60% similarity to SEQ ID NO: 49 or 50. Reference to UspA2V herein may be UspA2V of SEQ ID NO: 51 (with signal peptide) or SEQ ID NO: 52 (without signal peptide) or a sequence with at least 60% similarity to SEQ ID NO: 51 or 52.

### Immunogenic Fragments and Constructs of UspA2

In an embodiment, UspA2 is an immunogenic fragment of the full-length UspA2 from M catarrhalis wherein the fragment comprises the amino acids that align with amino acids 30-540 of SEQ ID NO. 1 (SEQ ID NO: 53), amino acids 31-540 of SEQ ID NO: 1 (SEQ ID NO: 54), amino acids 30-519 of SEQ ID NO: 1 (SEQ ID NO: 55), amino acids 30-564 of SEQ ID NO: 1 (SEQ ID NO: 56) or amino acids 31-564 of SEQ ID NO: 1 (SEQ ID NO: 57). In an embodiment, UspA2 is an immunogenic fragment of UspA2 comprising at least 450 amino acids of the full length UspA2 (for example at least 450 amino acids of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 49, 50, 51 or 52.

In an embodiment, UspA2 may be an UspA2 protein construct which is produced with fragments of UspA2 with and without additional amino acids. Production of UspA2 protein constructs as described herein has been previously described, see for example WO2015/125118A1 the content of which is incorporated herein in its entirety. The following table describes protein constructs made.

TABLE 1
Protein constructs containing UspA2 protein.

| Construct ID | Description | N-terminal --------------------------------------------------C-Terminal | |
|---|---|---|---|
| MC-001 | UspA2 + 1/2 helix + 6His | UspA2 fragment | ASHHHHHH |
| | | (A.A .: 30-540 of SEQ ID NO: 1, SEQ ID NO: 53) | |
| | A.A. 30 540 541 548 | | |
| MC-002 | UspA2 + 1/2 helix | UspA2 fragment | |
| | | (A.A .: 30-540 of SEQ ID NO: 1, SEQ ID NO: 53) | |
| | A.A. 30 540 | | |
| MC-003 | UspA2 + 1/2 helix + 1His | UspA2 fragment | H |
| | | (A.A .: 30-540 of SEQ ID NO: 1, SEQ ID NO: 53) | |
| | A.A. 30 540 541 | | |
| MC-004 | UspA2 + 1/2 helix + 2His | UspA2 fragment | HH |
| | | (A.A .: 30-540 of SEQ ID NO:1, SEQ ID NO: 53) | |
| | A.A. 30 540 541 542 | | |
| MC-005 | UspA2 Δhelix + 6His | UspA2 fragment | ASHHHHHH |
| | | (A.A .: 30-519 of SEQ ID NO: 1, SEQ ID NO: 55) | |
| | A.A. 30 519 520 527 | | |
| MC-006 | UspA2 Δhelix | UspA2 fragment | |
| | | (A.A .: 30-519 of SEQ ID NO: 1, SEQ ID NO: 55) | |
| | A.A. 30 519 | | |
| MC-007 | UspA2 + helix + 6His | UspA2 fragment | ASHHHHHH |
| | | (A.A .: 30-564 of SEQ ID NO: 1, SEQ ID NO: 56) | |
| | A.A. 30 564 565 572 | | |
| MC-008 | UspA2 + helix + 2His | UspA2 fragment | HH |
| | | (A.A .: 30-564 of SEQ ID NO: 1, SEQ ID NO: 56) | |
| | A.A. 30 564 565 566 | | |
| MC-009 | UspA2 + helix + 2His ΔQ | UspA2 fragment | HH |
| | | (A.A .: 31-564 of SEQ ID NO: 1, SEQ ID NO: 57) | |
| | A.A. 31 564 565 566 | | |
| MC-010 | UspA2 + helix | UspA2 fragment | |
| | | (A.A .: 30-564 of SEQ ID NO: 1, SEQ ID NO: 56) | |
| | A.A. 30 564 | | |
| MC-011 | UspA2 + 1/2helix + 6HisΔQ | UspA2 fragment | ASHHHHHH |
| | | (A.A .: 31-540 of SEQ ID NO: 1, SEQ ID NO: 54) | |

A.A. 31 540 541 548

A.A. = amino acid

In an embodiment the protein constructs MC-001, MC-002, MC-003, MC-004, MC-005, MC-006, MC-007, MC-008, MC-009, MC-010 and MC-011 in table 1 above, further comprise a methionine at the amino terminal. The DNA and amino acid sequences (SEQ ID NOs) for each of the protein constructs listed in Table 1 are set out in Table 2 below.

TABLE 2
Immunogenic Fragments/Constructs of UspA2 from Moraxella catarrhalis.
(M) = Methionine, (ASHHHHHH, SEQ ID NO: 97) = Ala, Ser, His, His, His,
His, His, His, (H) = His, (HH) = His His

| | DNA SEQ ID | AMINO ACID | Construct (UspA2 numbering |
|---|---|---|---|
| Fragment | NO: | SEQ ID NO: | according to SEQ ID NO: 1). |
| MC-001 | SEQ ID NO: 58 | SEQ ID NO: 59 | (M)(UspA2 amino acids 30-540) |
| | | | (ASHHHHHH) |
| MC-002 | SEQ ID NO: 60 | SEQ ID NO: 61 | (M)(UspA2 amino acids 30-540) |
| MC-003 | SEQ ID NO: 62 | SEQ ID NO: 63 | (M)(UspA2 amino acids 30-540)(H) |
| MC-004 | SEQ ID NO: 64 | SEQ ID NO: 65 | (M)(UspA2 amino acids 30-540)(HH) |
| MC-005 | SEQ ID NO: 66 | SEQ ID NO: 67 | (M)(UspA2 amino acids 30-519) |
| | | | (ASHHHHHH) |
| MC-006 | SEQ ID NO: 68 | SEQ ID NO: 69 | (M)(UspA2 amino acids 30-519) |
| MC-007 | SEQ ID NO: 70 | SEQ ID NO: 71 | (M)(UspA2 amino acids 30-564) |
| | | | (ASHHHHHH) |
| MC-008 | SEQ ID NO: 72 | SEQ ID NO: 73 | (M)(UspA2 30-564)(HH) |
| MC-009 | SEQ ID NO: 74 | SEQ ID NO: 75 | (M)(UspA2 31-564)(HH) |
| MC-010 | SEQ ID NO: 76 | SEQ ID NO: 77 | (M)(UspA2 amino acids 30-564) |
| MC-011 | SEQ ID NO: 78 | SEQ ID NO: 79 | (M)(UspA2 amino acids 31-540) |
| | | | (ASHHHHHH) |

In an embodiment, UspA2 is a protein construct having at least 70%, 80%, 85%, 90%, 91%, 92%, 93%, 94%, 95%, 96%, 97%, 98%, 99%, or 100% to MC-009 SEQ ID NO. 75 (corresponding to SEQ ID NO: 69 of WO2015/125118A1). In another embodiment, UspA2 is a protein construct having an amino acid sequence of SEQ ID NO. 75 (corresponding to SEQ ID NO: 69 of WO2015/125118A1).

### Antibodies

The present invention provides antigen binding proteins (for example antibodies) which bind UspA2. Unless otherwise stated, amino acid numbering in relation to UspA2 is in respect of UspA2 of SEQ ID NO: 1.

UspA2 Monoclonal Antibody (mAb)

The present invention provides an antibody which binds to UspA2 at an epitope within the consensus sequence of YNELQD-[A/Q]-YA-[QK/KQ]-QTE (SEQ ID NO: 82), e.g. SEQ ID NO: 83, 84, 85 or 86. In an embodiment the antibody is a mouse monoclonal antibody (mAB). In an embodiment the isotype of the mAb is a mouse IgG2A. In an embodiment, the mAb is FHUSPA2/10. In an embodiment the antibody of the invention is produced by the Repetitive Immunisation Multiple Sites (RIMMS) method which is described in (Eric P. Dixon. Cell Biology (Third Edition) A Laboratory Handbook: Chapter 58—Rapid Development of Monoclonal Antibodies Using Repetitive Immunizations, Multiple Sites. Academic Press. 2006; 1:483-90) and is incorporated herein by reference.

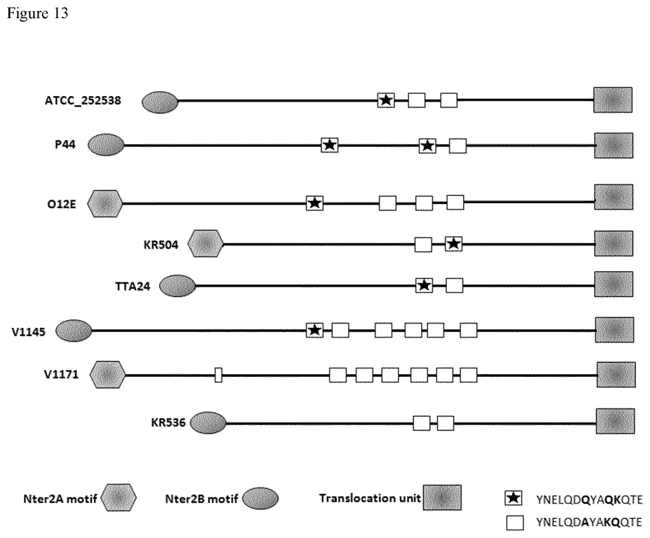

The antibody binds to UspA2 at an epitope within the consensus sequence of SEQ ID NO: 82 (e.g. SEQ ID NO: 83, 84, 85 or 86). As the epitope of the invention is within the consensus sequence of the invention the antibody also binds the epitope. The consensus sequence of the invention (i.e. SEQ ID NO: 82) may be present at multiple locations along the length of the UspA2 amino acid sequence (e.g. of SEQ ID NO: 1). The antibody is thus able to bind to the consensus sequence and/or epitope of the invention at multiple sites (for example, up to 15 locations within the Stalk region of UspA2). The antibody of the invention is also able to bind UspA1. The antibody can promote UspA2 intermolecular bridging and can bind secondary motifs and repeat regions. The location of the consensus sequence of the invention in various circulating strains of M. catarrhalis is shown in FIG. 13.

In an embodiment, the antibody which binds to UspA2 at an epitope within the consensus sequence of SEQ ID NO: 82 (e.g. SEQ ID NO: 83, 84, 85 or 86) comprises (i) any one or a combination of Complementarity-determining regions (CDRs) selected from CDRH1, CDRH2, CDRH3 from SEQ ID NO: 94, and/or CDRL1, CDRL2, CDRL3 from SEQ ID NO: 96; or (ii) a CDR variant of (i), wherein the variant has 1, 2, or 3 amino acid modifications in each CDR.

In an embodiment, the antibody which binds to UspA2 at an epitope within the consensus sequence of SEQ ID NO: 82 (e.g. SEQ ID NO: 83, 84, 85 or 86) comprises any one or a combination or all of the following CDRs: (a) CDRH1 of SEQ ID NO: 87; (b) CDRH2 of SEQ ID NO: 88; (c) CDRH3 of SEQ ID NO: 89; (d) CDRL1 of SEQ ID NO: 90; (e) CDRL2 of SEQ ID NO: 91; and/or (f) CDRL3 of SEQ ID NO: 92. The CDRs were determined by Kabat. The antibody may comprise: a humanised VH region, or a humanised Heavy Chain (HC) sequence; and/or a humanised VL region, or a humanised Light Chain (LC) sequence, which comprise the CDRs as described above.

In an embodiment, the antibody which binds to UspA2 at an epitope within the consensus sequence of SEQ ID NO: 82 (e.g. SEQ ID NO: 83, 84, 85 or 86) comprises a Variable Heavy (VH) region comprising a sequence at least 80% identical (e.g. at least 85%, 90%, 91%, 92%, 93%, 94%, 95%, 96%, 97%, 98%, 99% or 100% identical) to SEQ ID NO: 94; and a Variable Light (VL) region comprising a sequence at least 80% identical (e.g. at least 85%, 90%, 91%, 92%, 93%, 94%, 95%, 96%, 97%, 98%, 99% or 100% identical) to SEQ ID NO: 96. In an embodiment the antibody which binds to UspA2 at an epitope within the consensus sequence of SEQ ID NO: 82 (e.g. SEQ ID NO: 83, 84, 85 or 86) comprises a Variable Heavy (VH) region encoded by sequence at least 80% identical (e.g. at least 85%, 90%, 91%, 92%, 93%, 94%, 95%, 96%, 97%, 98%, 99% or 100% identical) the sequence of SEQ ID NO: 93; and/or a Variable Light (VL) region encoded by a sequence at least 80% identical (identical (e.g. at least 85%, 90%, 91%, 92%, 93%, 94%, 95%, 96%, 97%, 98%, 99% or 100% identical) to the sequence of SEQ ID NO: 95.

The antibody sequence may be a variant sequence with up to 3 amino acid modifications. For example, the modification is a substitution, addition or deletion. For example, the variant sequence may have up to 3, 2 or 1 amino acid substitution(s), addition(s) and/or deletion(s). The sequence variation may exclude the CDR(s), for example the CDR(s) is intact, and the variation is in the remaining portion of the VH or VL (or HC or LC) sequence, so that the CDR sequence is fixed. The variant sequence substantially retains the biological characteristics of the unmodified antibody.

As used herein the term “VH Region” or “VL Region” refers to the variable portions of the heavy (VH) and light (VL) chains respectively. These regions form the binding pocket, which binds the specific antigens, and contains the major diversity of the immunoglobulin.

In an embodiment the antibody which binds to UspA2 at an epitope within the consensus sequence of SEQ ID NO: 82 (e.g. SEQ ID NO: 83, 84, 85 or 86) comprises a VH region of SEQ ID NO: 94 and a VL region of SEQ ID NO: 96. In an embodiment the antibody which binds to UspA2 at an epitope within the consensus sequence of SEQ ID NO: 82 (e.g. SEQ ID NO: 83, 84, 85 or 86) comprises a VH region encoded by the sequence of SEQ ID NO: 93; and/or a VL region encoded by the sequence of SEQ ID NO: 95.

In an embodiment the antibody which binds to UspA2 at an epitope within the consensus sequence of SEQ ID NO: 82 (e.g. SEQ ID NO: 83, 84, 85 or 86) is able to promote bactericidal activity.

The invention further provides an antibody that binds to UspA2, and competes for binding to the consensus sequence SEQ ID NO: 82 with a reference antibody with a VH region comprising SEQ ID NO: 94 and a VL region comprising SEQ ID NO: 96. Suitable assays to analyse whether antibodies compete for binding are well known in the art (for example see Kwak & Yoon et al 1996, J Immunol Methods 191(1): 49-54).

The binding of the antibody of the invention to an epitope within the consensus sequence of SEQ ID NO: 82, can be determined using Hydrogen-Deuterium exchange coupled with Mass Spectrometry (HDX-MS). Briefly, HDX-MS detects structural changes of a protein due to ligand binding, protein-protein interaction, post-translational modifications and others (the method is described in Example 3). The epitope region on the UspA2 which is targeted by antibody will display reduced exchange rates in the presence of the antibody relative to UspA2 alone which can be identified by HDX-MS. Following the exchange, the reaction is quenched with an acidic pH and low temperature. The proteins are digested with pepsin or other acidic proteases and analysed via mass spectrometry.

The present invention also provides an expression vector comprising the nucleic acid sequence (encoding the antibody of the invention) as defined herein. The present invention also provides a recombinant host cell comprising the nucleic acid sequence as defined herein, or the expression vector as defined herein. The present invention also provides a method for the production of the antigen binding protein as defined herein, which method comprises culturing the host cell as defined herein under conditions suitable for expression of said nucleic acid sequence or vector, whereby the antigen binding protein is expressed and purified. The present invention also provides an antigen binding protein produced by the method described herein. The present invention also provides a pharmaceutical composition comprising the antigen binding protein as defined herein, and one or a combination of pharmaceutically acceptable carriers, excipients or diluents.

In a further embodiment the antibody which binds to UspA2 at an epitope within the consensus sequence of SEQ ID NO: 82 (e.g. SEQ ID NO: 83, 84, 85 or 86) is capable of binding to UspA2 when UspA2 is present as a fragment or protein construct. For example, the antibody may bind to any of the UspA2 protein constructs as described in SEQ ID NO: 59 (MC-001), 61 (MC-002), 63 (MC-003), 65 (MC-004), 67 (MC-005), 69 (MC-006), 71 (MC-007), 73 (MC-008), 75 (MC-009), 77 (MC-010) or 79 (MC-011) or a sequence with at least 80% similarity to any of SEQ ID NO: 59 (MC-001), 61 (MC-002), 63 (MC-003), 65 (MC-004), 67 (MC-005), 69 (MC-006), 71 (MC-007), 73 (MC-008), 75 (MC-009), 77 (MC-010) or 79 (MC-011).

In an embodiment the antibody which binds to UspA2 at an epitope within the consensus sequence of SEQ ID NO: 82 (e.g. SEQ ID NO: 83, 84, 85 or 86) binds to the UspA2 protein construct of SEQ ID NO: 75 (MC-009) or sequences with at least 80% identity to SEQ ID NO: 75.

A person skilled in the art will understand that when the UspA2 amino acid sequence is a variant and/or fragment of an amino acid sequence of SEQ ID NO. 1, such as an amino acid sequence at least 80%, 85%, 90%, 92%, 95%, 96%, 97%, 98% or 99% identical to SEQ ID NO. 1, reference to the location of the consensus sequence of SEQ ID NO: 82 may vary. Variants of SEQ ID NO.1 could lead to a difference in the actual amino acid position of the consensus sequence in the sequence, however, by lining the sequence up with the reference sequence, the amino acid in in an equivalent position to the corresponding amino acid in the reference sequence can be identified and hence the appropriate amino acids identified.

In an embodiment the antibody which binds to UspA2 at an epitope within the consensus sequence of SEQ ID NO: 82 (e.g. SEQ ID NO: 83, 84, 85 or 86) binds to an epitope that is associated with an immunogenically active form of UspA2. In an embodiment the antibody binds to an epitope that is associated with UspA2 in a conformation where it is immunogenically active. In an embodiment the antibody binds to an epitope that is associated with an immunogenically active UspA2 protein construct (e.g. MC-009 of SEQ ID NO: 75). In an embodiment the antibody binds to an epitope within or comprising amino acid residues Y279 to E292, Y314 to E327 and Y349 to E362 of the UspA2 protein construct MC-009 (of SEQ ID NO: 75) wherein said protein construct is for use in a vaccine. In an embodiment the antibody which binds to UspA2 at an epitope within the consensus sequence of SEQ ID NO: 82 (e.g. SEQ ID NO: 83, 84, 85 or 86) binds (or preferentially binds) to an UspA2 antigen which is capable of eliciting an immune response in a mammal, preferably in a human being. In an embodiment, the antibody binds (or preferentially binds) to an UspA2 antigen which is protective against diseases associated with M catarrhalis. In an embodiment, the antibody binds (or preferentially binds) to an UspA2 antigen which is protective against AECOPD.

In an embodiment the antibody which binds to UspA2 at an epitope within the consensus sequence of SEQ ID NO: 82 binds to an epitope that is associated with the native conformation of UspA2. In an embodiment the antibody binds to UspA2 in its native conformation with a higher specificity and/or affinity than to UspA2 in a non-native conformation. In an embodiment, the antibody binds to UspA2 in its native conformation with higher affinity than to UspA2 in a non-native conformation. In an embodiment, the antibody binds to UspA2 in its native conformation with higher specificity than to UspA2 in a non-native conformation

In an embodiment the antibody which binds to UspA2 at an epitope within the consensus sequence of SEQ ID NO: 82, (e.g. SEQ ID NO: 83, 84, 85 or 86) is unable to bind to UspA2 in its non-native (or significantly non-native) conformation or less antibody is capable of binding UspA2 in its non-native conformation. In an embodiment the antibody binds to UspA2 in its non-native (or significantly non-native) conformation with less specificity and/or affinity than to UspA2 in its native conformation. For example, the antibody which binds to UspA2 at an epitope within the consensus sequence of SEQ ID NO: 82 binds to UspA2 in its native (or substantially native) conformation with higher specificity and/or higher affinity than to a UspA2 which is denatured.

In an embodiment an UspA2 antigen or test sample comprising UspA2 may be denatured or adopt a non-native conformation for example via thermal stress, freeze-thawing, pH alterations, oxidation, enzymatic digestion, mishandling or process errors. In an embodiment, an UspA2 antigen or test sample comprising UspA2 may be denatured or adopt a non-native conformation for example via reduction (e.g. methionine reduction) or via light exposure In an embodiment UspA2 may be denatured via thermal stress. In an embodiment, UspA2 may be denatured following exposure to temperatures greater than room temperature (i.e. greater than approximately 20° C. to 22° C.), greater than 30° C., greater than 40° C., greater than 50° C., greater than 60° C. or greater than 70° C. In an embodiment, UspA2 may be denatured at 45° C.±5° C. In an embodiment, UspA2 may be denatured via thermal stress for up to 1 hour, up to 2 hours, up to 4 hours, up to 6 hours, up to 8 hours, up to 12 hours, up to 24 hours. In an embodiment, UspA2 may be denatured via thermal stress for greater than 24 hours.

In an embodiment the antibody which binds to UspA2 at an epitope within the consensus sequence of SEQ ID NO: 82 binds to a vaccine sample comprising an UspA2 antigen in its native conformation with a higher specificity and/or affinity as compared to a vaccine sample comprising a UspA2 antigen which has lost the relevant epitope or where the relevant epitope has been modified. In an embodiment, the UspA2 antigen has lost the relevant epitope (or the epitope has been modified) for example due to denaturation, aggregation or breakdown during storage or by mishandling.

### UspA2 Polyclonal Antibodies

The present invention provides a polyclonal antibody (pAb) which binds to UspA2 at an epitope within the region of SEQ ID NO: 81 (i.e. A500-L551 of SEQ ID NO: 1). In an embodiment the UspA2 polyclonal antibody of the invention binds to UspA2 at an epitope within the sequence as set forth in SEQ ID NO: 81.

In an embodiment the pAb which binds to UspA2 is a produced in one of either chicken, goat, guinea pig, hamster, horse, mouse, rat or sheep. In an embodiment the pAb is produced in rabbit. In an embodiment the pAb is an anti-UspA2 rabbit pAb. In an embodiment the pAb is produced from antisera of rabbits immunized with an UspA2 antigen. In an embodiment the pAb is produced from antisera of rabbits immunized with MC-009 (of SEQ ID NO: 75).

In an embodiment the pAb which binds to UspA2 binds to an epitope comprising or consisting of i) SEQ ID NO: 81 or ii) variants of SEQ ID NO: 81, wherein said variants comprise 1, 2, 3, 4 or 5 amino acid modifications. Said amino acid modifications are single amino acid modifications, i.e. 1 single amino acid modification, 2 single amino acid modifications, 3 single amino acid modifications etc.

In an embodiment, the pAb which binds to UspA2 binds to an epitope within the sequence comprising or consisting of amino acid residues A500 to L551 (e.g. SEQ ID NO: 81) of UspA2. In an embodiment, the pAb binds to an epitope consisting of amino acid residues A500 to L551 (e.g. SEQ ID NO: 81) of UspA2. Reference to amino acid residues A500 to L551 of UspA2 relate to the UspA2 sequence as defined in SEQ ID NO:1.

In an embodiment, the pAb which binds to UspA2 substantially binds to an immune dominant epitope located within SEQ ID NO: 81 (A500 to L551 of SEQ ID NO: 1). In an embodiment the core of the immune dominant epitope is located at D538-F544 of SEQ ID NO: 1. In an embodiment the core of the immune dominant epitope is located at D509-F515 of the UspA2 protein construct as set forth in SEQ ID NO: 75 (MC-009).

Amino acid residue ranges referred to herein (e.g. A500 to L551 of SEQ ID NO: 1) includes the “end” amino acid residues A500 and L551 as well as any (or all) residues within said ranges. In an embodiment the UspA2 polyclonal antibody of the invention may bind to any residues within region A500 to L551 of UspA2.

In a further embodiment the pAb which binds to UspA2 is capable of binding to UspA2 when UspA2 is present as a fragment or protein construct. For example, the pAb may bind to any of the UspA2 protein constructs of SEQ ID NO: 59 (MC-001), 61 (MC-002), 63 (MC-003), 65 (MC-004), 67 (MC-005), 69 (MC-006), 71 (MC-007), 73 (MC-008), 75 (MC-009), 77 (MC-010) or 79 (MC-011) or a sequence with at least 80% similarity to any of SEQ ID NO: 59 (MC-001), 61 (MC-002), 63 (MC-003), 65 (MC-004), 67 (MC-005), 69 (MC-006), 71 (MC-007), 73 (MC-008), 75 (MC-009), 77 (MC-010) or 79 (MC-011). In an embodiment pAb binds to SEQ ID NO: 75 (MC-009) or sequences with at least 80% identity to SEQ ID NO: 75. In an embodiment the pAb binds to UspA2 at one or more of amino acid residues within the region A471-L522 of the UspA2 protein construct of SEQ ID NO: 75 (MC-009) or a sequence with at least 70%, at least 75%, at least 80%, at least 85%, at least 90%, at least 95% or at least 100% identity to SEQ ID NO: 75.

It will be understood by a person skilled in the art, that reference to amino acid residues within A500 to L551 of UspA2 is referring to the full length UspA2 as defined in SEQ ID NO:1. Furthermore, reference to amino acid residues within A500 to L551 of UspA2 is referring to the amino acid number counting consecutively from the N-terminus of the amino acid sequence, of SEQ ID NO. 1. Amino acid residues within A500 to L551 refers to the amino acids from the₅₀₀th to 551^{st} amino acid of SEQ ID NO. 1.

A person skilled in the art will understand that when the UspA2 amino acid sequence is a variant and/or fragment of an amino acid sequence of SEQ ID NO. 1, such as an amino acid sequence at least 80%, 85%, 90%, 92%, 95%, 96%, 97%, 98% or 99% identical to SEQ ID NO. 1, the reference to A500 to L551 refers to a the position that would be equivalent to the defined position, if this sequence was lined up with an amino acid sequence of SEQ ID NO. 1 in order to maximise the sequence identity between the two sequences (Sequence alignment tools are not limited to Clustal Omega (www(.)ebi(.)ac(.)ac(.)uk) MUSCLE (www(.)ebi(.)ac(.)uk), or T-coffee (www(.)tcoffee(.)org). In one aspect, the sequence alignment tool used is Clustal Omega (www(.)ebi(.)ac(.)ac(.)uk). Variants of SEQ ID NO.1 could lead to a difference in the actual amino acid position of the consensus sequence in the sequence, however, by lining the sequence up with the reference sequence, the amino acid in in an equivalent position to the corresponding amino acid in the reference sequence can be identified and hence the appropriate amino acids identified.

Reference to amino acid residues within A500-L551 may also be referring to the corresponding residues within a fragment of UspA2 (such as the fragments described in SEQ ID NO: 53—SEQ ID NO: 57). This is only to the extent that the corresponding amino acids A500-L551 remain present in the sequence i.e. wherein said fragment retains the region outlined in SEQ ID NO: 81 or a substantial enough portion of the region outline in SEQ ID NO: 81 such that the antibody still binds.

In an embodiment the pAb which binds to UspA2 binds to an epitope that is associated with an immunogenically active form of UspA2. In an embodiment the pAb binds to an epitope that is associated with UspA2 in a conformation where it is immunogenically active. In an embodiment the pAb binds to an epitope that is associated with an immunogenically active UspA2 protein construct (e.g. MC-009 of SEQ ID NO: 75). In an embodiment the pAb binds to an epitope within or comprising amino acid residues A471-L522 of the immunogenically active UspA2 protein construct MC-009 (of SEQ ID NO: 75) wherein said protein construct is for use in a vaccine. In an embodiment the pAb binds (or preferentially binds) to an UspA2 antigen (e.g. MC-009 of SEQ ID NO: 75) which is capable of eliciting an immune response in a mammal, preferably in a human being. In an embodiment, the pAb binds (or preferentially binds) to a UspA2 antigen which is protective against diseases associated with M. catarrhalis. In an embodiment, the pAb binds (or preferentially binds) to an UspA2 antigen which is protective against AECOPD.

In an embodiment the pAb which binds to UspA2 binds to an epitope that is associated with the native conformation of UspA2. In an embodiment the pAb binds to UspA2 in its native conformation with a higher specificity and/or affinity than to UspA2 in a non-native conformation. In an embodiment, the pAb binds to UspA2 in its native conformation with higher affinity than to UspA2 in a non-native conformation. In an embodiment, the pAb binds to UspA2 in its native conformation with higher specificity than to UspA2 in a non-native conformation.

In an embodiment the pAb which binds to UspA2 is unable to bind to UspA2 in its non-native (or significantly non-native) conformation or less UspA2 polyclonal antibody of the invention is capable of binding UspA2 in its non-native conformation. In an embodiment the pAb binds to UspA2 in its non-native (or significantly non-native) conformation with less specificity and/or affinity than to UspA2 in its native conformation. For example, the pAb binds to UspA2 in its native (or substantially native) conformation with higher specificity and/or higher affinity than to a UspA2 which is denatured.

In an embodiment an UspA2 antigen or test sample comprising UspA2 may be denatured or adopt a non-native conformation for example via thermal stress, freeze-thawing, pH alterations, oxidation, enzymatic digestion, mishandling or process errors. In an embodiment UspA2 may be denatured via thermal stress. In an embodiment, UspA2 may be denatured following exposure to temperatures greater than room temperature (i.e. greater than approximately 20° C. to 22° C.), greater than 30° C., greater than 40° C., greater than 50° C., greater than 60° C. or greater than 70° C. In an embodiment, UspA2 may be denatured at 65° C.+5° C. In an embodiment, UspA2 may be denatured via thermal stress for up to 1 hour, up to 2 hours, up to 4 hours, up to 6 hours, up to 8 hours, up to 12 hours, up to 24 hours. In an embodiment, UspA2 may be denatured via thermal stress for greater than 24 hours.

In an embodiment the pAb which binds to UspA2 binds to a vaccine sample comprising an UspA2 antigen in its native conformation with a higher specificity and/or affinity as compared to a vaccine sample comprising a UspA2 antigen which has lost the relevant epitope. In an embodiment, the UspA2 antigen has lost the relevant epitope for example due to denaturation, aggregation or breakdown during storage or by mishandling. In an embodiment, the UspA2 antigen has lost the epitope within the region outlined in SEQ ID NO: 81 due to denaturation, aggregation or breakdown during storage or mishandling.

In an embodiment the pAb which binds to UspA2 is cross-bactericidal against heterologous strains of M. catarrhalis.

In a further aspect, there is provided an antigen binding protein (e.g. an antibody) that binds to UspA2 and competes for binding at one or more of amino acid residues within A500 to L551 of SEQ ID NO: 1 i.e. competes for binding to an epitope within the sequence as set forth in SEQ ID NO: 81. Suitable assays to analyse whether antibodies compete for binding are well known in the art (for example see Kwak & Yoon et al 1996, J Immunol Methods 191(1): 49-54).

The binding of the pAb at one or more of amino acid residues within A500 to L551, can be determined using Hydrogen-Deuterium exchange coupled with Mass Spectrometry (HDX-MS). Briefly, HDX-MS detects structural changes of a protein due to ligand binding, protein-protein interaction, post-translational modifications and others (the method is described in Example 2 and 4). The epitope region on the UspA2 sample which is targeted by the rabbit UspA2 polyclonal antibody will display reduced exchange rates in the presence of the anti-UspA2 rabbit pAb relative to UspA2 alone which can be identified by HDX-MS. Following the exchange, the reaction is quenched with an acidic pH and low temperature. The proteins are digested with pepsin or other acidic proteases and analysed via mass spectrometry.

The present invention also provides a method for the production of the anti-UspA2 rabbit pAb. Said method includes the immunization of rabbits on up to five occasions (for example, one immunization, two immunizations, thee immunizations etc.) with UspA2 (adjuvanted or unadjuvanted) and bleeding after a defined period of time (for example 42 days post immunization and 49 days post immunization). The present invention also provides an antigen binding protein produced by the method described herein. The present invention also provides a pharmaceutical composition comprising the antigen binding protein as defined herein, and one or a combination of pharmaceutically acceptable carriers, excipients or diluents.

### Use of Antibodies in an Assay

The role of a potency assay (for example an in vitro relative potency assay or IVRP assay) is to ensure that an antigen contains the appropriate biochemical properties to elicit the needed immune response. The present invention provides a new enzyme-linked immunosorbent assay (ELISA) method to appraise the suitability of UspA2 antigens for use in a vaccine. A number of quality control assessments need to be conducted for each batch of vaccine to certify that they safe and effective for the public. Potency assays are a crucial vaccine release assay.

The present invention relates to the use of antibodies that are capable of binding to UspA2 in an assay for the detection and measurement of UspA2 in a test sample. In particular the invention relates to the detection or measurement of a change in the conformation of UspA2 in a test sample which may indicate whether said UspA2 is suitable for release to the public as a vaccine component.

Because detection of, or measurement of a change in the conformation of UspA2 relies upon the UspA2 antibodies described herein, said use is considered to be a good predictor of in vivo potency since said antibodies bind biologically relevant, functional epitopes. In an embodiment, determining or measuring the presence of UspA2 in its native conformation involves determining or measuring the presence of UspA2 in a form which is suitable for administration to a patient (e.g. as a component of an immunogenic composition).

The invention therefore provides the use of an antibody which binds to UspA2 in an assay for the detection of, or measurement of a change in, the conformation of UspA2 wherein UspA2 is present in a test sample. The invention further provides the use of an antibody which binds to UspA2 to determine or measure the potency of a test sample comprising UspA2. Thus, in an embodiment there is provided the use of an antibody which binds to UspA2 in an assay for the detection of, or measurement of the potency of UspA2 wherein UspA2 is present in a test sample.

In an embodiment, the antibody which binds to UspA2 is a monoclonal antibody, optionally an IgG2A mouse monoclonal antibody. In an embodiment the antibody which binds to UspA2 comprises a VH region comprising a sequence at least 80% identical to SEQ ID NO: 94 and a VL region comprising a sequence at least 80% identical to SEQ ID NO: 96. In an embodiment, the antibody which binds to UspA2 comprises a VH region of SEQ ID NO: 94 and a VL region of SEQ ID NO: 96.

In an embodiment, the antibody which binds to UspA2 binds an epitope within the consensus sequence of SEQ ID NO: 82 (e.g. SEQ ID NO: 83, 84, 85 or 86)

In an embodiment, the antibody which binds to UspA2 comprises any one or a combination of CDRs selected from (i) CDRH1 (SEQ ID NO: 87), CDRH2 (SEQ ID NO: 88), CDRH3 (SEQ ID NO: 89), CDRL1 (SEQ ID NO: 90), CDRL2 (SEQ ID NO: 91) and CDRH3 (SEQ ID NO: 92) or (ii) a CDR variant of (i), wherein the variant has 1, 2, or 3 amino acid modifications in each CDR, which is able to bind to an epitope within the consensus sequence of SEQ ID NO: 82.

In an embodiment the UspA2 which is present in a test sample comprises a sequence with at least 80% identity (e.g. at least 85% identity, at least 90% identity, at least 95% identity) to any one of SEQ ID NO: 59 (MC-001), 61 (MC-002), 63 (MC-003), 65 (MC-004), 67 (MC-005), 69 (MC-006), 71 (MC-007), 73 (MC-008), 75 (MC-009), 77 (MC-010) or 79 (MC-011). In an embodiment the antibody which binds to UspA2 detects or measures a change in the conformation of UspA2 of SEQ ID NO: 59 (MC-001), 61 (MC-002), 63 (MC-003), 65 (MC-004), 67 (MC-005), 69 (MC-006), 71 (MC-007), 73 (MC-008), 75 (MC-009), 77 (MC-010) or 79 (MC-011). In an embodiment, the UspA2 which is present in a test sample is UspA2 of SEQ ID NO: 75 (MC-009)

In an embodiment, at least one antibody (optionally, 1, 2, 3 or more antibodies) is/are used in the detection of or measurement of a change in the conformation of UspA2. In an embodiment, at least one antibody (optionally, 1, 2, 3 or more antibodies) is/are used to determine or measure the potency of a test sample comprising UspA2.

In an embodiment, two antibodies are used in the detection or measurement of a change in the conformation of UspA2. In an embodiment, two antibodies are used to determine or measure the potency of a test sample comprising UspA2. In an embodiment the two antibodies bind to non-overlapping epitopes of UspA2.

### Assay

The role of a potency assay is to ensure that an antigen contains the appropriate biochemical properties to elicit the needed immune response. The in vitro relative potency assay described herein may be used for drug-product release and stability testing of an NTHi-Mcat vaccine.

In a further aspect of the invention there is provided an assay comprising the steps of contacting a test sample comprising UspA2 with a first antibody and a second antibody to form a first antibody-UspA2-second antibody complex; and detecting or measuring the amount of said first antibody-UspA2-second antibody complex. In an embodiment, there is provided an assay comprising the steps of contacting a test sample comprising UspA2 with a first antibody and a second antibody to form a first antibody-UspA2-second antibody complex; and detecting or measuring the amount of said UspA2 in said test sample.

The invention provides a binding immunoassay. The invention can use any ELISA format, including those conventionally known as direct ELISA, indirect ELISA, sandwich ELISA, and competitive ELISA. In an embodiment the assay is a sandwich ELISA.

Step one of the ELISA assay of the invention involves permitting an UspA2 antigen within a test sample to interact with an antibody (optionally a monoclonal antibody). The interaction between the antibody and the immunogen is then detected. The interaction can be measured quantitatively, such that the ELISA provides a result which indicates the concentration of the antibody's target epitope within the vaccine sample. By using a monoclonal antibody which binds to a bactericidal epitope, the result indicates the concentration of the corresponding functional epitope in the vaccine sample and can distinguish between immunogens which retain the relevant epitope (and function) and those which have lost the epitope (e.g. due to denaturation, aggregation or breakdown during storage or by mishandling). By comparison with values obtained with a standard vaccine of known potency, results can be used to calculate relative potency of a test sample.

In an embodiment one of said first antibody or said second antibody is immobilized on a solid support. In an embodiment said first antibody or said second antibody that is immobilized on a solid support is also referred to as the capture antibody. In an embodiment, the first antibody is immobilized on a solid support and binds to a functional epitope on UspA2 when a test sample comprising UspA2 is added. In an embodiment, one of said first antibody or said second antibody is immobilized (i.e. is coated) on a solid support wherein said solid support is the bottom of the well of a microtiter plate. Said first antibody or said second antibody may be immobilized on a solid support (or surface) which may take various forms. Usually said antibody is immobilized on a plastic surface, such as a surface made from polystyrene, polypropylene, polycarbonate, or cyclo-olefin. The plastic will usually be transparent and colorless, particularly when using chromogenic enzyme substrates. White or black plastics may be preferred used when using luminescent or fluorescent substrates, as known in the art. The plastic will generally be used in the form of a microwell plate (microtiter plate) as known in the art for ELISA (a flat plate having multiple individual and reaction wells). Such plates include those with 6, 24, 96, 384 or 1536 sample wells, usually arranged in a 2:3 rectangular matrix.

In an embodiment, one of said first antibody or said second antibody is immobilized on a solid support wherein said immobilization is conducted for between 15 minutes and 120 minutes at 2-8° C., room temperature or 37° C. In an embodiment, one of said first antibody or said second antibody is immobilized on a solid support wherein said first antibody or said second antibody is diluted prior to immobilization. In an embodiment said first antibody or said second antibody is diluted in water, phosphate buffered saline (PBS), tris buffered saline (TBS) or blocking buffer (e.g. non-fat dried milk, BSA etc.). In an embodiment, one of said first antibody or said second antibody is diluted to a concentration of between 0.01-0.1 gg/ml, 0.01-0.5 gg/ml, 0.1-1 gg/ml, 0.5-2 gg/ml, 0.5-5 gg/ml, 1-3 gg/ml or 1-10 gg/ml.

In an embodiment said first antibody and said second antibody bind non-overlapping epitopes on UspA2. In an embodiment said first antibody and said second antibody do not compete for binding to UspA2. In an embodiment, binding of said first antibody to UspA2 does not adversely impact binding of said second antibody to UspA2. In an embodiment, binding of said second antibody to UspA2 does not adversely impact the bound first antibody. In an embodiment one of said first or said second antibody binds to UspA2 in the stalk domain. In an embodiment, one of said first or said second antibody binds to UspA2 to the C-terminal end of the stalk region immediately preceding the translocation domain of the outer membrane p-barrel.

In an embodiment one of said first antibody or said second antibody is a monoclonal antibody. In an embodiment one of said first antibody or said second antibody comprises a VH region comprising a sequence at least 80% identical to SEQ ID NO: 94 and a VL region comprising a sequence at least 80% identical to SEQ ID NO: 96. In an embodiment one of said first antibody or said second antibody comprises a VH region of SEQ ID NO: 94 and a VL region of SEQ ID NO: 96. In an embodiment, said first antibody comprises a VH region of SEQ ID NO: 94 and a VL region of SEQ ID NO: 96

In an embodiment one of said first antibody or said second antibody binds to UspA2 at an epitope within a consensus sequence of YNELQD-[A/Q]-YA-[QK/KQ]-QTE (SEQ ID NO: 82), e.g. SEQ ID NO: 83, 84, 85 or 86. In an embodiment said antibody binds to UspA2 wherein UspA2 is a protein construct of SEQ ID NO: 75 (MC-009). In an embodiment said first antibody binds to UspA2 at an epitope within a consensus sequence of YNELQD-[A/Q]-YA-[QK/KQ]-QTE (SEQ ID NO: 82), e.g. SEQ ID NO: 83, 84, 85 or 86.

In an embodiment one of said first antibody or said second antibody comprises any one or a combination of CDRs selected from (i) CDRH1 (SEQ ID NO: 87), CDRH2 (SEQ ID NO: 88), CDRH3 (SEQ ID NO: 89), CDRL1 (SEQ ID NO: 90), CDRL2 (SEQ ID NO: 91) and CDRL3 (SEQ ID NO: 92) or (ii) a CDR variant of (i), wherein the variant has 1, 2, or 3 amino acid modifications in each CDR, which is able to bind to an epitope within the consensus sequence of SEQ ID NO: 82. In an embodiment said first antibody comprises any one or a combination of CDRs selected from (i) CDRH1 (SEQ ID NO: 87), CDRH2 (SEQ ID NO: 88), CDRH3 (SEQ ID NO: 89), CDRL1 (SEQ ID NO: 90), CDRL2 (SEQ ID NO: 91) and CDRL3 (SEQ ID NO: 92) or (ii) a CDR variant of (i), wherein the variant has 1, 2, or 3 amino acid modifications in each CDR, which is able to bind to an epitope within the consensus sequence of SEQ ID NO: 82.

In an embodiment one of said first antibody or said second antibody is a polyclonal antibody. In an embodiment one of said first antibody or said second antibody is an avian, rabbit or goat polyclonal antibody which is capable of binding to UspA2. In an embodiment one of said first antibody or said second antibody is a rabbit-polyclonal antibody which binds to UspA2. In an embodiment said second antibody is a rabbit-polyclonal antibody which binds to UspA2.

In an embodiment said rabbit-polyclonal antibody which binds to UspA2, binds at an epitope within the region A500-L551 of SEQ ID NO: 1. In an embodiment said rabbit-polyclonal antibody which binds to UspA2, binds at an epitope within the region A471-L522 of SEQ ID NO: 75 (i.e. MC-009). In an embodiment said rabbit-polyclonal antibody which binds to UspA2, binds at an epitope within the sequence of SEQ ID NO: 81.

In an embodiment, one of said first antibody or said second antibody is a capture antibody and is immobilized on a solid support. In an embodiment one of said first antibody or said second antibody is a detection antibody and is not immobilized on a solid support. Reference to capture/detection antibodies may also relate to capture/detection antigen binding proteins.

In an embodiment;
a) said first antibody is the monoclonal antibody comprising a VH region of SEQ ID NO: 94 and a VL region of SEQ ID NO: 96 and is immobilized on a solid support and said second antibody is a polyclonal antibody which binds to UspA2 and is not immobilized on a solid support; or;
b) said first antibody is a polyclonal antibody which binds to UspA2 and is immobilized on a solid support and said second antibody is the monoclonal antibody comprising a VH region comprising a VH region of SEQ ID NO: 94 and a VL region of SEQ ID NO: 96 and is not immobilized on a solid support.

In an embodiment;
a) said first antibody is the monoclonal antibody comprising a VH region comprising a VH region of SEQ ID NO: 94 and a VL region of SEQ ID NO: 96 and is immobilized on a solid support and said second antibody is a rabbit polyclonal antibody which binds to UspA2 at an epitope within the region A471-L522 of the UspA2 protein construct of SEQ ID NO: 75 (MC-009) and is not immobilized on a solid support; or;
b) said first antibody is a rabbit polyclonal antibody which binds to UspA2 at an epitope within the region A471-L522 of the UspA2 protein construct of SEQ ID NO: 75 (MC-009) and is immobilized on a solid support and said second antibody is the monoclonal antibody comprising a VH region comprising a VH region of SEQ ID NO: 94 and a VL region of SEQ ID NO: 96 and is not immobilized on a solid support.

In an embodiment, said first antibody comprises a VH region of SEQ ID NO: 94 and a VL region of SEQ ID NO: 96 and is immobilized on a solid support and said second antibody is a polyclonal antibody which binds to UspA2 and is not immobilized on a solid support, optionally a rabbit polyclonal antibody which binds to UspA2. In an embodiment said first antibody comprises a VH region of SEQ ID NO: 94 and a VL region of SEQ ID NO: 96, binds to UspA2 at an epitope within a consensus sequence of YNELQD-[A/Q]-YA-[QK/KQ]-QTE (SEQ ID NO: 82), e.g. SEQ ID NO: 83, 84, 85 or 86 and is immobilized on a solid support and said second antibody is a rabbit polyclonal antibody which binds to UspA2 and is not immobilized on a solid support. In an embodiment said first antibody comprises a VH region of SEQ ID NO: 94 and a VL region of SEQ ID NO: 96, binds to UspA2 at an epitope within a consensus sequence of YNELQD-[A/Q]-YA-[QK/KQ]-QTE (SEQ ID NO: 82), e.g. SEQ ID NO: 83, 84, 85 or 86 and is immobilized on a solid support and said second antibody is a rabbit polyclonal which binds to UspA2 at one or more of amino acid residues within the region A471-L522 of the UspA2 protein construct of SEQ ID NO: 75 (MC-009) and is not immobilized on a solid support.

In an embodiment, the assay of the invention further comprises a blocking step. During an ELISA it may be desirable to add a blocking reagent and/or detergent e.g. to reduce non-specific binding interactions which might distort the assay's results. Blocking procedures are familiar to people working in the ELISA field. In an embodiment said blocking step is conducted using a blocking buffer. In an embodiment said blocking buffer comprises bovine serum albumin (BSA), non-fat dry milk or casein, whole normal serum, fish gelatin, polyethylene glycol (PEG), polyvinyl alcohol (PVA) or polyvinylpyrrolidone (PVP).

Labelling of antibodies in an ELISA can take various forms. In an ELISA an antibody (e.g. detection antibody) can be labelled with an enzyme, which is then used to catalyze a reaction whose product is readily detectable. The linked enzyme can cause a detectable change in an enzyme substrate which is added to the labelled antibody after it has bound its target epitope e.g. to modify a substrate in a manner which causes a colour change. For example, the enzyme may be a peroxidase (e.g. horseradish peroxidase, HRP), or a phosphatase (e.g. alkaline phosphatase, AP). Other enzymes can also be used e.g. laccase, β-galactosidase, etc.

The choice of substrate will depend on the choice of linked enzyme. Preferred substrates undergo a colorimetric change, a chemiluminescent change, or a chemifluorescent change when contacted with the linked enzyme. Colorimetric substrates (and their enzymatic partners) include but are not limited to: PNPP or p-Nitrophenyl Phosphate (AP); ABTS or 2,2′-Azinobis [3-ethylbenzothiazoline-6-sulfonic acid] (HRP); OPD or o-phenylenediamine dihydrochloride (HRP); and TMB or 3,3′,5,5′-tetramethylbenzidine (HRP). Chemiluminescent substrates include luminol or 5-amino-2,3-dihydro-1,4-phthalazinedione (HRP), particularly in the presence of modified phenols such as p-iodophenol. Chemifluorescent substrates include p-hydroxyhydrocinnamic acid. Various proprietary substrates are also available, and these can be used with the invention if desired e.g. QuantaBlu, QuantaRed, SuperSignal, Turbo TMB, etc.

In an embodiment, said second antibody that is not immobilized on said solid support is labelled (for example is enzyme-labelled). In an embodiment said labelled second antibody is a rabbit polyclonal antibody which binds to UspA2 at one or more of amino acid residues within the region A471-L522 of the UspA2 protein construct of SEQ ID NO: 75 (MC-009) and is not immobilized on a solid support.

In an embodiment said second antibody is conjugated to a high affinity tag such as biotin, avidin or streptavidin. An enzyme conjugated to a ligand for the tag, such as avidin, streptavidin or biotin can then be used to detect immobilized primary antibody. Any of these variations can be used within the scope of the overall invention.

In an embodiment said second antibody is labelled with biotin (i.e. is biotinylated). As used herein the term “biotinylated” or “labelled with biotin” refers to a protein, nucleic acid or other molecule (e.g. antibody or secondary antibody) which has undergone a process wherein biotin is covalently attached to it. Biotin can be bound by avidins and streptavidin with high affinity. Streptavidin can be conjugated to a detection system (e.g. peroxidase-conjugated streptavidin) enabling quantification of bound antibody. For example, peroxidase-conjugated streptavidin binds to a biotinylated antibody and the conjugated peroxidase (e.g. horseradish peroxidase) provides enzyme activity for detection using an appropriate substrate system.

In an embodiment said second antibody that is labelled with biotin is conjugated with peroxidase conjugated streptavidin.

As an alternative to using a conjugated enzyme as the label, other labelling is possible. For instance, other indirect labels {i.e. alternative to enzymes) can be used, but it is also possible to label the antibody by conjugation to a direct label such as a coloured particle, an electrochemically active reagent, a redox reagent, a radioactive isotope, a fluorescent label or a luminescent label.

In an embodiment said second antibody that is not immobilized on said solid support is unlabeled. In an embodiment said unlabeled second antibody is a rabbit polyclonal antibody which binds to UspA2 at one or more of amino acid residues within the region A471-L522 of the UspA2 protein construct of SEQ ID NO: 75 (MC-009) and is not immobilized on a solid support.

In an embodiment, said unlabeled second antibody is contacted with an enzyme-labelled third antibody that binds to said unlabeled second antibody to detect the formation of the first antibody-UspA2-second antibody complex. In an embodiment the enzyme-labelled third antibody binds to the polyclonal antibody which binds to UspA2. In an embodiment the enzyme-labelled third antibody binds to the rabbit polyclonal antibody which binds to UspA2 at one or more of amino acid residues within the region A471-L522 of the UspA2 protein construct of SEQ ID NO: 75 (MC-009).

In an embodiment said enzyme-labelled third antibody is a peroxidase labelled third antibody. In an embodiment said enzyme-labelled third antibody is a peroxidase labelled anti-rabbit antibody, optionally a peroxidase labelled goat anti-rabbit secondary antibody. In an embodiment said enzyme-labelled third antibody is added to the reaction once the first antibody-UspA2-second antibody complexes have already formed. After binding to the second antibody, conversion of a substrate into a detectable product by the enzyme which the third antibody is labelled with, detects the formation of the first antibody-UspA2-second antibody complexes.

In an embodiment, the formation of the first antibody-UspA2-second antibody complex is determined by measuring the conversion of a substrate into a detectable product. In an embodiment, the conversion of a substrate into a detectable product is catalysed by peroxidase. In an embodiment, the formation of the first antibody-UspA2-second antibody complex is determined by measuring the conversion of a substrate into a detectable product catalysed by the peroxidase enzyme conjugated to the third antibody.

In an embodiment, the conversion of a substrate into a detectable product is determined by measuring a change in absorbance, chemiluminescence or fluorescence. In an embodiment, the conversion of a substrate into a detectable product by peroxidase, is determined by measuring a change in absorbance.

In an embodiment the substrate is o-phenylenediamine dihydrochloride (OPD), 3,3′,5,5′-tetramethylbenzidine (TMB), 3,3′-diaminobenzidine (DAB) or 2,2′-azino-bis(3-ethylbezothiazoline-6-sulphonic acid (ABTS), AmplexRed, Luminol, Homovanillic acid or (3-Amino-9-Ethylcarbazole (AEC). In an embodiment, the substrate is o-phenylenediamine dihydrochloride (OPD).

In an embodiment the substrate is added to the reaction as a solution of revelation. In an embodiment the solution of revelation comprises OPD, H₂O₂ and citrate. In an embodiment the solution of revelation comprises 2.2 mM of OPDA and 5 μl of H₂O₂ in 10 ml of citrate 0.1M pH 4.5 buffer.

In an embodiment the detectable product is 2,3-Diaminophenazine.

In an embodiment, the reaction which converts of o-phenylenediamine dihydrochloride (OPD) into 2,3-Diaminophenazine by peroxidase takes place at room temperature. In an embodiment, the reaction which converts o-phenylenediamine dihydrochloride (OPD) into 2,3-Diaminophenazine by peroxidase takes place at between 2-8° C. In an embodiment, the reaction which converts of o-phenylenediamine dihydrochloride (OPD) into 2,3-Diaminophenazine by peroxidase takes place in less than 30 hours, less than 24 hours, less than 18 hours, less than 15 hours, less than 10 hours, less than 4 hours or less than 1 hour. In an embodiment the reaction which converts of o-phenylenediamine dihydrochloride (OPD) into 2,3-Diaminophenazine by peroxidase takes place in between 0 and 60 minutes, 5 and 45 minutes, 10 and 30 minutes or 10 and 25 minutes. In an embodiment, the reaction which converts of o-phenylenediamine dihydrochloride (OPD) into 2,3-Diaminophenazine by peroxidase takes place in 15 minutes. In an embodiment the reaction takes place in the dark.

In an embodiment the reaction which converts of o-phenylenediamine dihydrochloride (OPD) into 2,3-Diaminophenazine by peroxidase is stopped prior to measuring the change in absorbance. In an embodiment the reaction is stopped with hydrochloric acid (HCl) 1N, or sulfuric acid (H₂SO₄) 1N.

In an embodiment, conversion of o-phenylenediamine dihydrochloride (OPD) into 2,3-Diaminophenazine by peroxidase is determined by measuring a change in absorbance, optionally at between 350 nm and 650 nm (e.g. 450 nm, 490 nm etc). In an embodiment, conversion of o-phenylenediamine dihydrochloride (OPD) into 2,3-Diaminophenazine by peroxidase is determined by measuring a change in absorbance at 490 nm±10%.

It is foreseen that any suitable detection system could be utilised, in order to quantify the ELISA. For example, any chromogenic, chemiluminescent, or fluorescent readout from the enzyme-substrate interaction or excited fluorophore could be utilised.

In a further aspect of the invention there are provided functional antibodies for use in an in vitro relative potency (IVRP) assay. Said IVRP assay is used in QC batch testing to ensure that vaccines comprising UspA2 are suitable for release to the public.

In an embodiment the assay of the invention further comprises comparing the formation of the first antibody-UspA2-second antibody complexes formed with a test sample comprising UspA2 with the formation of the first antibody-UspA2-second antibody complexes formed with a reference sample. In an embodiment the assay of the invention further comprises comparing the amount of first antibody-UspA2-second antibody complexes formed with a test sample with the amount of first antibody-UspA2-second antibody complexes formed with a reference sample. In an embodiment the reference sample is an UspA2 protein construct (e.g. MC-009 of SEQ ID NO: 75) which has been tested in human. In an embodiment the reference sample is an UspA2 protein construct (e.g. MC-009 of SEQ ID NO: 75) which has demonstrated clinical efficacy in human. In an embodiment the reference sample is diluted during or prior to conducting the assay of the invention.

In an embodiment the assay of the invention further comprises comparing the change in absorbance caused by enzymatic conversion of a substrate into a detectable product (and thus the amount of peroxidase labelled third antibody bound to second antibody which is itself in the first antibody-UspA2-second antibody complexes) using a test sample comprising UspA2 with the change in absorbance using a reference sample.

In an embodiment, the assay of the invention is used to determine or measure the presence of UspA2 in its native conformation. In an embodiment the assay of the invention is used to determine or measure the potency of a test sample comprising UspA2. In an embodiment, the assay of the invention involves both the monoclonal and polyclonal antibodies described herein binding to conformationally sensitive epitopes on the surface of UspA2 (for example the UspA2 protein construct of SEQ ID NO: 75, i.e. MC-009). Said epitopes are conformationally sensitive since, changes in the conformation of UspA2 impacts antibody binding to said epitopes. Said epitopes are known to be biologically relevant since both antibodies are cross bactericidal against heterologous strains of M. catarrhalis. The presence of such epitopes in the vaccine is expected to elicit protective antibodies in immunized patients. The assay of the invention is therefore believed to be predictive of clinical potency, and reduced binding of the antibodies (i.e. reduced formation of the first antibody-UspA2-second antibody complexes) reflects a sub-potent vaccine. In an embodiment the assay of the invention is predictive of clinical potency.

In an embodiment the test sample comprising UspA2 comprises a sequence with at least 80% identity (e.g. at least 85% identity, at least 90% identity, at least 95% identity) to any one of SEQ ID NO: 59 (MC-001), SEQ ID NO: 61 (MC-002), SEQ ID NO: 63 (MC-003), SEQ ID NO: 65 (MC-004), SEQ ID NO: 67 (MC-005), SEQ ID NO: 69 (MC-006), SEQ ID NO: 71 (MC-007), SEQ ID NO: 73 (MC-008), SEQ ID NO: 75 (MC-009), SEQ ID NO: 77 (MC-010) or SEQ ID NO: 79 (MC-011). In an embodiment the test sample comprising UspA2 is SEQ ID NO: 75 (MC-009). In an embodiment, the test sample comprising UspA2 (e.g. MC-009) is diluted during or prior to conducting the assay of the invention.

In a further aspect there is provided a binding assay for in vitro analysis of a Moraxella catarrhalis antigen vaccine sample from a batch of final vaccine in the form in which it would be released to the public comprising the steps of:
i) permitting the M. catarrhalis protein antigen within the vaccine sample to interact with an antibody of the invention (i.e. the UspA2 monoclonal antibody comprising VH region comprising a VH region of SEQ ID NO: 94 and a VL region of SEQ ID NO: 96) which either a) is cross-bactericidal against heterologous strains of M. catarrhalis, or b) recognises an epitope in the UspA2 antigen; then
ii) measuring the interaction between the M. catarrhalis antigen and antibody from step (i) wherein the binding assay is an ELISA (optionally a sandwich ELISA).

In a further aspect there is provided a binding assay for in vitro analysis of a vaccine sample comprising UspA2 from a batch of final vaccine in the form in which it would be released to the public comprising the steps of:
i) permitting the UspA2 protein antigen within the vaccine sample to interact with an antibody of the invention (i.e. the UspA2 monoclonal antibody comprising VH region comprising a VH region of SEQ ID NO: 94 and a VL region of SEQ ID NO: 96) which either a) is cross-bactericidal against heterologous strains of M. catarrhalis, or b) recognises an epitope in the UspA2 antigen; then
ii) measuring the interaction between the UspA2 protein antigen and antibody from step (i) wherein the binding assay is an ELISA (optionally a sandwich ELISA).

In an embodiment the sample is analysed in the form in which it is taken from the batch, either at full strength or after dilution. In an embodiment said vaccine is the protein construct MC-009 of SEQ ID NO: 75.

In addition to the ELISA formats discussed above, the invention can also be extended to use alternatives to ELISA, such as flow injection immunoaffinity analysis (FIIAA), AlphaLISA or AlphaScreen, dissociation-enhanced lanthanide fluorescent immunoassay (DELFIA), ELAST, the BIO-PLEX Suspension Array System, MSD, etc. Any suitable antibody-antigen complex binding assays can be used.

For example, in an embodiment the assay of the invention may be carried out using an automated immunoassay, for example using the GYROLAB system. The GYROLAB system is a fully automated nanoliter-scale immunoassay platform containing streptavidin-coated microfluidic columns in a compact-disc (CD) technology format. The GYROLAB Bioaffy CD contains 96 to 112 streptavidin-coated columns inside microstructures. Sequential addition of reagents and samples in each microstructure is fully automated. Added capture reagent is first stopped by hydrophobic breaks and centrifugal force due to the rotation of the CD drives reagent into colums and it binds to streptavidin-coated particles. Samples and detection reagent are then applied to activated columns and immuno-sandwiches are assembled. The GYROLAB system, including preparation of its microfluidic affinity columns is described on the www.gyros.com website.

The IVRP assay of the invention may be carried out using the GYROLAB system (i.e UspA2 IVRP GYROLAB assay). The UspA2 IVRP GYROLAB assay uses an anti-Uspa2 monoclonal antibody (mAb FHUSPA2/10) as the capture antibody and a rabbit anti-UspA2 polyclonal antibody as the detection antibody.

The UspA2 IVRP GYROLAB assay substantially comprises the following steps:
i) All reagents, reference standard and samples are diluted to defined working concentrations.
ii) The GYROLAB Bioaffy 1000 CD contains 96 streptavidin-coated columns inside microstructures. Sequential addition of reagents and samples in each microstructure is fully automated and performed according to a Bioaffy 1000 CD slow analyte spin 3-step (capture-analyte-detection) wizard method (1000-3W-005-Wash 2). Added biotinylated mAb FHUSPA2/10 is first stopped by hydrophobic breaks. Then, centrifugal force generated by the rotation of CD drives reagent into columns which binds to streptavidin-coated particles.
iii) After column washing, reference standard and samples are then applied to activated columns.
iv) Alexa Fluor647-labelled anti-UspA2 polyclonal antibody is then added to detect captured analyte and fluorescence is read by the laser. Intensity of each sample is calculated using a four logistic parameters curve to the standard curve.

In an embodiment there is therefore provided, an assay to determine potency with respect to UspA2 using the UspA2 monoclonal antibody comprising VH region comprising a VH region of SEQ ID NO: 94 and a VL region of SEQ ID NO: 96, wherein the assay is a sandwich ELISA assay and wherein the sandwich ELISA assay is conducted using the GYROLAB system.

In an embodiment the assay of the invention is conducted using an automated immunoassay, for example using the GYROLAB system. In an embodiment the relative potency of a PE-PilA test antigen and the relative potency of a Protein D antigen is measured simultaneously to the relative potency of the UspA2 antigen (for example SEQ ID NO:75) (i.e. on the same GYROLAB CD).

### Kits

The invention further provides kits for use in assaying the potency of a test antigen (e.g. for assaying the potency of a sample comprising UspA2). There is provided a kit to (i) detect, measure the levels of, and/or measure a change in the conformation of a test antigen or (ii) determine potency of a test antigen, comprising: reagents for preparing an assay mixture, at least one antibody which binds to UspA2, and optionally instructions for use thereof.

In an embodiment said kit comprises two antibodies which bind to UspA2. In an embodiment the kit comprises a) the monoclonal antibody comprising a VH region comprising a VH region of SEQ ID NO: 94 and a VL region of SEQ ID NO: 96 and b) a rabbit polyclonal antibody which binds to UspA2 at one or more of amino acid residues within the region A471-L522 of the UspA2 protein construct of SEQ ID NO: 75 (MC-009).

In an embodiment at least one antibody which binds to UspA2 is immobilized on a solid support. In an embodiment the antibody which is immobilized on a solid support comprises a VH region comprising a VH region of SEQ ID NO: 94 and a VL region of SEQ ID NO: 96. In an embodiment, said monoclonal antibody comprising a VH region comprising a VH region of SEQ ID NO: 94 and a VL region of SEQ ID NO: 96 is pre-coated into the wells of a microtiter plate when the kit is supplied.

In an embodiment the kits comprise all reagents and materials required in order (i) to detect, measure the levels of, and/or measure a change in conformation of a test antigen or (ii) determine potency of a test antigen. Alternatively, there is provided kits which comprise a subset of the reagents and materials required in order (i) to detect, measure the levels of, and/or measure a change in conformation of a test antigen or (ii) determine potency of a test antigen (for example wherein the kit comprises all essential buffers, reagents and consumables but does not comprise instrumentation, devices, probes etc). In an embodiment, the kit further comprises instructions for use.

The invention thus also provides a kit which is used (i) to detect, measure the levels of, and/or measure a change in conformation of a test antigen or (ii) to determine potency of a test antigen.

The kit includes containers for storing reagents prior to use. Each reagent may have its own container, or several reagents may be pre-mixed and packaged together in a container.

The testing device is preferably a multi-well microtiter plate (e.g., 96 well microtiter plate), but can also be any type of receptacle such as petri dishes or plates with a plurality of wells in which an assay can be conducted. The reagents may be disposed in the wells of the testing device, although it will be appreciated that such reagents can instead be dispensed in the wells of the testing device by the end user just prior to conducting the assay. The kit may further include a set of instructions for using the kit in an assay. The kit may optionally be supplied frozen, suitable for storage at 2-8° C. or may be supplied at room temperature. In an embodiment the kit may be supplied in different components, each with different storage requirements. In an embodiment components of the kit may be supplied in lyophilized or biotinylated form and may require resuspension by the end-user prior to conducting the assay of the invention. In an embodiment the components of the kit are supplied sterile.

In an embodiment the kit requires the end user to dilute their test antigen prior to use (optionally 2-fold, optionally 10-fold, optionally 50-fold, optionally 100-fold, optionally 1000-fold, optionally 10,000-fold or greater). In an embodiment the kit further comprises a reference or internal standard which may be used to compare against the response observed with the test antigen.

The kit of the invention may further comprise an expiration date, after which the integrity of the kit can no longer be assured.

### Method for Analysis

The invention further provides a method for in vitro analysis of a test antigen, comprising steps of: (i) performing the assay of the invention on a test antigen and a reference sample of known potency; and (ii) comparing the results from step (i) to determine the potency of the test antigen relative to the reference sample. In an embodiment, the invention further provides a method for in vitro analysis of a test sample comprising an UspA2 antigen (e.g. MC-009), comprising steps of: (i) performing the assay of the invention on a test sample comprising an UspA2 antigen and a reference sample of known potency; and (ii) comparing the results from step (i) to determine the potency of the UspA2 test antigen relative to the reference samples.

In an embodiment there is provided a method for analysing a batch of vaccine, comprising steps of: (i) assaying a test antigen taken from a batch of vaccine by the method of the invention and, if the results of step (i) indicate an acceptable relative potency, (ii) releasing further vaccines from the batch for in vivo use. In an embodiment the method of the invention is carried out in duplicate, triplicate or more. In an embodiment an acceptable relative potency will be demonstrated when the test antigen is within the specification limits of the assay, as compared to the reference sample, wherein the specification limit is set as approximately 75%-125% of the reference sample.

In an embodiment an acceptable relative potency will be achieved when the ED₅₀ of the test antigen is above a threshold limit. In an embodiment an acceptable relative potency will be achieved when no statistically significant difference is observed between the data of the test antigen compared to the data of the reference sample. In an embodiment, the test antigen will fail is an acceptable relative potency is not achieved. In an embodiment, a test antigen which fails the assay of the invention will not be released to the public.

In an embodiment the test antigen will be diluted prior to or during the assay of the invention. In an embodiment the test antigen will be diluted optionally 2-fold, optionally 10-fold, optionally 50-fold, optionally 100-fold, optionally 1000-fold, optionally 10,000-fold or greater.

Embodiments of the invention are further described in the subsequent numbered paragraphs:
1. The use of an antibody which binds to UspA2 in an assay for the detection of, or measurement of a change in, the conformation of UspA2 wherein UspA2 is present in a test sample.
2. The use according to paragraph 1 wherein the antibody which binds to UspA2 is a monoclonal antibody, optionally an IgG2A mouse monoclonal antibody.
3. The use according to paragraphs 1 and 2 wherein the antibody which binds to UspA2 comprises a VH region comprising a sequence at least 80% identical to SEQ ID NO: 94 and a VL region comprising a sequence at least 80% identical to SEQ ID NO: 96.
4. The use according to paragraphs 1-3 wherein the antibody which binds to UspA2 comprises a VH region of SEQ ID NO: 94 and a VL region of SEQ ID NO: 96.
5. The use according to paragraphs 1-4 wherein the antibody which binds to UspA2 comprises any one or a combination of CDRs selected from (i) CDRH1 (SEQ ID NO: 87), CDRH2 (SEQ ID NO: 88), CDRH3 (SEQ ID NO: 89), CDRL1 (SEQ ID NO: 90), CDRL2 (SEQ ID NO: 91) and CDRH3 (SEQ ID NO: 92) or (ii) a CDR variant of (i), wherein the variant has 1, 2, or 3 amino acid modifications in each CDR, which is able to bind to an epitope within the consensus sequence of SEQ ID NO: 82.
6. The use according to any of paragraphs 1-5 wherein the antibody which binds to UspA2 binds an epitope within the consensus sequence of SEQ ID NO: 82 (e.g. SEQ ID NO: 83, 84, 85, 86)
7. The use according to any of paragraphs 1-6 wherein the UspA2 which is present in a test sample comprises a sequence with at least 80% identity (e.g. at least 85% identity, at least 90% identity, at least 95% identity) to any one of SEQ ID NO: 59 (MC-001), SEQ ID NO: 61 (MC-002), SEQ ID NO: 63 (MC-003), SEQ ID NO: 65 (MC-004), SEQ ID NO: 67 (MC-005), SEQ ID NO: 69 (MC-006), SEQ ID NO: 71 (MC-007), SEQ ID NO: 73 (MC-008), SEQ ID NO: 75 (MC-009), SEQ ID NO: 77 (MC-010 or SEQ ID NO: 79 (MC-011).
8. The use according to any of paragraphs 1-7 wherein the UspA2 which is present in a test sample is UspA2 of SEQ ID NO: 75 (MC-009)
9. An assay comprising the steps of contacting a test sample comprising UspA2 with a first antibody and a second antibody to form a first antibody-UspA2-second antibody complex and detecting or measuring the amount of said first antibody-UspA2-second antibody complex.
10. The assay according to paragraph 9 which is a sandwich ELISA.
11. The assay according to paragraph 9 or paragraph 10, wherein at least one of said first antibody or said second antibody is immobilized on a solid support.
12. The assay according to paragraphs 9-11 wherein said first antibody and said second antibody bind non-overlapping epitopes on UspA2.
13. The assay according to paragraphs 9-12 wherein at least one of said first antibody or said second antibody is a monoclonal antibody.
14. The assay according to paragraph 13 wherein at least one of said first antibody or said second antibody comprises a VH region comprising a sequence at least 80% identical to SEQ ID NO: 94 and a VL region comprising a sequence at least 80% identical to SEQ ID NO: 96.
15. The assay according to paragraph 13-14 wherein at least one of said first antibody or said second antibody comprises a VH region of SEQ ID NO: 94 and a VL region of SEQ ID NO: 96.
16. The assay according to paragraphs 13-15 wherein at least one of said first antibody or said second antibody comprises any one or a combination of CDRs selected from (i) CDRH1 (SEQ ID NO: 87), CDRH2 (SEQ ID NO: 88), CDRH3 (SEQ ID NO: 89), CDRL1 (SEQ ID NO: 90), CDRL2 (SEQ ID NO: 91) and CDRH3 (SEQ ID NO: 92) or (ii) a CDR variant of (i), wherein the variant has 1, 2, or 3 amino acid modifications in each CDR, which is able to bind to an epitope within the consensus sequence of SEQ ID NO: 82.
17. The assay according to paragraphs 9-12 wherein at least one of said first antibody or said second antibody is a polyclonal antibody.
18. The assay according to paragraph 17 wherein at least one of said first antibody or said second antibody is an avian, rabbit or goat polyclonal antibody which is capable of binding to UspA2.
19. The assay according to any of paragraph 9-18 wherein a) said first antibody is the monoclonal antibody as described in paragraphs 13-16 and is immobilized on a solid support and said second antibody is a polyclonal antibody and is not immobilized on a solid support; or b) said first antibody is a polyclonal antibody and is immobilized on a solid support and said second antibody is the monoclonal antibody as described in paragraphs 13-16 and is not immobilized on a solid support.
20. The assay according to any of paragraphs 9-19 wherein said first antibody comprises a VH region of SEQ ID NO: 94 and a VL region of SEQ ID NO: 96 and is immobilized on a solid support and said second antibody is a polyclonal antibody which binds to UspA2 and is not immobilized on a solid support, optionally a rabbit polyclonal antibody.
21. The assay according to paragraphs 19-20 wherein said second antibody that is not immobilized on said solid support is labelled.
22. The assay according to paragraph 21 wherein said second antibody that is not immobilized on said solid support is labelled with biotin (i.e. is biotinylated).
23. The assay according to paragraph 22 wherein said second antibody is labelled with biotin and is conjugated with peroxidase conjugated streptavidin.
24. The assay according to paragraphs 19-20 wherein said second antibody that is not immobilized on said solid support is unlabeled.
25. The assay according to paragraph 24, wherein said unlabeled second antibody is contacted with an enzyme-labelled third antibody that binds to said unlabeled second antibody to detect the formation of the first antibody-UspA2-second antibody complex.
26. The assay according to paragraph 24 or paragraph 25 wherein the enzyme-labelled third antibody binds to the polyclonal antibody which is capable of binding UspA2.
27. The assay according to paragraphs 24-26 wherein said enzyme-labelled third antibody is peroxidase labelled.
28. The assay according to paragraph 24-27 wherein said enzyme-labelled third antibody is a peroxidase labelled anti-rabbit antibody, optionally a peroxidase labelled goat anti-rabbit secondary antibody.
29. The assay of any of paragraphs 9-28 wherein the formation of the first antibody-UspA2-second antibody complex is determined by measuring the conversion of a substrate into a detectable product.
30. The assay of paragraph 29 wherein the conversion of a substrate into a detectable product is catalysed by peroxidase.
31. The assay of paragraph 30 wherein the conversion of a substrate into a detectable product by peroxidase, is determined by measuring a change in absorbance, chemiluminescence or fluorescence.
32. The assay of paragraphs 29-31 wherein the substrate is o-phenylenediamine dihydrochloride (OPD), 3,3′,5,5′-tetramethylbenzidine (TMB), 3,3′-diaminobenzidine (DAB) or 2,2′-azino-bis(3-ethylbezothiazoline-6-sulphonic acid (ABTS), AmplexRed, Luminol, Homovanillic acid or (3-Amino-9-Ethylcarbazole (AEC).
33. The assay of paragraph 32 wherein the substrate is o-phenylenediamine dihydrochloride (OPD).
34. The assay of paragraphs 29-31 wherein the product is 2,3-Diaminophenazine
35. The assay of paragraph 34 wherein conversion of o-phenylenediamine dihydrochloride (OPD) into 2,3-Diaminophenazine by peroxidase is determined by measuring a change in absorbance, optionally at between 350 nm and 650 nm (e.g. 450 nm, 490 nm etc).
36. The assay of any of paragraphs 9-35 further comprising comparing the amount of the first antibody-UspA2-second antibody complexes formed with a test sample comprising UspA2 with the amount of the first antibody-UspA2-second antibody complexes formed with a reference sample.
37. The assay of any of paragraphs 9-36 for use in determining or measuring the presence of UspA2 in its native conformation.
38. The assay of any of paragraph 9-37 to determine or measure the potency of a test sample comprising UspA2.
39. The assay of any of paragraphs 9-36 wherein the test sample comprising UspA2 comprises an UspA2 antigen with a sequence with at least 80% identity (e.g. at least 85% identity, at least 90% identity, at least 95% identity) to any one of SEQ ID NO: 59 (MC-001), SEQ ID NO: 61 (MC-002), SEQ ID NO: 63 (MC-003), SEQ ID NO: 65 (MC-004), SEQ ID NO: 67 (MC-005), SEQ ID NO: 69 (MC-006), SEQ ID NO: 71 (MC-007), SEQ ID NO: 73 (MC-008), SEQ ID NO: 75 (MC-009), SEQ ID NO: 77 (MC-010 or SEQ ID NO: 79 (MC-011).
40. The assay of paragraph 39 wherein the test sample comprising UspA2 comprises UspA2 of SEQ ID NO: 75.
41. The assay according to any one of paragraphs 9-40 wherein the assay is performed using an automated immunoassay, for example using the GYROLAB system.
42. A kit to (i) detect, measure the levels of, and/or measure a change in the conformation of a test antigen or (ii) determine potency of a test antigen, comprising: reagents for preparing an assay mixture, at least one antibody which binds to UspA2, and optionally instructions for use thereof.
43. A kit according to paragraph 42 wherein at least one antibody which binds to UspA2 is immobilized on a solid support.
44. A kit according to paragraph 43 wherein the antibody which is immobilized on a solid support comprises a VH region of SEQ ID NO: 94 and a VL region of SEQ ID NO: 96.
45. A method for in vitro analysis of a test antigen, comprising steps of: (i) performing the assay of any of paragraphs 9-41 on a test antigen and a reference sample of known potency; and (ii) comparing the results from step (i) to determine the potency of the test antigen relative to the reference sample.
46. A method for analysing a batch of vaccine, comprising steps of: (i) assaying a test antigen taken from a batch of vaccine by the method of paragraph 45; and, if the results of step (i) indicate an acceptable relative potency, (ii) releasing vaccine from the batch for in vivo use.

In order that this invention may be better understood, the following examples are set forth. These examples are for purposes of illustration only and are not to be construed as limiting the scope of the invention in any manner.

### EXAMPLES

### Example 1: FHUSPA2/10 Monoclonal Antibody Sequencing

Aim: To obtain the nucleic and amino acid sequence of hybridoma-secreted antibody of FHUSPA2-clone. The whole procedure aimed to sequence exclusively the variable regions of the light and heavy antibody chains (VL and VH). The sequencing strategy was designed to also obtain the sequence of a small region of the constant region (˜50-60 bp) for confirmation of the antibody class/subtype
Methods: The whole procedure can be summarized as follows:
1. Thawing and growth of hybridoma cell clone
2. RNA extraction
3. cDNA generation by retro-transcription
4. 3′ polyA tailing
5. 5′ Rapid Amplification of cDNA Ends (RACE) PCR
6. Cloning into commercial plasmid (TOPO PCR cloning)
7. Colony picking, bacterial growth and plasmid extraction
8. Sanger sequencing
   Briefly, hybridoma cells were thawed and grown for 10 days;
   Thawing of cells: 15 ml Falcon tubes were prepared containing 10 ml of warm DMEM medium. Cells were thawed by placing the cryotube rapidly in a 37° C. water bath.
   Cells were transferred into the Falcon tube and centrifuged for 10 minutes at 1000 rpm.
   The supernatant was carefully poured away, and the cells were resuspended with 10 ml of warm D-MEM and re-centrifuged for 10 minutes at 1000 rpm. In the meanwhile, 1 ml of thawing medium was transferred into each well of the first row of a 24-well plate.
   The supernatant was again poured away, and the cells were resuspended with 1 ml of warm thawing media. The resuspension was transferred into the first well, mixed by gentle pipetting and then 1 ml was transferred into the near well. This 1:1 dilution was continued until the last well. 1 ml of media was added to each well, to reach 2 ml of cell culture in each well.
   The plate was placed in an incubator at 37° C. with a 5% CO2 atmosphere.
   After few days, the cells were transferred from the well where they are not fully convergent into a T25 flask for adherent cells adding fresh thawing media to 10 ml total volume.

### Cell Subculturing Protocol

Cells were recovered by shaking gently the T25 flask and pouring the resuspension into a 50 ml Falcon tube. The tube was then centrifuged for 10 minutes at 1000 rpm, and the pellet resuspend in warm D-MEM. This process was repeated however the second resuspension step was conducted in a 50 ml of warm growing medium. The cells were then transferred into a T75 flask.

RNA was then extracted (4 samples of cells, each containing 7×10⁶ cells) using the Qiagen RNeasy Mini kit (according to manufacturer's instructions) followed by cDNA generation by retro transcription of 4.5 μg RNA. Retro transcription was performed using Superscript IV first-strand synthesis system (Invitrogen) and a set of oligos specific for either the light chain or heavy chain amplification:

HEAVY CHAIN oligos (for all isotypes):
RT-mHingeG1_rev
(SEQ ID NO: 98)
5′-gcaaggcttacaaccacaatc-3′
RT-mHingeG2a_rev
(SEQ ID NO: 99)
5′-gaggacagggcttgattgtgg-3′
RT-mHingeG2b_rev
(SEQ ID NO: 100)
5′-gaggacaggggttgattgttg-3′
RT-mHingeG2c_rev
(SEQ ID NO: 101)
5′-ggacaggggttctgtgttatgg-3′
RT-mHingeG3_rev
(SEQ ID NO: 102)
5′-ctgggcttgggtattctagg-3′
LIGHT CHAIN oligos (for both kappa and lambda
classes):
RT_mKappaCL_rev
(SEQ ID NO: 103)
5′-ctcattcctgttgaagctcttg-3′
RT_mLambda1/4-CL_rev
(SEQ ID NO: 104)
5′-gcacgggacaaactcttctc-3′
RT_mLambda2/3-CL_rev
(SEQ ID NO: 105)
5′-ctgcaggagacagactcttctc-3′

3′ polyA tailing was performed using between 680 and 200 ng of cDNA and Terminal Deoxynucleotidyl Transferase (ThermoScientific) and dATP (Invitrogen). This generated (after column purification) 400-800ng of polyA cDNA, following which 5′ rapid amplification of cDNA ends (RACE) PCR was performed using either Q5 Hot Start polymerase (NEB) or Platinum SuperFi polymerase (Invitrogen), and a set of oligos specific for either the light chain or heavy chain amplification:

HEAVY CHAIN oligos (for all isotypes):
TAR2_RACE-5E_mG1_rev
(SEQ ID NO: 106)
5′-GTCTCGTGGGCTCGGAGATGTGTATAAGAGACAGgttagtttgggc
agcagatc-3′
TAR2_RACE-5E_mG2a.2c_rev
(SEQ ID NO: 107)
5′-GTCTCGTGGGCTCGGAGATGTGTATAAGAGACAGgaggagccagtt
gtayctc-3′
TAR2_RACE-5E_mG2b_rev
(SEQ ID NO: 108)
5′-GTCTCGTGGGCTCGGAGATGTGTATAAGAGACAGgaaccagttgta
tctccacac-3′
TAR2_RACE_5E_G3_rev
(SEQ ID NO: 109)
5′-GTCTCGTGGGCTCGGAGATGTGTATAAGAGACAGgatccagatgtg
tcactgc-3′
LIGHT CHAIN oligos (for both kappa and lambda
classes):
TAR2_RACE-5E_mKappaCL_rev
(SEQ ID NO: 110)
5′-GTCTCGTGGGCTCGGAGATGTGTATAAGAGACAGgtgggaagatgg
atacagttg-3′
TAR2_RACE-5E_mLambda1/4_rev, Tm 65° C.
(SEQ ID NO: 111)
5′-GTCTCGTGGGCTCGGAGATGTGTATAAGAGACAGgagctcttcaga
ggaaggtg-3′
TAR2_RACE-5E_mLambda2/3_rev
(SEQ ID NO: 112)
5′-GTCTCGTGGGCTCGGAGATGTGTATAAGAGACAGctcagrggaagg
tggaaac-3′

A common forward oligo was used:

TAR1_XSCTnTag_V3.17
(SEQ ID NO: 113)
5′-Tcgtcggcagcgtcagatgtgtataagagacagtttttttttttt
ttttt-3′

Cloning into commercial plasmid (and transformation) was performed using ZeroBlunt TOPO PCR Kits according to manufacturer's instructions (Invitrogen). Colonies were then picked, and plasmids extracted using the Qiaprep Miniprep Kit (Qiagen) and Sanger Sequences was performed.
100ng of plasmid was used and the following oligos:

M13 Forward
(SEQ ID NO: 114)
5′-GTAAAACGACGGCCAG-3′
M13 Reverse
(SEQ ID NO: 115)
5′-CAGGAAACAGCTATGAC-3′

QIAquick Gel Extraction Kit and MinElute PCR Purification Kit (Qiagen) were used for the DNA purification steps.

### Analysis of Heavy Chain Sequencing

Upon analysis, the sequences obtained for every tested clone share the following regions organization;
polyT sequence
5′ UTR (UnTranslated Region)+leader
Full variable heavy (VH) domain
Short region of CHI domain
NGS adapters (5′/3′) [needed only if DNA submitted to NGS sequencing]
   Using a sequence analysis software (DNASTAR LaserGene 12), everything was discarded but the antibody genes (VH and CHI). Upon alignment, all sequences show complete homology. Table 3 lists the features of the sequence obtained.

TABLE 3
VH Sequence

| | | |
|---|---|---|
| VH Sequence | VH Nucleic | GAGGTTCAGCTCCAGCAGTCTGGGACTGTGCTG |
| Analysis | Acid (SEQ ID | GCAAGGCCTGGGGCTTCCGTGAAGATGTCCTGC |
| | NO: 93) | AAGGCTTCTGGCTACAGCTTTACCAGCTACTGG |
| | | ATGCACTGGGTAAAACAGAGGCCTGGACAGGG |
| | | TCTAGAATGGATTGGTGCTATTTATCCTGGAAA |
| | | TAGTGATACTAGCTACAACCAGAAGTTCAAGG |
| | | GCAAGGCCAAACTGACTGCAGTCACATCCGCC |
| | | AGCACTGCCTACATGGAGCTCAGCAACCTGAC |
| | | GAATGAGGACTCTGCGGTCTATTCCTGTACATT |
| | | ACTACGTTTCCTCGATGCTTACTGGGGCCAAGG |
| | | GACTCTGGTCACTGTCTCTGCAG |
| | VH Amino | EVQLQQSGTVLARPGASVKMSCKASGYSFTSYW |
| | Acid (SEQ ID | MHWVKQRPGQGLEWIGAIYPGNSDTSYNQKFKG |
| | NO: 94) | KAKLTAVTSASTAYMELSNLTNEDSAVYSCTLLR |
| | | FLDAYWGQGTLVTVSA |

### Analysis of Light Chain Sequencing

TABLE 4
summarizes the data for the productive light chain.

| | | |
|---|---|---|
| VL Sequence | VL Nucleic | CAAATTGTTCTCTCCCAGTCTCCAGCAATCCT |
| Analysis | Acid (SEQ ID | GTCTGCATCTCCAGGGGAGAAGGTCACAATG |
| | NO: 95) | ACTTGCAGGGCCAGCTCAAGTGTAAGTTACAT |
| | | GCACTGGTACCAGCAGAAGCCAGGATCCTCCC |
| | | CCAAACCCTGGATTTATGCCACATCCAACCTG |
| | | GCTTCTGGAGTCCCTGCTCGCTTCAGTGGCAG |
| | | TGGGTCTGGGACCTCTTACTCTCTCACAATCA |
| | | GCAGAGTGGAGGCTGAAGATGCTGCCACTTAT |
| | | TACTGCCAGCAGTGGAGTAGTAACCCACCCAC |
| | | GTTCGGAGGGGGGTCCAAGCTGGAAATAAAA |
| | VL Amino | QIVLSQSPAILSASPGEKVTMTCRASSSVSYMHW |
| | Acid (SEQ ID | YQQKPGSSPKPWIYATSNLASGVPARFSGSGSGT |
| | NO: 96) | SYSLTISRVEAEDAATYYCQQWSSNPPTFGGGS |
| | | KLEIK |
| | CL | Kappa class |

Two aberrant transcripts were also identified (aberrant transcripts k138 and k142) as described in Cablilly and Riggs, Gene. 1985; 40(1):157-61. The aberrant chains are likely not contributing to any binding activity.

### CONCLUSIONS

The VH and VL antibody genes of hybridoma clone FHUSPA2/10 were successfully amplified and sequenced.
Isotype of FHUSPA2/10 antibody was confirmed as IgG2a.

### Example 2: FHUSPA2/10 Epitope Mapping by HDX-MS

Materials: Deuterium oxide (99.9% D atoms), sodium deuteroxide, deuterium chloride, acetonitrile and Glu-fibrinogen peptide (GFP) were all purchased from Sigma-Aldrich and used without further purification. Poroszyme immobilised pepsin column was purchased from Thermo-Fisher.

### Methods

### Sample Preparation for HDX-MS Analyses.

1. The antibody/antigen complex was formed by adding 378 pmoles (pico moles) of UspA2 (SEQ ID NO: 53) monomer to the FHUSPA2/10 antibody using a molar ratio UspA2 monomer/mAb of 1:0.33 (or expressed as 3:1 UspA2: mAB) or 1:1 and incubated for 30 min at 25° C.
2. The labelling was initiated by adding deuterated PBS buffer (pH 7.3), reaching a deuterium excess of 87.3%, at 25° C. Over the time course of the experiment (ranging from 30 secs to 24 hours), 30 μL of the sample were removed and quenched with the same volume of an ice-cold quenching buffer (7M urea, 400 mM GuCl, 800 mM TCEP, 0.1% F.A., pH 2.4) to dissociate the antibody/antigen complex and to lower the pH to 2.4. The quenched aliquots were immediately frozen in liquid nitrogen and stored at −80° C. for less than 24 h.

A control experiment without antibody was prepared using the same conditions previously described (PBS was used instead of the antibody preparation). Labelled samples were immediately flash frozen in liquid nitrogen and stored at −80° C. for less than 24 h.

### Local HDX-MS Analyses

Labelled samples were thawed rapidly to 0° C. and injected into a Waters nanoACQUITY UPLC with HDX Technology. The injector, switching valve, columns, solvents and all associated tubings were at 0° C. to limit back-exchange. For local HDX-MS, protein samples were on-line digested for 2.5 min at 20° C. with a flow rate of 200 μL/min using a Poroszyme Immobilized Pepsin Cartridge (2.1 mm×20 mm, Thermo-Fisher) equilibrated with 100% buffer A (2% acetonitrile, 0.1% formic acid in water). The generated peptides were immediately trapped, concentrated and desalted using a VanGuard BEH Pre-column (1.7 μm, 2.1×5 mm, Waters). The 2.5 min digestion and desalting step allows deuterons located at fast exchanging sites (i.e. side chains and amino/carboxy terminus) to be replaced with hydrogens. Peptides were then separated on an ACQUITY UPLC BEH C18 reverse phase column (1.7 m, 1.0×100 mm, Waters) with a linear gradient from 10 to 40% buffer B (2% water, 0.1% formic acid in acetonitrile) over 6.8 min at 40 μL/min.
Mass spectra acquisition: Mass spectra for the epitope mapping experiments with mAb FHUSPA2/10 were acquired in resolution mode (m/z 300-2000) on a Waters SynaptG2 mass spectrometer equipped with a standard ESI source. The mass spectrometer SynaptG2 was calibrated before each analysis with a Caesium iodide solution (2 mg/mL in 50% isopropanol) infused through the reference probe of the ESI source. Mass accuracy was ensured by continuously infusing a GFP solution (600 fmol/μL in 50% (v/v) acetonitrile, 0.1% (v/v) formic acid) through the reference probe of the ESI source. The identity of each peptide was confirmed by MS^{E}analyses. MS^{E} was directly performed by a succession of low (6 V) and high collision (25 V) energies in the transfer region of the mass spectrometer. All fragmentations were performed using argon as collision gas. Data were processed using Protein Lynx Global Server 2.5 (Waters) and each fragmentation spectrum was manually inspected to confirm the assignment. The DynamX software (Waters) was used to select the peptides considered for the analysis and to extract the centroid mass of each of them, and for each charge state, as a function of the labelling time. Only the peptic peptides present in at least four over five repeated digestions of the unlabelled proteins were considered for the analysis.
Synapt G2 settings:

### Source: ES+

### Capillary: 3000 V

### Sample Cone: 25 V

### Extraction Cone: 4 V

### Source Temperature: 80° C.

### Cone gas: 20 L/h

### Results

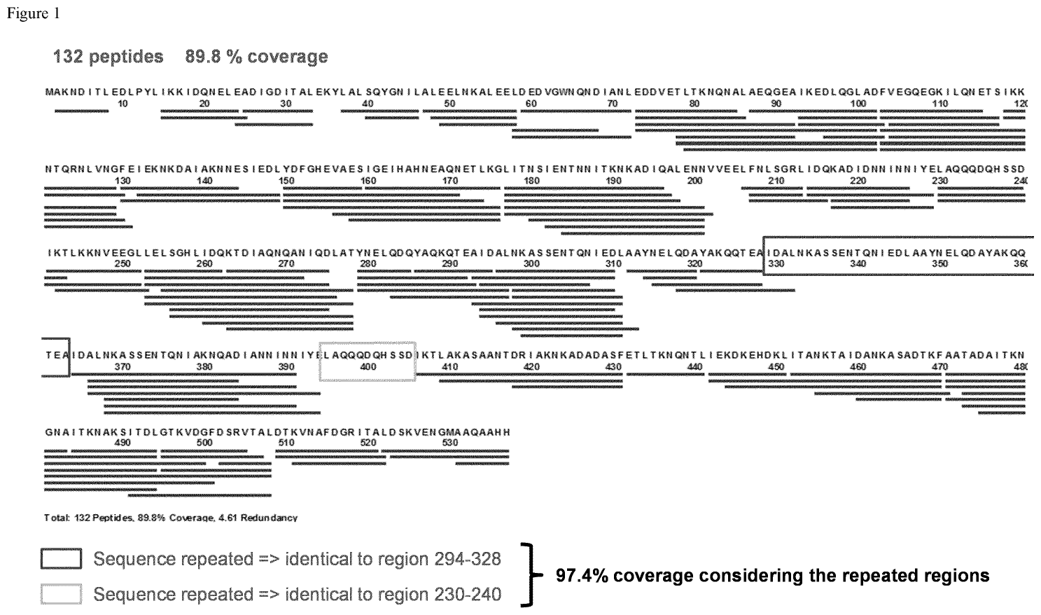

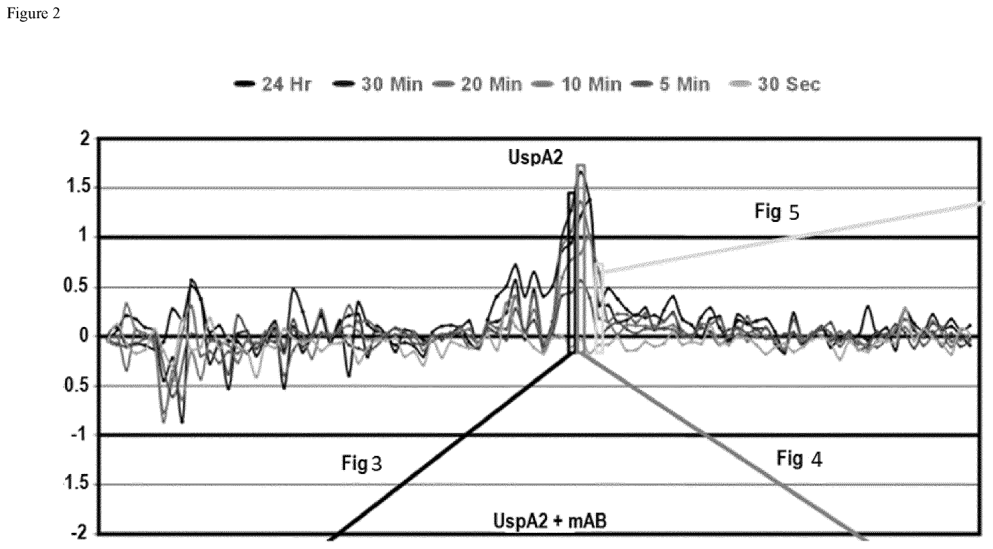

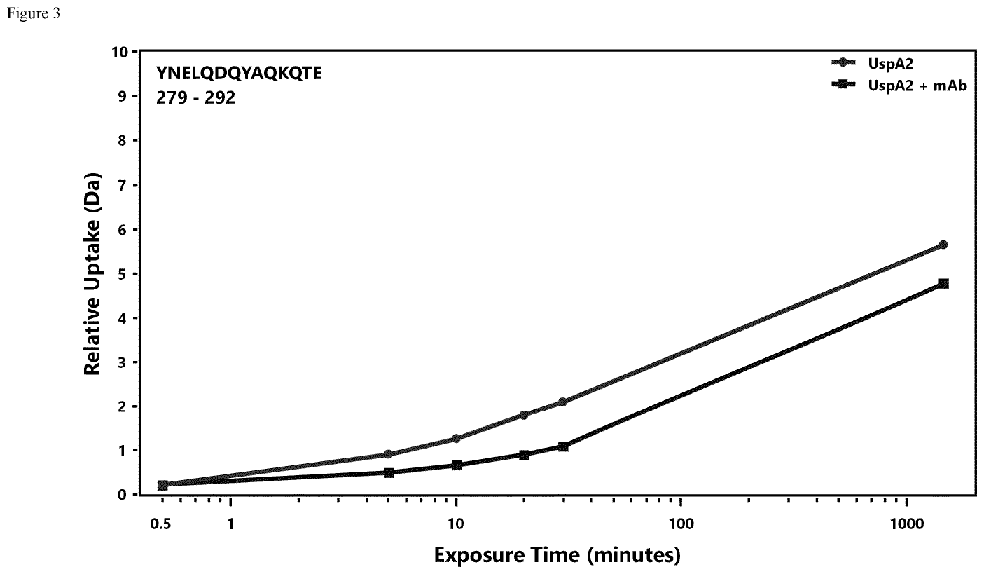

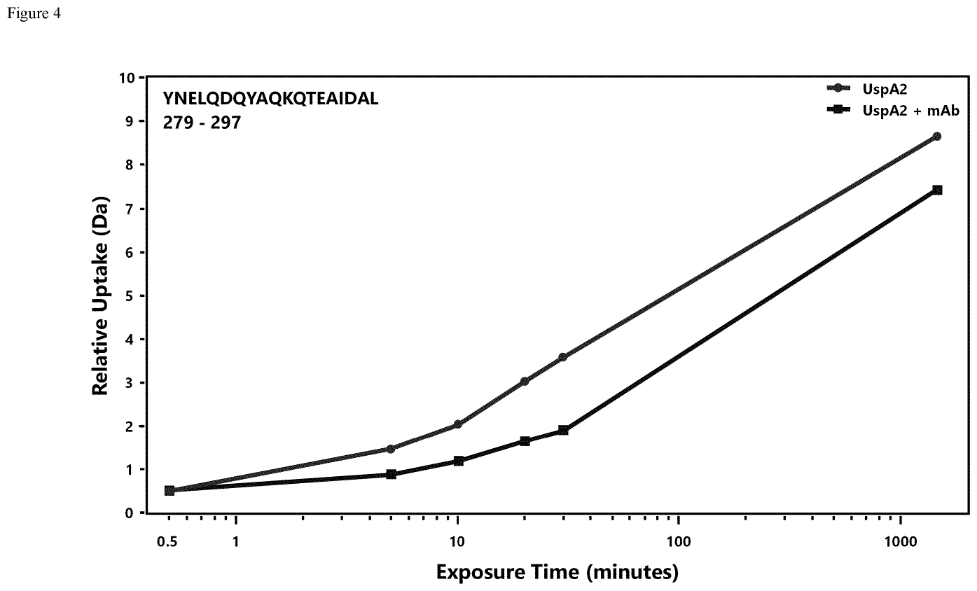

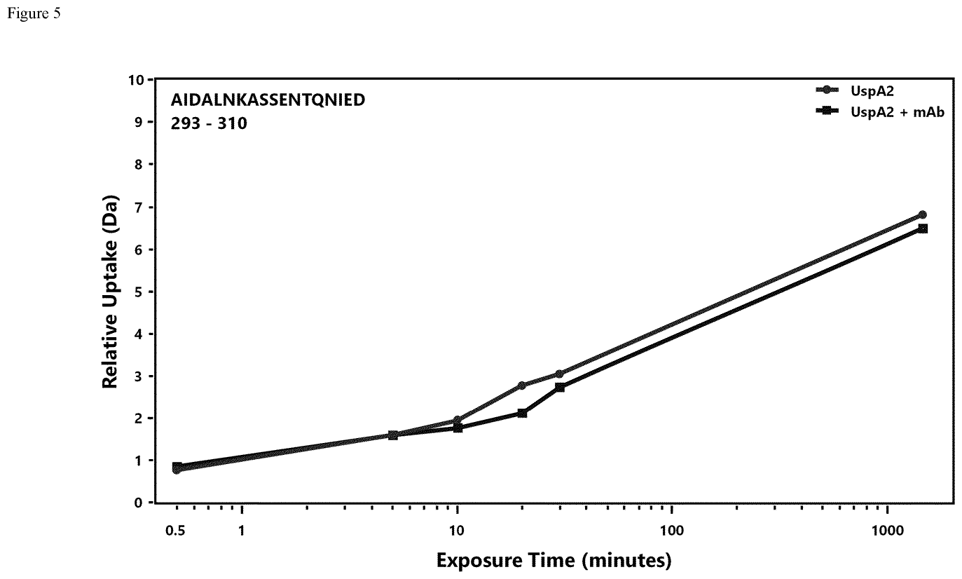

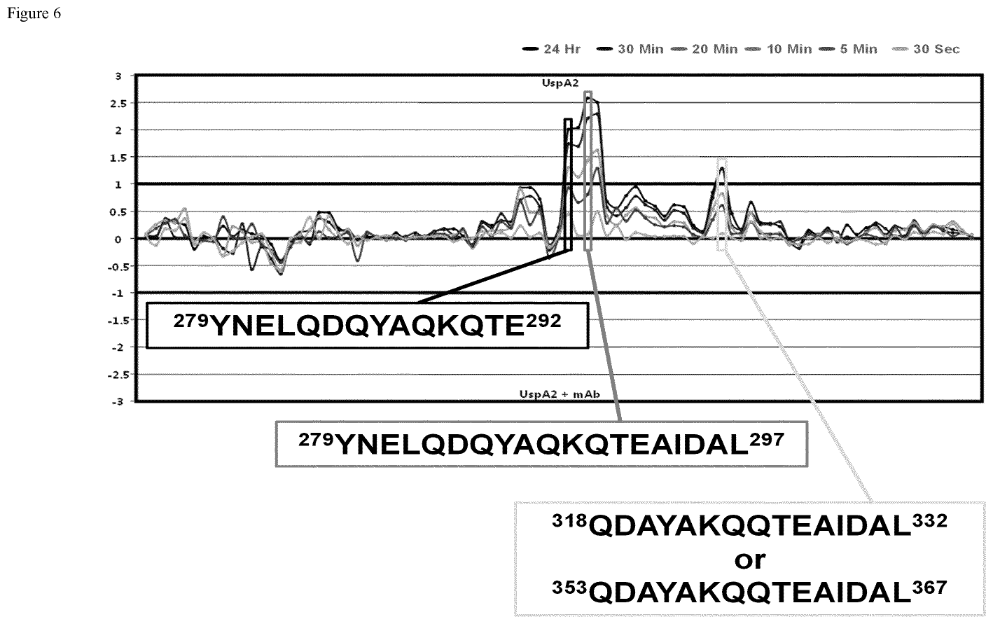

The epitope mapping of the UspA2 protein construct MC-009 (SEQ ID NO: 75) protein with the FHUSPA2/10 antibody was performed using the Waters nanoACQUITY UPLC with HDX Technology and DynamX software.
132 pepsin peptides, corresponding to 89.8% of the UspA2 sequence were considered for this analysis (FIG. 1). In the protein are present repeated regions with the same aminoacidic sequence, in particular region 294-328 is identical to region 329-363 and region 230-240 is identical to region 395-405. Considering the repeated regions, the overall sequence coverage is 97.4%.
The deuterium incorporation on these 132 peptides generated from the antigen under its free or mAb-bound form with a molar ratio of 1:0.33 can be visualised in FIG. 2. The difference of deuterium incorporation was considered significant when the averaged value of deuterium incorporation is superior to 1 Da.
Peptides Y279-E292 (FIG. 3) and Y279-L297 (FIG. 4) showed a significant difference in deuterium uptake in presence of the mAb. By considering the incorporation of other peptides in the same region that does not show differences, like peptide A293-D310 (FIG. 5), it is possible to reduce the epitope sequence to peptide 279-292 (YNELQDQYAQKQTE). This sequence is part of a domain repeated three times in the UspA2 (SEQ ID NO: 75) with some little amino acid differences. The differences with the other two repeated regions consist in the substitution of Q with A and the inversion of QKQ into KQQ. These residues are important for the specificity.
Increasing the amount of mAb compared to the protein (protein monomer/antibody molar ratio of 1:1), will result in a binding also in the repeated regions as reported in FIG. 6.

### Example 3: Production of UspA2 pAb

Rabbits were immunized 3 times (day 0, 14 and 35) with recombinant UspA2 of SEQ ID NO: 75 (lot no: BMP115) (523 gg/ml) adjuvanted with Specol or Specol alone. Bleeding was performed at day 42 to assess the immunogenicity and the final bleeding was performed at day 49.
Total IgG was purified from serum (lot no: LIMS20110052) using Nab Protein A Plus Spin Columns (Thermo Fisher Scientific) according to manufacturer's recommendations. IgG purification was followed by SDS-PAGE analysis and IgG was quantified using the Lowery assay (as described in Lowry, O. H., Rosebrough, N. J., Farr, A. L., and Randall, R. J. 1951. Protein measurement with the Folin phenol reagent. J. Biol. Chem. 193:265-275).
The capability of the polyclonal antibody to recognize recombinant UspA2 was assessed by Western Blot analysis. After purification, antibody was stored at −20° C. in PBS at a final concentration of 2.18 mg/ml until required for use.
UspA2 specific IgG was purified by immunoaffinity. The antigen was coupled on mL HiTrap NHS-activated column (GE Healthcare) according to the manufacturer's instructions (ref instructions 71-7006-AW). 7.2 mg of total IgG in 3.3 mL were diluted to 5 mL with PBS and loaded on the antigen-coupled column equilibrated with PBS. The flow through was collected for a second load onto the column and the UspA2 specific antibodies were eluted with 0.1M Glycine pH 2.9. Each fraction was neutralized by adding 1/10 vol of 1M Tris pH 10 in the collecting tube before elution. The purified IgG were then dialysed against PBS and 200 μg of UspA2 specific IgG were obtained.

### Example 4: UspA2 pAb Epitope Mapping

The sample preparation for HDx-MS experiment was performed as reported for the sample preparation of UspA2 mAb epitope mapping with the following modifications. The antigen/pAb complex was formed by adding 15 pmoles of UspA2 monomer to rabbit specific IgG pool using a molar ratio UspA2 monomer/IgGs of ½. Anti-PD total IgG were used as a negative control in the same molar ratio. The labelling was initiated by adding deuterated PBS buffer (pD of 7.3), reaching a deuterium excess of and 82.4% (UspA2—rabbit specific IgG pool and negative control) at 25° C. Mass spectra were acquired in resolution mode (m/z 300-2000) on a Waters SynaptG2-Si mass spectrometer equipped with a standard ESI source. The setting of the instruments were:

### Source: ES+

### Capillary: 3000 V

### Sample Cone: 40V

### Extraction Cone: 5V

### Source Temperature: 80° C.

### Cone gas: 0 L/h

### Results

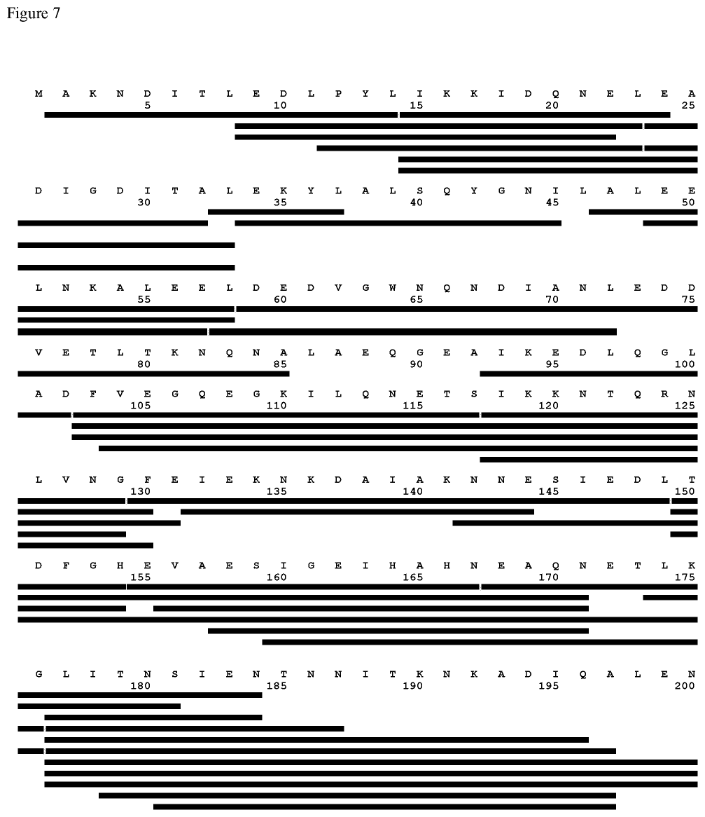

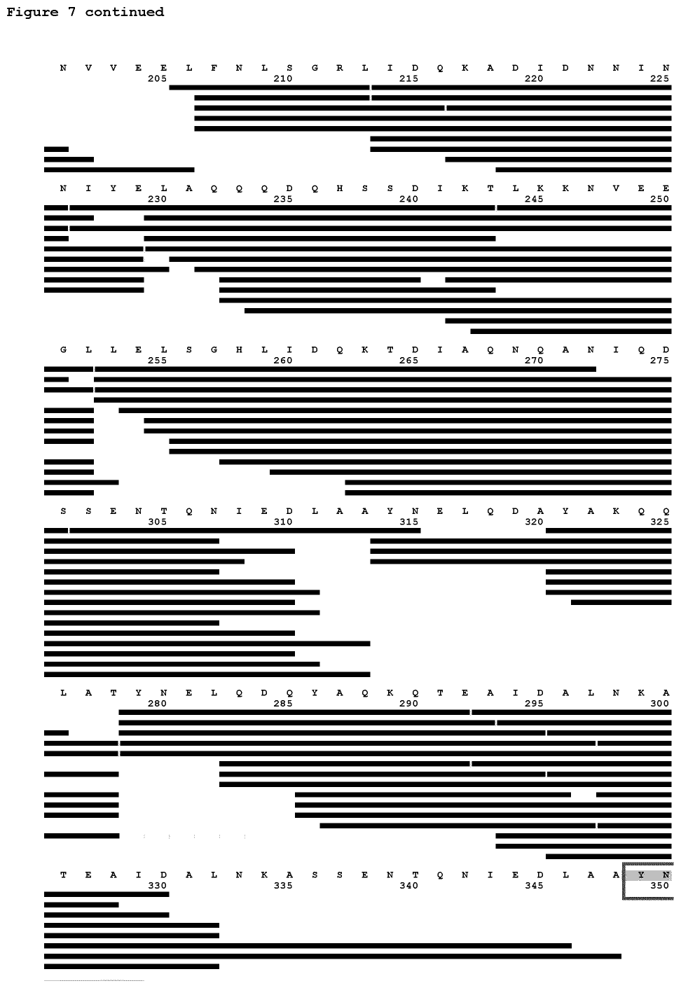

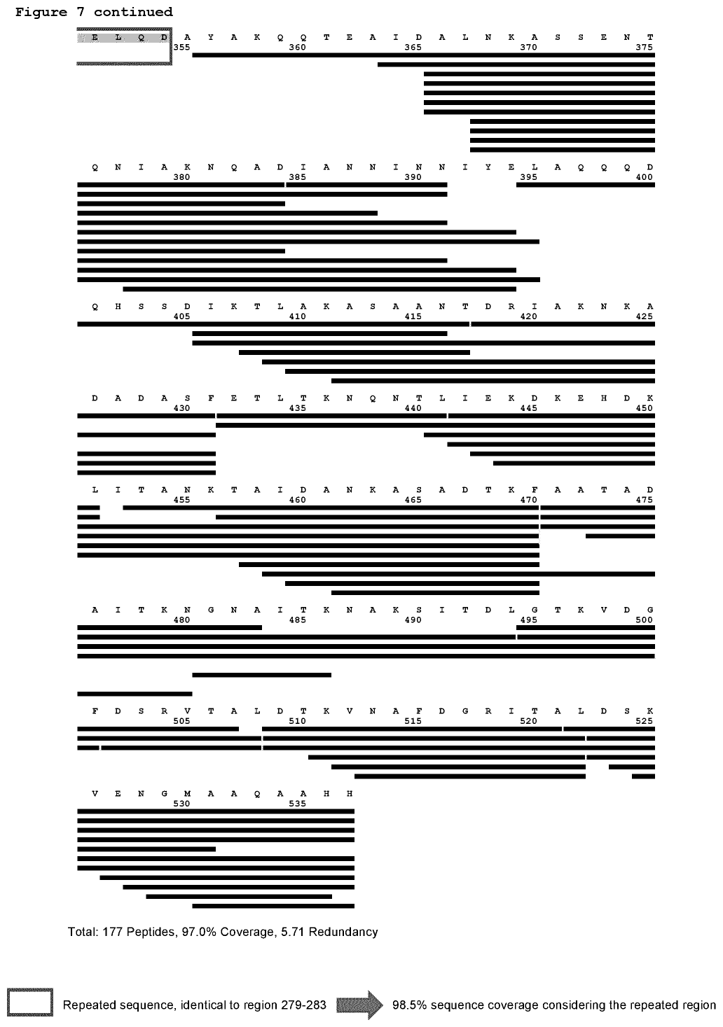

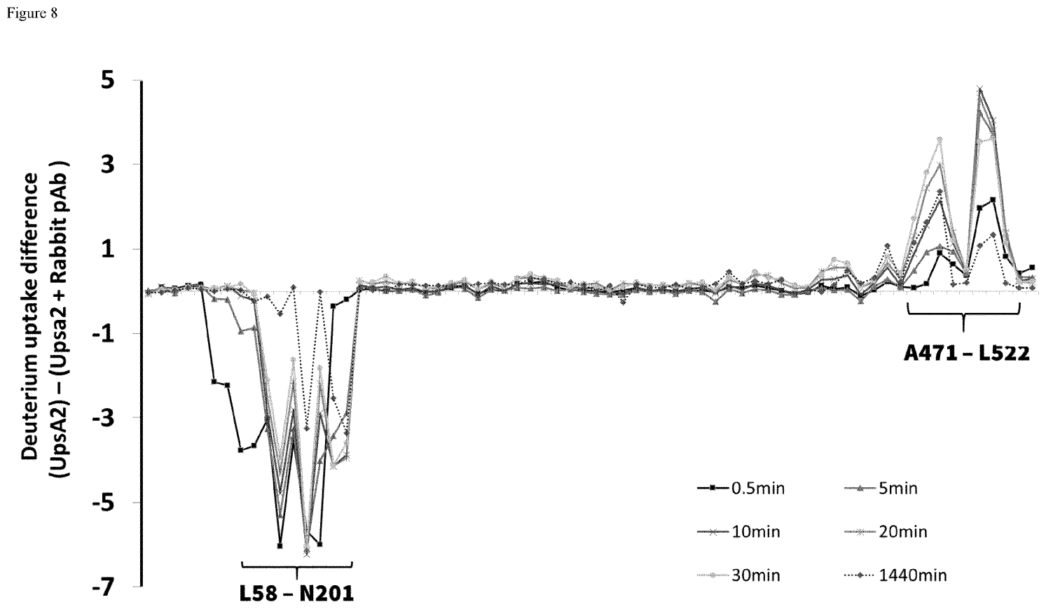

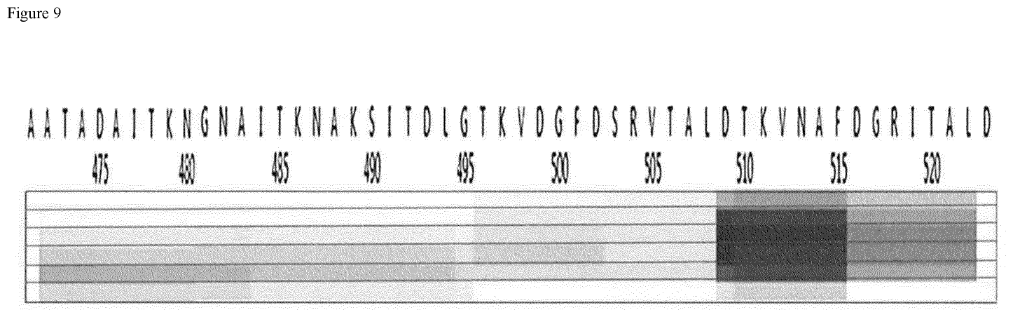

Mapping of UspA2 epitopes recognised by UspA2-specific IgG purified from rabbit serum was performed using the Water nanoACQUITY UPLC with HDX Technology associated with a Synpat G2-Si.
The recombinant UspA2 was digested by pepsin and the resulting peptides were analysed by MS/MS. 177 peptides corresponding to 98.5% of the UspA2 sequence were identified by PLGS (FIG. 7).
Among those 177 peptides, 68 were considered for the analysis of the deuterium incorporation of the antigen alone and the complex antigen/pAb (results shown in FIG. 8 and FIG. 9 (zoomed map for fragment A471-L522).

### Results

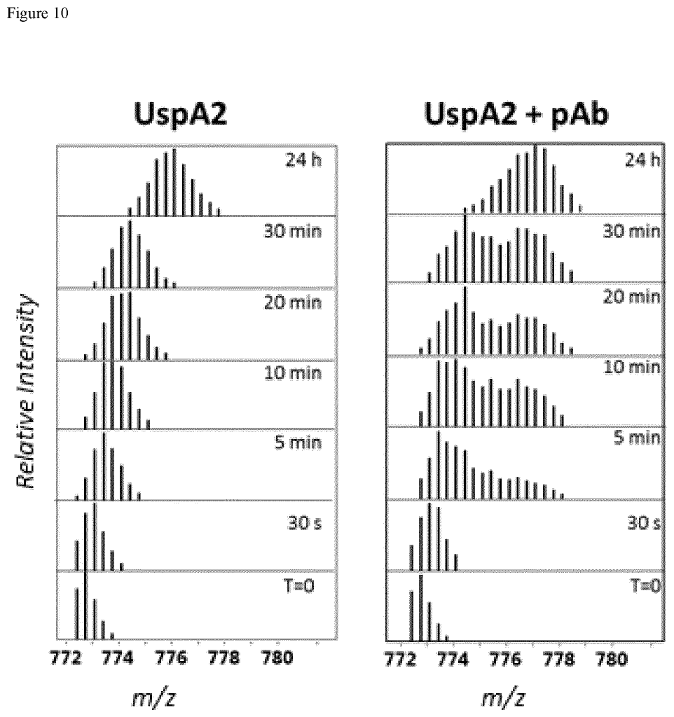

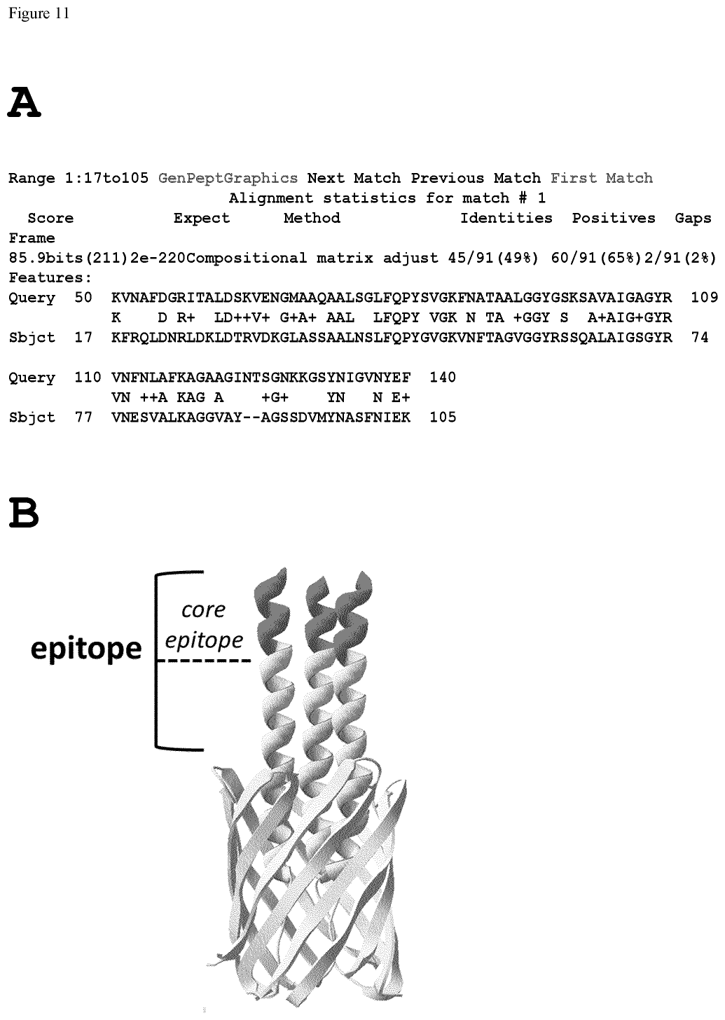

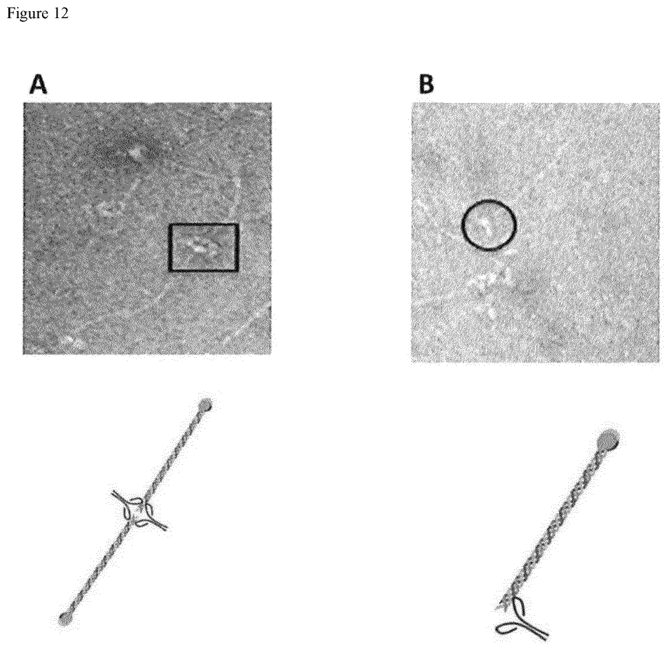

Reduction of deuterium incorporation upon the binding of UspA2-specific IgG to the antigen was observed for 7 overlapping peptides located at the antigen C-terminal region covering the sequence A471-L522, (A471-A483, A471-L494, A471-L508, G495-L508, D502-L508, D509-L522 and F515-522). To better define the sequence, the residues showing the highest difference of deuterium uptake were identified using the function “heat map” of the software DynamX. The difference in deuterium uptake between antigen and antigen-pAb is expressed as a percentage of maximum exchangeable amides for each peptide considered. The function narrows the localization of deuterium incorporation from overlapping labelled peptides. In this specific case, the UspA2 backbone amides presenting the highest level of protection to the deuterium exchange corresponds to those of residues D5090-F515 (DTKVNAF, SEQ ID NO: 116) defining the core of the immune-dominant epitope, while adjacent residues D516-L521 (DGRITAL, SEQ ID NO: 117) also participates to the epitope. Minor effects were observed in the region A472-L508 (ATADAITKNGNAITKNAKSITDLGTKVDGFDSRVTAL, SEQ ID NO: 118).
To visualise the immune-dominant region recognized by the rabbit pAb, a computer model of the UspA2 translocation unit (residues 508-601) was built by threading the UspA2 sequence onto the X-ray coordinates of the Yersinia enterocolitica trimeric autotransporter YadA (PDB code 2LME). The 49% identity of the 501-601 UspA2 ATCC_25238 residues with Y. enterocolitica supports the use of the X-ray structure as template to predict the conformation of the 501-601 region of UspA2. (FIG. 11). The model has been generated with Swiss-PDB-Viewer Software (version 4.1). Two residues have been added at the N-terminal in alpha helix conformation to model the entire immune dominant region recognised by the polyclonal serum. Such addition is justified by the sequence analysis of the protein, which predicts a helical coiled coil conformation for the entire passenger domain and the body of the knowledge on the structures determined for members of the trimeric coiled coil ahesins. The resulting model is shown in FIG. 11 B. The immune-dominant epitope recognised by IgG raised from rabbit immunized with the recombinant UspA2 is part of the UspA2 translation unit, indicating a very proximal location to the bacterial outer membrane.
Interestingly, the binding of the UspA2 antigen with pAb induced an effect on the UspA2 structure in a region spanning L58-N201 (although no information was available for the region E79-193), see FIG. 8. As an example, the spectra of the peptide L177-A197 is shown in FIG. 10. In presence of UapA2 alone or with uncorrelated pAb, the deuterium incorporation kinetics of the peptide produces a gradual increase in average mass (FIG. 10 left panel). This pAb destabilizing effect on UspA2 antigen is not expected to impact the IVRP assay, since the mAb used for antigen immobilization recognizes an epitope present in a region that is not affected by the binding of the pAb
To gain information on the immune-dominance of the rabbit pAb, the complexes antigen/pAb, were also observed by Negative Staining (NS)-TEM. NS-TEM techniques are known in the art.
One binding site of the UspA2 C-terminal region as described above was observed. Each IgG molecule bound either one single molecule of the antigen or, very frequently, two UspA2 copies were positioned in a tail-tail orientation and connected by two IgG copies through their C-terminal epitopes (FIG. 12).

### Example 5: Antibody Cross-Bactericidal Activity—FHUSPA2/10 mAB and pAb

### Bactericidal Assay

Moraxella catarrhalis was cultivated overnight on Petri dish at 37° C.+5% CO₂. Bacteria were transferred in 12 ml HBSS-BSA (Hank's Buffered Salt Solution with Bovine Serum Album) 0.1% buffer in order to get an OD₆₂₀ of 0.650. Serum samples were heated for 45 min at 56° C. to inactivate the endogenous complement. Serial two-fold dilutions of sera in serum bactericidal assay (SBA) buffer (HBSS-BSA 0.1%) were added on a 96-well round bottom microtiter plate (25 μl/well). Subsequently, 50 μl of SBA buffer were added in each well. Then 25 l of Moraxella catarrhalis strains at 4.104 CFU (colony forming unit)/ml were added to the wells containing sera and incubated for 15 min at room temperature. Finally, 25 μl of freshly thawed baby rabbit complement diluted 1/8 in HBSS-BSA 0.1% were added to reach a final volume of 125 l. Plates were incubated for 1 h at 37° C. with orbital shaking (210 rpm). The reaction was stopped by laying the microplate on ice for at least 5 min.

After homogenization, various dilutions of the suspension (a mixture of bacteria, serum, complement and buffer, at a volume of 125 μl as discussed in the previous paragraph) were added onto chocolate agar plates and incubated for 24 hours at 37° C. with 5% CO₂ and Moraxella catarrhalis colonies were counted.

Eight wells without serum sample were used as bacterial controls to determine the number of Moraxella catarrhalis colonies per well. The mean number of CFU of the control wells was determined and used for the calculation of the killing activity for each serum sample. The bactericidal titers were expressed as the reciprocal dilution of serum inducing 50% of killing (mid-point titer).

### Results

The anti-UspA2 monoclonal antibody FHUSPA2/10 was tested in the bactericidal assay described here above against 8 different Moraxella catarrhalis strains isolated in various countries (UK, Denmark, Netherlands), that are representative of UspA2 variability.
As shown below (table 5) the anti-UspA2 monoclonal antibody FHUSPA2/10 was able to induce a cross-bactericidal killing of Moraxella catarrhalis, whatever the percentage of homology of the UspA2 expressed by the tested strain. Moreover, bactericidal activity was also shown against strains expressing the chimeric protein UspA2H. As expected, no bactericidal activity was measured against an UspA1/UspA2 double knock-out mutant.

TABLE 5
Cross-bactericidal activity of the anti-UspA2 monoclonal antibody FHUSPA2/10

| | | | | Identity %* | |
|---|---|---|---|---|---|
| | | | | versus the | FHUSPA2/10 |
| | | | | vaccine | monoclonal |
| | | | | sequence | antibody |
| | | | | ATCC 25238 | bactericidal |
| | | | UspA gene | (SEQ ID NO: | activity |
| M. catarrhalis strains | | Source | present | 1) | (mid-point titer) |
| ATCC | 25238 | Not known | UspA1/UspA2 | 45.2/100 | 1881667 |
| | 43617 | Sputum from a | UspA1 | 40.1 | <100 |
| | | Chronic | | | |
| | | Bronchitis and | | | |
| | | COPD Patient | | | |
| | 296 | Sputum from a | UspA1/UspA2H | 40.8/64.4 | 113906 |
| | | COPD patient | | | |
| UK isolates | 415 | Sputum from a | UspA1/UspA2H | 37.1/64.6 | 256441 |
| (AERIS | | COPD patient | | | |
| study) | 268 | Sputum from a | UspA1/UspA2H | 35.8/57.3 | >384000 |
| | | COPD patient | | | |
| | 240 | Sputum from a | UspA2H | 68.4 | >25600 |
| | | COPD patient | | | |
| | 193 | Sputum from a | UspA1/UspA2H | 47.1/72.5 | >640000 |
| | | COPD patient | | | |
| Dutch isolate | F10 | Sputum | UspA1/UspA2 | 42.9/61.1 | 527667 |
| | | from a patient | | | |
| | | with a lower | | | |
| | | respiratory tract | | | |
| | | infection | | | |
| Denmark | BBH18 | Sputum | UspA1/UspA2 | 42.9/57.3 | 385784 |
| isolate | | from a COPD | | | |
| | | patient with | | | |
| | | exacerbation | | | |
| | BBH18 | Mutated form of | No UspA gene | / | <100 |
| | UspA1 & | BBH18 | | | |
| | UspA2 | | | | |
| | double | | | | |
| | knock out | | | | |

*determined using the software GapL/ClustalX

The assessment of functional activity, as measured by the rabbit Serum Bactericidal Assay (rSBA) of the polyclonal antibodies (pAbs) against UspA2 is reported below.
High bactericidal titers (see table 6 below) are observed with the rabbit polyclonal anti-UspA2 antibody against both the homologous (with respect to the UspA2 vaccine sequence) ATCC 25238 M. catarrhalis strain and the heterologous F10 strain (expressing an UspA2 having 53% of sequence homology with the vaccine antigen).

TABLE 6
Bactericidal titers of the pAb anti-UspA2 (reciprocal dilution of
antibodies inducing 50% of killing (GMT))

| Mcat strains | ATCC 25238 Mcat strain | heterologous F10 strain |
|---|---|---|
| pAb bactericidal | 38769 | 22699 |
| titer | | |

The cross-bactericidal activity of the rabbit anit-UspA2 pAb was demonstrated against further strains of M. catarrhalis (such as those isolated in the AERIS study) as shown in Table 7 below.

TABLE 7

| BBH18 | | | | | | | |
|---|---|---|---|---|---|---|---|
| UspA1 | | | | | | | |
| KO/ | | | | | | | |
| UspA2 | BBH18 | AERIS | AERIS | | AERIS | | |
| KO | WT | 296 | 415 | AERIS 268 | 240 | AERIS 193 | AERIS 443 |

% IDENTITY VERSUS VACCINE SEQUENCE

| | | | | | | | | |
|---|---|---|---|---|---|---|---|---|
| UspA1 | / | 39.7 | 40.82 | 37 | 35.8 | / | 43.1 | 46.4 |
| UspA2 | / | 2H \| 63.3 | 2H \| 64.4 | 2H \| 64.6 | 2H \| 57.3 | 2H \| 68.4 | 2H \|72.5 | 2 \| 82.2 |

BACTERICIDAL TITERS

| | | | | | | | | |
|---|---|---|---|---|---|---|---|---|
| Anti- | 825- | 2849-4257 | 6601- | 3233- | 4155 | >12800 | >12800 | <100 |
| Whole | 2591 | | 11291 | 5016 | | | | |
| Cells | | | | | | | | |
| Rabbit | <100 | 19823 | 6820 | 28484 | 8802-10239 | >12800 | 651-1753 | 1204-1980 |
| pAb anti- | | | | | | | | |
| UspA2 | | | | | | | | |

### Example 6—IVRP Assay

### Protocol

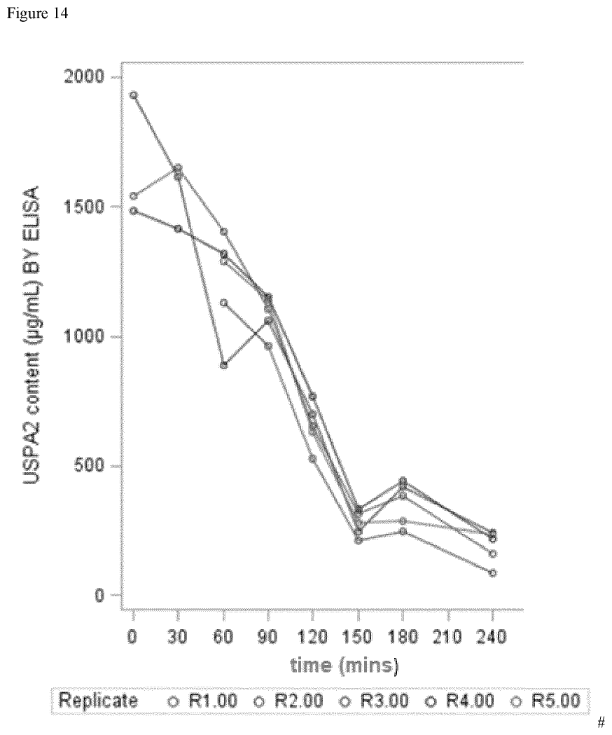

Microtiter 96-well plates (MAXISORP™, Nunc Thermo Scientific) were coated 30 min at 37° C. with 100 μl per well of FH USPA2/10 purified mAb at 3.184 μg/ml diluted in Phosphate Buffer Saline (PBS). Having washed the plates four times with NaCl 0.9% Tween 20 0.05%, reference, internal control and samples were added at 0.2 μg/ml in the first well then diluted from line A to H according a 3—fold serial dilution in PBS Tween 20 0.05%. Reference and internal control were included in each test.
The plates were incubated for 75 min at 25° C. After washing, anti-UspA2 rabbit pAb was added at a dilution 1/8000 (100 μl per well) for 30 min at 25° C. Plates were washed as above and peroxidase conjugated goat anti-rabbit was added at a dilution 1/4000 (100 l per well) for 30 min at 25° C. After washing, the solution of revelation [4 mg of OPDA and 5 l of H₂O₂ in 10 ml of citrate 0.1M pH 4.5 buffer] was added to each well (100 μl per well) for 15 min in darkness. The reaction was stopped by addition of 50 μl of H2SO4 IN and the optical density (OD) is read at 490 nm (620 nm for the reference filter).
Relative potency (%) of each sample can be determined by full logistic curve parallelism method using SoftMax Pro software.
FIG. 14 shows the results of an experiment where a test sample comprising recombinant UspA2 (of SEQ ID NO: 75) was thermally stressed at 45° C. for up to 4 hours. As can be seen, UspA2 content as detected by the IVRP ELISA assay described above is significantly reduced by 3 hours post-exposure (to level of 8.5% compared to an unstressed sample).

### Example 7: Use of the IVRP Assay for Assessing Antigenicity of Stressed UspA2 Drug-Substance

The UspA2 drug substance (DS) was batch produced at final scale. The compoisiton of the lot (assigned the reference number: EUSPGPA018) was as follows; 1.5 mg/ml UspA2 (SEQ ID NO: 75), 10 mM P04 (KH2/K2H), Arginine, pH 6.5.

### Heat

As described in Example 6, the UspA2 antigen is sensitive to heat. To confirm this observation using the final scaled up drug-substance, the UspA2 DS (lot number: EUSPGPA018) was thermally stressed at 45° C. for up to 3 hours. As shown in Table 8 below a decrease in antigenic activity was observed using the IVRP ELISA assay of the invention.

### Oxidation

The impact of oxidation on the UspA2 DS (lot number: EUSPGPA018) was assessed using the accelerated oxidative test (AOT). The UspA2 DS was exposed to light in a chamber for 19h, 600 W/m² (corresponding to a light exposure 20× stronger compared to what it is in the DS production area). Accelerated oxidative test (AOT) did not however affect significantly the UspA2 protein and no impact was observed on IVRP UspA2 by ELISA (see table 8 below)

### Deamidation

UspA2 protein is known to be sensitive to asparagine deamidation, which is promoted at higher pH. However, incubation at pH 8.0 (for 7 days at 25° C.) did not impact IVRP of UspA2 by ELISA (see table 8 below).
In a further study UspA2 antigenic activity was also not affected by incubation at pH 8.0 or 9.0, whether incubated at 25 or 37° C. (see table 9 below). Deamidation was shown to take place specifically at Asn528, wherein deamidation increases readily upon incubation at higher pH, to reach nearly 100% at pH 9.0 and +37° C. (data not shown). However, and unexpectedly, the other asparagine residues (e.g. Asn128, Asn180 and Asn480) do not undergo deamidation in the same conditions: the levels of deamidation of Asn128, Asn180 and Asn480 remain below 5% even in the harsher condition (pH 9.0 for 7 days+37° C.).
This means that deamidation of Asn528 does not impact UspA2 antigen activity, which is aligned with structural information (i.e. as Asn528 is located at the very end C-terminal region of the protein, which is away from the functional epitopes).

### Chemotrypsin

Chymotrypsin treatment was performed to clip the UspA2 antigen.
Method: UspA2 DS was incubated at +37° C. with 0.02U of chymotrypsin per 100 μl of UspA2 for 15 min (target: 5% degradation), 30 min (target: 10% degradation) and 120 min (target: 20% degradation). After 120 min, the reaction was stopped by addition of 0.1% v/v SDS, aliquoted and frozen at −70° C. for ELISA analysis.
Although 100% of the protein is clipped after 120 min of incubation with chymotryspin, it has minimal impact on the antigenic activity (90% recovery). This may be explained by the location of the cleavage site: UspA2 is clipped at its N-terminus (e.g. Tyr12, Tyr35 and Tyr41), which corresponds to the globular head in the tridimensional structure, located away from the functional epitopes.

TABLE 8
UspA2 IVRP Assay

| Sample | Relative Potency (%) |
|---|---|
| EUSPGPA018 Control (Untreated) | 106 |
| EUSPGPA018 75 mins at 45° C. | 48 |
| EUSPGPA018 180 mins at 45° C. | 12 |
| EUSPGPA018 AOT with light | 118 |
| EUSPGPA018 AOT without light | 114 |
| EUSPGPA018 pH 8 for 7 days at 25° C. | 104 |
| EUSPGPA018 Chymotrypsin (120 mins) | 90 |

TABLE 9
Deamidation Follow up Study

| | Relative |
|---|---|
| Sample | Potency (%) |
| EUSPGPA018 Control (Untreated) | 112 |
| EUSPGPA018 pH 8 7 days at 25° C. | 114 |
| EUSPGPA018 pH 8 7 days at 37° C. | 114 |
| EUSPGPA018 pH 9 7 days at 25° C. | 98 |
| EUSPGPA018 pH 9 7 days at 37° C. | 96 |

Sequence Listing

| Strain | UspA2 sequences |
|---|---|
| ATCC25238 | MKTMKLLPLKIAVTSAMIIGLGAASTANAQAKNDITLEDLPYLIKKIDQNELEADI |
| (SEQ ID | GDITALEKYLALSQYGNILALEELNKALEELDEDVGWNQNDIANLEDDVETLTKN |
| NO: 1) | QNALAEQGEAIKEDLQGLADFVEGQEGKILQNETSIKKNTQRNLVNGFEIEKNKD |
| | AIAKNNESIEDLYDFGHEVAESIGEIHAHNEAQNETLKGLITNSIENTNNITKNKAD |
| | IQALENNVVEELFNLSGRLIDQKADIDNNINNIYELAQQQDQHSSDIKTLKKNVEE |
| | GLLELSGHLIDQKTDIAQNQANIQDLATYNELQDQYAQKQTEAIDALNKASSENT |
| | QNIEDLAAYNELQDAYAKQQTEAIDALNKASSENTQNIEDLAAYNELQDAYAKQ |
| | QTEAIDALNKASSENTQNIAKNQADIANNINNIYELAQQQDQHSSDIKTLAKASAA |
| | NTDRIAKNKADADASFETLTKNQNTLIEKDKEHDKLITANKTAIDANKASADTKF |
| | AATADAITKNGNAITKNAKSITDLGTKVDGFDSRVTALDTKVNAFDGRITALDSK |
| | VENGMAAQAALSGLFQPYSVGKFNATAALGGYGSKSAVAIGAGYRVNPNLAFK |
| | AGAAINTSGNKKGSYNIGVNYEF (630 aa) |
| American | MKTMKLLPLKIAVTSAMIIGLGAASTANAQSRDRSLEDIQDSISKLVQDDINTLKQ |
| 2933 | DQQKMNKYLLLNQLANTLITDELNNNVIKNTNSIEALGDEIGWLENDIADLEEGV |
| (SEQ ID | EELTKNQNTLIEKDEEHDRLIAQNQADIQTLENNVVEELFNLSGRLIDQEADIAKN |
| NO: 2) | NASIEELYDFDNEVAERIGEIHAYTEEVNKTLENLITNSVKNTDNIDKNKADIDNNI |
| | NHIYELAQQQDQHSSDIKTLKNNVEEGLLELSGHLIDQKADLTKDIKALESNVEEG |
| | LLDLSGRLLDQKADLTKDIKALESNVEEGLLDLSGRLLDQKADIAQNQTDIQDLA |
| | AYNELQDQYAQKQTEAIDALNKASSENTQNIEDLAAYNELQDAYAKQQTEAIDA |
| | LNKASSENTQNIAKNQADIANNINNIYELAQQQDQHSSDIKTLAKASAANTNRIAT |
| | AELGIAENKKDAQIAKAQANANKTAIDENKASADTKFAATADAITKNGNAITKN |
| | AKSITDLGTKVDGFDGRVTALDTKVNAFDGRITALDSKVENGMAAQAALSGLFQ |
| | PYSVGKFNATAALGGYGSKSAVAIGAGYRVNPNLAFKAGAAINTSGNKKGSYNI |
| | GVNYEF (613 aa) |
| American | MKTMKLLPLKIAVTSALIIGLGAASTANAQQQLQTETFLPNFLSNDNYDLTDPFYH |
| 2912 | NMILGDTALLDKQDGSQPQLKFYSNDKDSVPDSLLFSKLLHEQQLNGFKKGDTIIP |
| (SEQ ID | LDKDGKPVYQVDYKLDGKGKKQKRRQVYSVTTKTATDDDVNSAYSRGILGKVD |
| NO: 3) | DLDDEMNFLNHDITSLYDVTANQQDAIKDLKKGVKGLNKELKELDKEVGVLSRD |
| | IGSLNDDVAQNNESIEDLYDFSQEVADSIGEIHAHNKAQNETLQDLITNSVENTNNI |
| | TKNKADIQALENNVVEELFNLSGRLIDQKADLTKDIKTLESNVEEGLLELSGHLID |
| | QKADIAKNQADIAQNQANIQDLAAYNELQDAYAKQQTEAIDALNKASSENTQNI |
| | EDLAAYNELQDAYAKQQTEAIDALNKASSENTQNIAKNQADIANNINNIYELAQQ |
| | QDQHSSDIKTLAKASAANTDRIAKNKADADASFETLTKNQNTLIEKDKEHDKLIT |
| | ANKTAIDENKASADTKFAATADAITKNGNAITKNAKSITDLGTKVDGFDSRVTAL |
| | DTKVNAFDGRITALDSKVENGMAAQAALSGLFQPYSVGKFNATAALGGYGSKSA |
| | VAIGAGYRVNPNLAFKAGAAINTSGNKKGSYNIGVNYEF (644 aa) |
| American | MKTMKLLPLKIAVTSALIVGLGAASTANAQLVERFFPNIFLDKPLAKQHYHNVVV |
| 2908 | GDTSIVSDLQSNSDQLKFYSDDEGLVPDSLLFNKMLHEQLLNGFKEGDTIIPLDEN |
| (SEQ ID | GKPVYKVDYKLDGKEPRKVYSVTTKIATAEDVATSSYANGIQKDIDDLYDFDHQ |
| NO: 4) | VTERLTQHGKTIYRNGERILANEESVQYLNKEVQNNIEHIYELAQQQDQHSSDIKT |
| | LESNVEKGLLELSGHLIDQKADLTKDIKTLESNVEEGLLDLSGRLIDQKADLTKDI |
| | KTLESNVEEGLLDLSGRLIDQKADIAQNQANIQDLAAYNELQDQYAQKQTEAIDA |
| | LNKASSENTQNIEDLAAYNELQDAYAKQQTEAIDALNKASSENTQNIAKNQADIA |
| | NNINNIYELAQQQDQHSSDIKTLAKASAANTNRIATAELGIAENKKDAQIAKAQA |
| | NANKTAIDENKASADTKFAATADAITKNGNAITKNAKSITDLGTKVDGFDSRVTA |
| | LDTKVNAFDGRITALDSKVENGMAAQAALSGLFQPYSVGKFNATAALGGYGSKS |
| | AVAIGAGYRVNPNLAFKAGAAINTSGNKKGSYNIGVNYEF (591 aa) |
| Finnish 307 | MKTMKLLPLKIAVTSAMIIGLGAASTANAQQQQQQQQQQQSRTEIFFPNIFFNENH |
| (SEQ ID | DELDDAYHNIILGDTALLDKQDGSQPQLKFYSNDKDSVPDSLLFSKLLHEQQLNG |
| NO: 5) | FKKGDTIIPLDKDGKPVYQVDYKLDGKGKKQKRRQVYSVTTKTATDDDVNSAYS |
| | RGILGKVDDLDDEMNFLNHDITSLYDVTANQQDAIKGLKKGVKGLNKELKELDK |
| | EVGVLSRDIGSLNDDVAQNNESIEDLYDFSQEVADSIGEIHAHNKAQNETLQDLIT |
| | NSVENTNNITKNKADIQALENNVVEELFNLSGRLIDQKADLTKDIKTLESNVEEGL |
| | LELSGHLIDQKADIAKNQADIAQNQANIQDLAAYNELQDAYAKQQTEAIDALNK |
| | ASSENTQNIEDLAAYNELQDAYAKQQTEAIDALNKASSENTQNIEDLAAYNELQD |
| | AYAKQQTEAIDALNKASSENTQNIAKNQADIANNINNIYELAQQQDQHSSDIKTL |
| | AKASAANTDRIAKNKADADASFETLTKNQNTLIEKDKEHDKLITANKTAIDENKA |
| | SADTKFAATADAITKNGNAITKNAKSITDLGTKVDAFDGRVTALDTKVNAFDGRI |
| | TALDSKVENGMAAQAALSGLFQPYSVGKFNATAALGGYGSKSAVAIGAGYRVN |
| | PNLAFKAGAAINTSGNKKGSYNIGVNYEF (687 aa) |
| Finnish 353 | MKTMKLLPLKIAVTSAMIVGLGMASTANAQQQKSPKTETFLPNIFFNEYADDLDT |
| (SEQ ID | LYHNMILGDTAITHDDQYKFYADDATEVPDSLFFNKILHDQLLYGFKEGDKIIPLD |
| NO: 6) | ENGKPVYKLDKRLENGVQKTVYSVTTKTATADDVNSAYSRGIQGDIDDLYEANK |
| | ENVNRLIEHGDKIFANEESVQYLNREVQNNIENIHELAQQQDQHSSDIKTLKKNVE |
| | KDLLDLSGRLIAQKEDIAQNQTDIQDLATYNELQDQYAQKQTEAIDALNKASSEN |
| | TQNIAKNSNHIKTLENNIEEGLLELSGHLIDQKADLTKDIKALESNVEEGLLDLSGR |
| | LIDQKADIAQNQANIQDLAAYNELQDAYAKQQTEAIDALNKASSENTQNIEDLAA |
| | YNELQDAYAKQQTEAIDALNKASSENTQNIEDLAAYNELQDAYAKQQTEAIDAL |
| | NKASSENTQNIAKNQADIANNINNIYELAQQQDQHSSDIKTLAKASAANTDRIAK |
| | NKADADASFETLTKNQNTLIEKDKEHDKLITANKTAIDANKASADTKFAATADAI |
| | TKNGNAITKNAKSITDLGTKVDGFDGRVTALDTKVNALDTKVNAFDGRITALDS |
| | KVENGMAAQAALSGLFQPYSVGKFNATAALGGYGSKSAVAIGAGYRVNPNLAF |
| | KAGAAINTSGNKKGSYNIGVNYEF (683 amino acids) |
| Finnish 358 | MKTMKLLPLKIAVTSAMMVGLGMASTANAQQQKSPKTEIFLPNLFDNDNTELTD |
| (SEQ ID | PLYHNMILGNTALLTQENQYKFYADDGNGVPDSLLFNKILHDQLLHGFKEGGTIIP |
| NO: 7) | LDENGKPVYKLDSIVEQGKTKTVYSVTTKTATADDVNSAYSRGIQGDIDDLYEAN |
| | KENVNRLIEHGDKIFANEESVQYLNREVQNNIENIHELAQQQDQHSSDIKTLKKNV |
| | EKDLLDLSGRLIAQKEDIAQNQTDIQDLATYNELQDQYAQKQTEAIDALNKASSE |
| | NTQNIAKNSNHIKTLENNIEEGLLELSGHLIDQKADLTKDIKALESNVEEGLLDLSG |
| | RLIDQKADIAQNQANIQDLAAYNELQDAYAKQQTEAIDALNKASSENTQNIEDLA |
| | AYNELQDAYAKQQTEAIDALNKASSENTQNIEDLAAYNELQDAYAKQQTEAIDA |
| | LNKASSENTQNIAKNQADIANNINNIYELAQQQDQHSSDIKTLAKASAANTDRIAK |
| | NKADADASFETLTKNQNTLIEKDKEHDKLITANKTAIDANKASADTKFAATADAI |
| | KVENGMAAQAALSGLFQPYSVGKFNATAALGGYGSKSAVAIGAGYRVNPNLAF |
| | KAGAAINTSGNKKGSYNIGVNYEF (684 amino acids) |
| Finnish 216 | MKTMKLLPLKIAVTSAMIIGLGAASTANAQQQQKTKTEVFLPNLFDNDYYDLTDP |
| (SEQ ID | LYHSMILGDTATLFDQQDNSKSQLKFYSNDKDSVPDSLLFSKLLHEQQLNGFKAG |
| NO: 8) | DTIIPLDKDGKPVYTQDTRTKDGKVETVYSVTTKIATQDDVEQSAYSRGIQGDIDD |
| | LYDINREVNEYLKATHDYNERQTEAIDALNKASSANTDRIDTAEERIDKNEYDIKA |
| | LESNVGKDLLDLSGRLIAQKEDIDNNINHIYELAQQQDQHSSDIKTLKNNVEEGLL |
| | ELSGHLIDQKADLTKDIKTLENNIEEGLLELSGHLIDQKADLTKDIKTLENNIEEGL |
| | LELSGHLIDQKADIAQNQANIQDLAAYNELQDQYAQKQTEAIDALNKASSENTQN |
| | IEDLAAYNELQDAYAKQQTEAIDALNKASSENTQNIEDLAAYNELQDAYAKQQT |
| | EAIDALNKASSENTQNIAKNQADIANNINNIYELAQQQDQHSSDIKTLAKVSAANT |
| | DRIAKNKADADASFETLTKNQNTLIEKDKEHDKLITANKTAIDANKASADTKFAA |
| | TADAITKNGNAITKNAKSITDLGTKVDGFDGRVTALDTKVNAFDGRITALDSKVE |
| | NGMAAQAALSGLFQPYSVGKFNATAALGGYGSKSAVAIGAGYRVNPNLAFKAG |
| | AAINTSGNKKGSYNIGVNYEF (684 amino acids) |
| Dutch H2 | MKTMKLLPLKIAVTSAMMVGLGMASTANAQQQKSPKTEIFLPNLFDNDNTELTD |
| (SEQ ID | PLYHNMILGNTALLTQENQYKFYADDGNGVPDSLLFNKILHDQLLHGFKKGDTII |
| NO: 9) | PLDENGKPVYKLDSIVEQGKTKTVYSVTTKTATADDVNSAYSRGIQGDIDDLYEA |
| | NKENVNRLIEHGDKIFANEESVQYLNREVQNNIENIYELVQQQDQHSSDIKTLKKN |
| | VEKDLLDLSGRLIAQKEDIAQNQTDIQDLATYNELQDQYAQKQTEAIDALNKASS |
| | ENTQNIAKNSNHIKTLENNIEEGLLELSGHLIDQKADLTKDIKALESNVEEGLLDLS |
| | GRLIDQKADIAQNQANIQDLAAYNELQDAYAKQQTEAIDALNKASSENTQNIEDL |
| | AAYNELQDAYAKQQTEAIDALNKASSENTQNIEDLAAYNELQDAYAKQQTEAID |
| | ALNKASSENTQNIAKNQADIANNINNIYELAQQQDQHSSDIKTLAKASAANTDRIA |
| | KNKADADASFETLTKNQNTLIEKDKEHDKLITANKTAIDANKASADTKFAATAD |
| | AITKNGNAITKNAKSITDLGTKVDGFDGRVTALDTKVNALDTKVNAFDGRITALD |
| | SKVENGMAAQAALSGLFQPYSVGKFNATAALGGYGSKSAVAIGAGYRVNPNLA |
| | FKAGAAINTSGNKKGSYNIGVNYEF (684 amino acids) |
| Dutch F10 | MKTMKLLPLKIAVTSAMIIGLGAASTANAQLAEQFFPNIFSNHAPVKQHYHNVVV |
| (SEQ ID | GDTSIVENLQDSDDTQLKFYSNDEYSVPDSLLFNKMLHEQQLNGFKKGDTIIPLDE |
| NO: 10) | NGKPVYKVDYKLDGQEPRRVYSVTTKIATQDDVDNSPYSRGIQGDIDDLYEANK |
| | ENVNRLIEHGDKIFANEESVQYLNKEVQNNIENIYELAQQQDQHSSDIKTLKKNVE |
| | EGLLELSGHLIDQKADLTKDIKTLESNVEEGLLELSGHLIDQKADIAKNQADIAQN |
| | QANIQDLAAYNELQDAYAKQQTEAIDALNKASSENTQNIEDLAAYNELQDAYAK |
| | QQTEAIDALNKASSENTQNIAKNQADIANNINNIYELAQQQDQHSSDIKTLAKASA |
| | ANTDRIAKNKADADASFETLTKNQNTLIEKDKEHDKLITANKTAIDANKASADTK |
| | FAATADAITKNGNAITKNAKSITDLGTKVDAFDGRVTALDTKVNAFDGRITALDS |
| | KVENGMAAQAALSGLFQPYSVGKFNATAALGGYGSKSAVAIGAGYRVNPNLAF |
| | KAGAAINTSGNKKGSYNIGVNYEF (574 amino acids) |
| Norwegian | MKTMKLLPLKIAVTSALIVGLGAASTANAQQQPQTETFFPNIFFNENHDALDDVY |
| 1 | HNMILGDTAITQDNQYKFYADAISEVPDSLLFNKILHDQQLNGFKEGDTIIPLDEN |
| (SEQ ID | GKPVYKLDEKVENGVKKSVYSVTTKTATRADVEQSAYSRGIQGDIDDLYEANKE |
| NO: 11) | NVNRLIEHGDKIFANEESVQYLNKEVQNNIENIHELAQQQDQHSSDIKTLKKNVEE |
| | GLLELSGHLIDQKADLTKDIKTLESNVEEGLLDLSGRLLDQKADIAQNQANIQDLA |
| | AYNELQDAYAKQQTEAIDALNKASSENTQNIEDLAAYNELQDAYAKQQTEAIDA |
| | LNKASSENTQNIEDLAAYNELQDAYAKQQTEAIDALNKASSENTQNIEDLAAYNE |
| | LQDAYAKQQTEAIDALNKASSENTQNIEDLAAYNELQDAYAKQQTEAIDALNKA |
| | SSENTQNIAKNQADIANNINNIYELAQQQDQHSSDIKTLAKASAANTDRIAKNKA |
| | DADASFETLTKNQNTLIEKDKEHDKLITANKTAIDANKASADTKFAATADAITKN |
| | GNAITKNAKSITDLGTKVDAFDGRVTALDTKVNALDTKVNAFDGRITALDSKVE |
| | NGMAAQAALSGLFQPYSVGKFNATAALGGYGSKSAVAIGAGYRVNPNLAFKAG |
| | AAINTSGNKKGSYNIGVNYEF (678 amino acids) |
| Norwegian | MKTMKLLPLKIAVTSAMIVGLGAASTANAQQQQQPRTETFFPNIFFNENHDALDD |
| 13 | VYHNMILGDTAITQDNQYKFYADAISEVPDSLLFNKILHDQQLNGFKEGDTIIPLD |
| (SEQ ID | ENGKPVYKLDEKVENGVKKSVYSVTTKTATRADVEQSAYSRGIQGDIDDLYEAN |
| NO: 12) | KENVNRLIEHGDKIFANEESVQYLNREVQNNIENIHELAQQQDQHSSDIKTLKKNV |
| | EKDLLDLSGRLIAQKEDIAQNQTDIQDLATYNELQDQYAQKQTEAIDALNKASSE |
| | NTQNIAKNSNHIKTLENNIEEGLLELSGHLIDQKADLTKDIKTLENNIEEGLLELSG |
| | HLIDQKADLTKDIKALESNVEEGLLDLSGRLLDQKADIAQNQANIQDLAAYNELQ |
| | DQYAQKQTEAIDALNKASSENTQNIEDLAAYNELQDAYAKQQTEAIDALNKASS |
| | ENTQNIAKNQADIANNINNIYELAQQQDQHSSDIKTLAKASAANTDRIAKNKADA |
| | DASFETLTKNQNTLIEKDKEHDKLITANKTAIDTNKASADTKFAATADAITKNGN |
| | AITKNAKSITDLGTKVDGFDGRVTALDTKVNALDTKVNAFDGRITALDSKVENG |
| | MAAQAALSGLFQPYSVGKFNATAALGGYGSKSAVAIGAGYRVNPNLAFKAGAAI |
| | NTSGNKKGSYNIGVNYEF (678 amino acids) |
| Norwegian | MKTMKLLPLKIAVTSALIVGLGAASTANAQLVERFFPNIFLDKPLAKQHYHNVVV |
| 20 | GDTSIVSDLQSNSDQLKFYSDDEGLVPDSLLFNKMLHEQLLNGFKEGDTIIPLDEN |
| (SEQ ID | GKPVYKVDYKLDGKEPRKVYSVTTKIATAEDVATSSYANGIQKDIDDLYDFDHQ |
| NO: 13) | VTERLTQHGKTIYRNGERILANEESVQYLNKEVQNNIEHIYELAQQQDQHSSDIKT |
| | LESNVEKGLLELSGHLIDQKADLTKDIKTLENNVEEGLLDLSGRLIDQKADIAQNQ |
| | ANIQDLAAYNELQDQYAQKQTEAIDALNKASSENTQNIEDLAAYNELQDAYAKQ |
| | QTEAIDALNKASSENTQNIAKNQADIANNINNIYELAQQQDQHSSDIKTLAKASAA |
| | NTDRIAKNKADADASFETLTKNQNTLIEKDKEHDKLITANKTAIDTNKASADTKF |
| | AATADAITKNGNAITKNAKSITDLGTKVDGFDSRVTALDTKVNALDTKVNALDT |
| | KVNAFDGRITALDSKVENGMAAQAALSGLFQPYSVGKFNATAALGGYGSKSAV |
| | AIGAGYRVNPNLAFKAGAAINTSGNKKGSYNIGVNYEF (587 amino acids) |
| Norwegian | MKTMKLLPLKIAVTSAMIVGLGAASTANAQQQQQPRTETFFPNIFFNENHDALDD |
| 25 | VYHNMILGDTAITQDNQYKFYADAISEVPDSLLFNKILHDQQLNGFKEGDTIIPLD |
| (SEQ ID | ENGKPVYKLDEKVENGVKKSVYSVTTKTATRADVEQSAYSRGIQGDIDDLYEAN |
| NO: 14) | KENVNRLIEHGDKIFANEESVQYLNREVQNNIENIHELAQQQDQHSSDIKTLKKNV |
| | EKDLLDLSGRLIAQKEDIAQNQTDIQDLATYNELQDQYAQKQTEAIDALNKASSE |
| | NTQNIAKNSNHIKTLENNIEEGLLELSGHLIDQKADLTKDIKTLENNIEEGLLELSG |
| | HLIDQKADLTKDIKALESNVEEGLLDLSGRLLDQKADIAQNQANIQDLAAYNELQ |
| | DQYAQKQTEAIDALNKASSENTQNIEDLAAYNELQDAYAKQQTEAIDALNKASS |
| | ENTQNIAKNQADIANNINNIYELAQQQDQHSSDIKTLAKASAANTDRIAKNKADA |
| | DASFETLTKNQNTLIEKDKEHDKLITANKTAIDTNKASADTKFAATADAITKNGN |
| | AITKNAKSITDLGTKVDGFDGRVTALDTKVNALDTKVNAFDGRITALDSKVENG |
| | MAAQAALSGLFQPYSVGKFNATAALGGYGSKSAVAIGAGYRVNPNLAFKAGAAI |
| | NTSGNKKGSYNIGVNYEF (678 amino acids) |
| Norwegian | MKTMKLLPLKIAVTSALIVGLGAASTANAQVRDKSLEDIEALLGKIDISKLEKEKK |
| 27 | QQTELQKYLLLSQYANVLTMEELNKNVEKNTNSIEALGYEIGWLENDIADLEEGV |
| (SEQ ID | EELTKNQNTLIEKDEEHDRLIAQNQADIKTLENNVVEELFNLSDRLIDQEADIAKN |
| NO: 15) | NASIEELYDFDNEVAERIGEIHAYTEEVNKTLEKLITNSVKNTDNIDKNKADIQALE |
| | NNVEEGLLELSGHLIDQKADLTKDIKALESNVEEGLLDLSGRLLDQKADIAKNQA |
| | DIAQNQTDIQDLAAYNELQDQYAQKQTEAIDALNKASSENTQNIEDLAAYNELQD |
| | AYAKQQTEAIDALNKASSENTQNIEDLAAYNELQDAYAKQQTEAIDALNKASSE |
| | NTQNIAKNQADIANNINNIYELAQQQDQHSSDIKTLAKVSAANTDRIAKNKADAD |
| | ASFETLTKNQNTLIEKDKEHDKLITANKTAIDANKASADTKFAATADAITKNGNAI |
| | TKNAKSITDLGTKVDGFDSRVTALDTKVNAFDGRITALDSKVENGMAAQAALSG |
| | LFQPYSVGKFNATAALGGYGSKSAVAIGAGYRVNPNLAFKAGAAINTSGNKKGS |
| | YNIGVNYEF (616 amino acids) |
| Norwegian | MKTMKLLPLKIAVTSALIVGLGAASTANAQATETFLPNLFDNDYTETTDPLYHGM |
| 36 | ILGNTAITQDTQYKFYAENGNEVPDSLFFNKILHDQQLNGFKEGDTIIPLDENGKP |
| (SEQ ID | VYKLDEITENGVKRKVYSVTTKTATREDVEQSAYSRGIQGDIDDLYEANKENVNR |
| NO: 16) | LIEHGDKIFANEESVQYLNKEVQNNIENIHELAQQQDQHSSDIKTLKKNVEEGLLE |
| | LSGHLIDQKADLTKDIKALESNVEEGLLDLSGHLIDQKADLTKDIKALESNVEEGL |
| | LDLSGRLLDQKADIAKNQADIAQNQTDIQDLAAYNELQDQYAQKQTEAIDALNK |
| | ASSENTQNIEDLAAYNELQDQYAQKQTEAIDALNKASSENTQNIEDLAAYNELQD |
| | QYAQKQTEAIDALNKASSENTQNIEDLAAYNELQDQYAQKQTEAIDALNKASSE |
| | NTQNIAKNQADIANNINNIYELAQQQDQHSSDIKTLAKASAANTDRIAKNKADAD |
| | ASFETLTKNQNTLIEKDKEHDKLITANKTAIDANKASADTKFAATADAITKNGNAI |
| | TKNAKSITDLGTKVDGFDGRVTALDTKVNALDTKVNAFDGRITALDSKVENGMA |
| | AQAALSGLFQPYSVGKFNATAALGGYGSKSAVAIGAGYRVNPNLAFKAGAAINT |
| | SGNKKGSYNIGVNYEF (676 amino acids) |
| BC5SV | MKTMKLLPLKIAVTSALIVGLGAASTANAQNGTSTKLKNLKEYAQYLDNYAQYL |
| (SEQ ID | DDDIDDLDKEVGELSQNIAKNQANIKDLNKKLSRDIDSLREDVYDNQYEIVNNQA |
| NO: 17) | DIEKNQDDIKELENNVGKELLNLSGRLLDQKADIDNNINNIYELAQQQDQHSSDIK |
| | TLKKNVEEGLLELSGHLIDQKSDIAQNQTDIQDLATYNELQDQYAQKQTEAIDAL |
| | NKASSENTQNIEDLAAYNELQDAYAKQQTEAIDALNKASSENTQNIQDLAAYNEL |
| | QDAYAKQQTEAIDALNKASSENTQNIEDLAAYNELQDAYAKQQTEAIDALNKAS |
| | SENTQNIEDLAAYNELQDAYAKQQTEAIDALNKASSENTQNIEDLAAYNELQDAY |
| | AKQQTEAIDALNKASSENTQNIAKNQADIANNINNIYELAQQQDQHSSDIKTLAK |
| | ASAANTDRIAKNKADADASFETLTKNQNTLIEKDKEHDKLITANKTAIDANKASA |
| | DTKFAATADAITKNGNAITKNAKSITDLGTKVDAFDGRVTALDTKVNAFDGRITA |
| | LDSKVENGMAAQAALSGLFQPYSVGKFNATAALGGYGSKSAVAIGAGYRVNPN |
| | LAFKAGAAINTSGNKKGSYNIGVNYEF (629 amino acids) |
| Norwegian | MKTMKLLPLKIAVTSAMIVGLGMASTANAQQQRSPKTETFLPNIFFNEYADDLDT |
| 14 | LYHNMILGDTAITHDDQYKFYADDATEVPDSLFFNKILHDQLLYGFKEGDKIIPLD |
| (SEQ ID | ENGKPVYKLDKRLDNGVQKTVYSVTTKTATADDVNSAYSRGIQGDIDDLYEANK |
| NO: 18) | ENVNRLIEHGDKIFANEESVQYLNKEVQNNIENIHELAQQQDQHSSDIKTLKKNVE |
| | EGLLELSGHLIDQKTDIAQNQTDIQDLATYNELQDQYAQKQTEAIDALNKASSEN |
| | TQNIAKNSNRIKALENNIEEGLLELSGHLIDQKADLTKDIKALESNVEEGLLDLSGR |
| | LIDQKADIAQNQANIQDLAAYNELQDAYAKQQTEAIDALNKASSENTQNIEDLAA |
| | YNELQDAYAKQQTEAIDALNKASSENTQNIEDLAAYNELQDAYAKQQTEAIDAL |
| | NKASSENTQNIAKNQADIANNINNIYELAQQQDQHSSDIKTLAKASAANTDRIAK |
| | NKADADASFETLTKNQNTLIEKDKEHDKLITANKTAIDANKASADTKFAATADAI |
| | TKNGNAITKNAKSITDLGTKVDGFDGRVTALDTKVNALDTKVNAFDGRITALDS |
| | KVENGMAAQAALSGLFQPYSVGKFNATAALGGYGSKSAVAIGAGYRVNPNLAF |
| | KAGAAINTSGNKKGSYNIGVNYEF (683 amino acids) |
| Norwegian | MKTMKLLPLKIAVTSAMIVGLGAASTANAQAQSNRSLDQVQALLRGIDETKIKKE |
| 3 | IQQSQQPELNKYLTFNQLANALNIEELNNNVQKNTQRLDSAATLYGDLSKTVPKS |
| (SEQ ID | IKENKESIKENKESIKENKESIKENKESIKENKESIKENKESITTLTRKSFQNQVDIVR |
| NO: 19) | NNASIEDLYAYGQEVAKSIGEIHAYTEEVNKTLENLITNSVENTNNITKNKADIQA |
| | LENNVVEELFNLSGRLIDQKADIDNNINNIYELAQQQDQHSSDIKTLKKNVEEGLL |
| | ELSGHLIDQKADLTKDIKTLESNVEEGLLDLSGRLLDQKADIAQNQANIQDLAAY |
| | NELQDAYAKQQTEAIDALNKASSENTQNIEDLAAYNELQDAYAKQQTEAIDALN |
| | KASSENTQNIEDLAAYNELQDAYAKQQTEAIDALNKASSENTQNIEDLAAYNELQ |
| | DAYAKQQTEAIDALNKASSENTQNIAKNQADIANNINNIYELAQQQDQHSSDIKT |
| | LAKASAANTDRIAKNKADADASFETLTKNQNTLIEKDKEHDKLITANKTVIDANK |
| | ASADTKFAATADAITKNGNAITKNAKSITDLGTKVDGFDGRVTALDTKVNALDT |
| | KVNAFDGRITALDSKVENGMAAQAALSGLFQPYSVGKFNATAALGGYGSKSAV |
| | AIGAGYRVNPNLAFKAGAAINTSGNKKGSYNIGVNYEF (700 amino acids) |
| Finnish | MKTMKLLPLKIAVTSALIVGLGAASTANAQATETFLPNLFDNDYIETTDPLYHGMI |
| 414 | LGNTAITQDTQYKFYAENGNEVPDSLFFNKILHDQQLNGFKEGDTIIPLDENGKPV |
| (SEQ ID | YKLDEITENGVKRKVYSVTTKTATREDVEQSAYSRGIQGDIDDLYEANKENVNRL |
| NO: 20) | IEHGDKIFANEESVQYLNKEVQNNIENIHELAQQQDQHSSDIKTLKKNVEEGLLEL |
| | SGHLIDQKADLTKDIKTLENNVEEGLLELSGHLIDQKADLTKDIKALESNVEEGLL |
| | DLSGRLLDQKADIAKNQADIAQNQTDIQDLAAYNELQDQYAQKQTEAIDALNKA |
| | SSENTQNIEDLAAYNELQDQYAQKQTEAIDALNKASSENTQNIEDLAAYNELQDQ |
| | YAQKQTEAIDALNKASSENTQNIEDLAAYNELQDQYAQKQTEAIDALNKASSENT |
| | QNIAKNQADIANNINNIYELAQQQDQHSSDIKTLAKASAANTDRIAKNKADADAS |
| | FETLTKNQNTLIEKDKEHDKLITANKTAIDANKASADTKFAATADAITKNGNAITK |
| | NAKSITDLGTKVDGFDGRVTALDTKVNALDTKVNAFDGRITALDSKVENGMAA |
| | QAALSGLFQPYSVGKFNATAALGGYGSKSAVAIGAGYRVNPNLAFKAGAAINTS |
| | GNKKGSYNIGVNYEF (676 amino acids) |
| Japanese | MKTMKLLPLKIAVTSAMIIGLGAASTANAQLAEQFFPNIFSNHAPVKQHYHNVVV |
| Z7476 | GDTSIVENLQDSDDTQLKFYSNDEYSVPDSLLFNKMLHEQQLNGFKKGDTIIPLDE |
| (SEQ ID | NGKPVYKVDYKLDGQEPRRVYSVTTKIATQDDVDNSPYSRGIQGDIDDLYEANK |
| NO: 21) | ENVNRLIEHGDKIFANEESVQYLNKEVQNNIENIYELAQQQDQHSSDIKTLKKNVE |
| | EGLLELSGRLIDQKADIAQNQANIQDLAAYNELQDQYAQKQTEAIDALNKASSEN |
| | TQNIEDLAAYNELQDAYAKQQTEAIDALNKASSENTQNIEDLAAYNELQDAYAK |
| | QQTEAIDALNKASSENTQNIEDLAAYNELQDAYAKQQTEAIDALNKASSENTQNI |
| | EDLAAYNELQDAYAKQQTEAIDALNKASSENTQNIEDLAAYNELQDAYAKQQTE |
| | AIDALNKASSENTQNIAKNQADIANNINNIYELAQQQDQHSSDIKTLAKVSAANTD |
| | RIAKNKADADASFETLTKNQNTLIEKDKEHDKLITANKTAIDANKASADTKFAAT |
| | ADAITKNGNAITKNAKSITDLGTKVDGFDGRVTALDTKVNAFDGRITALDSKVEN |
| | GMAAQAALSGLFQPYSVGKFNATAALGGYGSKSAVAIGAGYRVNPNLAFKAGA |
| | AINTSGNKKGSYNIGVNYEF (678 amino acids) |
| Belgian | MKTMKLLPLKIAVTSAMIIGLGAASTANAQSRDRSLEDIQDSISKLVQDDINTLKQ |
| Z7530 | DQQKMNKYLLLNQLANTLITDELNNNVIKNTNSIEALGDEIGWLENDIADLEEGV |
| (SEQ ID | EELTKNQNTLIEKDEEHDRLIAQNQADIQTLENNVVEELFNLSGRLIDQEADIAKN |
| NO: 22) | NASIEELYDFDNEVAERIGEIHAYTEEVNKTLENLITNSVKNTDNIDKNKADIDNNI |
| | NHIYELAQQQDQHSSDIKTLKNNVEEGLLELSGHLIDQKADLTKDIKALESNVEEG |
| | LLDLSGRLLDQKADLTKDIKALESNVEEGLLDLSGRLLDQKADIAQNQTDIQDLA |
| | AYNELQDQYAQKQTEAIDALNKASSENTQNIEDLAAYNELQDAYAKQQTEAIDA |
| | LNKASSENTQNIAKNQADIANNINNIYELAQQQDQHSSDIKTLAKASAANTNRIAT |
| | AELGIAENKKDAQIAKAQANANKTAIDENKASADTKFAATADAITKNGNAITKN |
| | AKSITDLGTKVDGFDGRVTALDTKVNAFDGRITALDSKVENGMAAQAALSGLFQ |
| | PYSVGKFNATAALGGYGSKSAVAIGAGYRVNPNLAFKAGAAINTSGNKKGSYNI |
| | GVNYEF (613 amino acids) |
| German | MKTMKLLPLKIAVTSALIVGLGAASTANAQATNKDITLEDVLKSIEEIDPYELRDYI |
| Z8063 | EYPTAIERFLLLSQYGNTLTLEEFDNDIELLDQDVEDLEESVTELAKNQNSLIEQGE |
| (SEQ ID | AIKEDLQGLADFVERQEDKILQNETSIKKNTQRNLVNGFEIEKNKDAIAKNNESIE |
| NO: 23) | DLYDFGHEVAKSIGEIHAHNEAQNETLKDLITNSVKNTDNITKNKADIQALESNVE |
| | KGLLELSGHLIDQKADIDNNINNIHELAQQQDQHSSDIKTLKKNVEEGLLELSGHLI |
| | DQKSDIAQNQANIQDLATYNELQDQYAQKQTEAIDALNKASSENTQNIEDLAAY |
| | NELQDAYAKQQTEAIDALNKASSENTQNIAKNQADIANNINNIYELAQQQDQHSS |
| | DIKTLAKASAANTDRIAKNKADADASFETLTKNQNTLIEKDKEHDKLITANKTAID |
| | ANKASADTKFAATADAITKNGNAITKNAKSITDLGTKVDGFDSRVTALDTKVNA |
| | FDGRITALDSKVENGMAAQAALSGLFQPYSVGKFNATAALGGYGSKSAVAIGAG |
| | YRVNPNLAFKAGAAINTSGNKKGSYNIGVNYEF (589 amino acids) |
| American | MKTMKLLPLKIAVTSAMMVGLGMASTANAQQQKSPKTEIFLPNLFDNDNTELTD |
| O12E | PLYHNMILGNTALLTQENQYKFYADDGNGVPDSLLFNKILHDQLLHGFKEGDTIIP |
| (SEQ ID | LDENGKPVYKLDSIVEQGKTKTVYSVTTKTATADDVNSAYSRGIQGDIDDLYEAN |
| NO: 24) | KENVNRLIEHGDKIFANEESVQYLNREVQNNIENIHELAQQQDQHSSDIKTLKKNV |
| | EKDLLDLSGRLIAQKEDIAQNQTDIQDLATYNELQDQYAQKQTEAIDALNKASSE |
| | NTQNIAKNSNHIKTLENNIEEGLLELSGHLIDQKADLTKDIKALESNVEEGLLDLSG |
| | RLIDQKADIAQNQANIQDLAAYNELQDAYAKQQTEAIDALNKASSENTQNIEDLA |
| | AYNELQDAYAKQQTEAIDALNKASSENTQNIEDLAAYNELQDAYAKQQTEAIDA |
| | LNKASSENTQNIAKNQADIANNINNIYELAQQQDQHSSDIKTLAKASAANTDRIAK |
| | NKADADASFETLTKNQNTLIEKDKEHDKLITANKTAIDANKASADTKFAATADAI |
| | TKNGNAITKNAKSITDLGTKVDGFDGRVTALDTKVNALDTKVNAFDGRITALDS |
| | KVENGMAAQAALSGLFQPYSVGKFNATAALGGYGSKSAVAIGAGYRVNPNLAF |
| | KAGAAINTSGNKKGSYNIGVNYEF (684 amino acids) |
| Greek | MKTMKLLPLKIAVTSALIVGLGAASTANAQQQQKTKTEVFLPNLFYNDYIEETDL |
| MC317 | LYHNMILGDTAALVDRQNYSNSQLKFYSNDEESVPDSLLFSKMLNNQQLNGFKA |
| (SEQ ID | GDIIIPVDANGQVIYQKDTRVEGGKTRTVLSVTTKIATQQDVDSAYSRGIQGKVND |
| NO: 25) | LDDEMNFLNHDITSLYDVTANQQDDIKGLKKGVKDLKKGVKGLNKELKELDKE |
| | VGVLSRDIGSLNDDVAQNNESIEDLYDFSQEVADSIGEIHAHNKAQNETLQDLITN |
| | SVENTNNITKNKADIQALENNVVEELFNLSGRLIDQKADLTKDIKTLESNVEEGLL |
| | ELSGHLIDQKADIAKNQADIAQNQANIQDLAAYNELQDAYAKQQTEAIDALNKA |
| | SSENTQNIEDLAAYNELQDAYAKQQTEAIDALNKASSENTQNIAKNQADIANNIN |
| | NIYELAQQQDQHSSDIKTLAKASAANTDRIAKNKADADASFETLTKNQNTLIEKD |
| | KEHDKLITANKTAIDENKASADTKFAATADAITKNGNAITKNAKSITDLGTKVDG |
| | FDGRVTALDTKVNAFDGRITALDSKVENGMAAQAALSGLFQPYSVGKFNATAAL |
| | GGYGSKSAVAIGAGYRVNPNLAFKAGAAINTSGNKKGSYNIGVNYEF (650 amino |
| | acids) |
| American | MKTMKLLPLKIAVTSALIVGLGAVSTTNAQAQSRSLDQIQTKLADLAGKIAAGKN |
| V1122 | GGGQNNQNNQNDINKYLFLSQYANILTMEELNNNVVKNSSSIETLETDFGWLEND |
| (SEQ ID | VADLEDGVEELTKNQNTLIEKDEEHDRLIAQNQADIQTLENNVVEELFNLSDRLID |
| NO: 26) | QKADIAKNQADIAQNNESIEELYDFDNEVAEKIGEIHAYTEEVNKTLQDLITNSVK |
| | NTDNIDKNKADIDNNINHIYELAQQQDQHSSDIKTLKNNVEEGLLELSGHLIDQKA |
| | DLTKDIKTLENNVEEGLLDLSGRLIDQKADIAKNQADIAQNQTDIQDLAAYNELQ |
| | DQYAQKQTEAIDALNKASSENTQNIEDLAAYNELQDAYAKQQTEAIDALNKASS |
| | ENTQNIAKNQADIANNINNIYELAQQQDQHSSDIKTLAKASAANTDRIAKNKADA |
| | DASFETLTKNQNTLIEKDKEHDKLITANKTAIDENKASADTKFAATADAITKNGN |
| | AITKNAKSITDLGTKVDGFDGRVTALDTKVNAFDGRITALDSKVENGMAAQAAL |
| | SGLFQPYSVGKFNATAALGGYGSKSAVAIGAGYRVNPNLAFKAGAAINTSGNKK |
| | GSYNIGVNYEF (616 amino acids) |
| American | MKTMKLLPLKIAVTSALIVGLGTASTANAQVASPANQKIQQKIKKVRKELRQDIK |
| P44 | SLRNDIDSNTADIGSLNDDVADNQDDILDNQADIAKNQDDIEKNQADIKELDKEV |
| (SEQ ID | GVLSREIGSLNDDIADNYTDIIDNYTDIIDNQANIAKNQDDIEKNQADIKELDKEVG |
| NO: 27) | VLSREIGSLNDDVADNQDDIAKNQADIQTLENNVEEGLLELSGHLLDQKADIDNNI |
| | NNIYELAQQQDQHSSDIKTLKKNVEEGLLELSGHLIDQKTDIAQNQANIQDLATY |
| | NELQDQYAQEQTEAIDALNKASSENTQNIAKNSNRIKALESNVEEGLLELSGHLID |
| | QKADLTKDIKALESNVEEGLLELSGHLIDQKADIAQNQANIQDLAAYNELQDQYA |
| | QKQTEAIDALNKASSENTQNIEDLAAYNELQDAYAKQQTEAIDALNKASSENTQN |
| | IAKNQADIANNINNIYELAQQQDQHSSDIKTLAKASAANTDRIAKNKADADASFE |
| | TLTKNQNTLIEKDKEHDKLITANKTAIDANKVSADTKFAATADAITKNGNAITKN |
| | AKSITDLGTKVDAFDSRVTALDTKVNAFDGRITALDSKVENGMAAQAALSGLFQ |
| | PYSVGKFNATAALGGYGSKSAVAIGAGYRVNPNLAFKAGAAINTSGNKKGSYNI |
| | GVNYEF |
| | (668 amino acids) |
| American | MKTMKLLPLKIAVTSAMIVGLGATSTVNAQVVEQFFPNIFFNENHDELDDAYHN |
| V1171 | MILGDTAIVSNSQDNSTQLKFYSNDEDSVPDSLLFSKLLHEQQLNGFKAGDTIIPLD |
| (SEQ ID | KDGKPVYTKDTRTKDGKVETVYSVTTKIATQDDVEQSAYSRGIQGDIDDLYDINR |
| NO: 28) | EVNEYLKATHDYNERQTEAIDALNKASSANTDRIDTAEERIDKNEYDIKALESNV |
| | EEGLLELSGHLIDQKADLTKDIKALESNVEEGLLELSGHLIDQKADLTKDIKALES |
| | NVEEGLLDLSGRLIDQKADIAQNQANIQDLAAYNELQDAYAKQQTEAIDALNKA |
| | SSENTQNIEDLAAYNELQDAYAKQQTEAIDALNKASSENTQNIEDLAAYNELQDA |
| | YAKQQTEAIDALNKASSENTQNIEDLAAYNELQDAYAKQQTEAIDALNKASSENT |
| | QNIAKNQADIANNINNIYELAQQQDQHSSDIKTLAKASAANTDRIAKNKADADAS |
| | FETLTKNQNTLIEKDKEHDKLITANKTAIDANKASADTKFAATADAITKNGNAITK |
| | NAKSITDLGTKVDGFDGRVTALDTKVNALDTKVNAFDGRITALDSKVENGMAA |
| | QAALSGLFQPYSVGKFNATAALGGYGSKSAVAIGAGYRVNPNLAFKAGAAINTS |
| | GNKKGSYNIGVNYEF (674 amino acids) |
| American | MKTMKLLPLKIAVTSAMIIGLGAASTANAQSRDRSLEDIQDSISKLVQDDIDTLKQ |
| TTA24 | DQQKMNKYLLLNQLANTLITDELNNNVIKNTNSIEALGDEIGWLENDIADLEEGV |
| (SEQ ID | EELTKNQNTLIEKDEEHDRLIAQNQADIQTLENNVVEELFNLSGRLIDQEADIAKN |
| NO: 29) | NASIEELYDFDNEVAERIGEIHAYTEEVNKTLENLITNSVKNTDNIDKNKADIDNNI |
| | NHIYELAQQQDQHSSDIKTLKNNVEEGLLELSGHLIDQKADLTKDIKALESNVEEG |
| | LLDLSGRLLDQKADLTKDIKALESNVEEGLLDLSGRLLDQKADIAQNQTDIQDLA |
| | AYNELQDQYAQKQTEAIDALNKASSENTQNIEDLAAYNELQDAYAKQQTEAIDA |
| | LNKASSENTQNIAKNQADIANNINNIYELAQQQDQHSSDIKTLAKASAANTNRIAT |
| | AELGIAENKKDAQIAKAQANANKTAIDENKASADTKFAATADAITKNGNAITKN |
| | AKSITDLGTKVDGFDGRVTALDTKVNAFDGRITALDSKVENGMAAQAALSGLFQ |
| | PYSVGKFNATAALGGYGSKSAVAIGAGYRVNPNLAFKAGAAINTSGNKKGSYNI |
| | GVNYEF (613 amino acids) |
| American | MKTMKLLPLKIAVTSAMIVGLGATSTVNAQVVEQFFPNIFFNENHDELDDAYHN |
| O35E | MILGDTAIVSNSQDNSTQLKFYSNDEDSVPDSLLFSKLLHEQQLNGFKAGDTIIPLD |
| (SEQ ID | KDGKPVYTKDTRTKDGKVETVYSVTTKIATQDDVEQSAYSRGIQGDIDDLYDINR |
| NO: 30) | EVNEYLKATHDYNERQTEAIDALNKASSANTDRIDTAEERIDKNEYDIKALESNV |
| | EEGLLELSGHLIDQKADLTKDIKALESNVEEGLLELSGHLIDQKADLTKDIKALES |
| | NVEEGLLDLSGRLLDQKADIAKNQADIAQNQTDIQDLAAYNELQDAYAKQQTEA |
| | IDALNKASSENTQNIAKNQADIANNINNIYELAQQQDQHSSDIKTLAKASAANTDR |
| | IAKNKADADASFETLTKNQNTLIEKDKEHDKLITANKTAIDANKASADTKFAATA |
| | DAITKNGNAITKNAKSITDLGTKVDGFDGRVTALDTKVNALDTKVNAFDGRITAL |
| | DSKVENGMAAQAALSGLFQPYSVGKFNATAALGGYGSKSAVAIGAGYRVNPNL |
| | AFKAGAAINTSGNKKGSYNIGVNYEF (576 amino acids) |
| American | MKTMKLLPLKIAVTSAMMVGLGMASTANAQQQKSPKTEIFLPNLFDNDNTELTD |
| SP12-6 | PLYHNMILGNTALLTQENQYKFYADDGNGVPDSLLFNKILHDQLLHGFKEGDTIIP |
| (SEQ ID | LDENGKPVYKLDSIVEQGKTKTVYSVTTKTATADDVNSAYSRGIQGDIDDLYEAN |
| NO: 31) | KENVNRLIEHGDKIFANEESVQYLNREVQNNIENIHELAQQQDQHSSDIKTLKKNV |
| | EKDLLDLSGRLIAQKEDIAQNQTDIQDLATYNELQDQYAQKQTEAIDALNKASSE |
| | NTQNIAKNSNHIKTLENNIEEGLLELSGHLIDQKADLTKDIKALESNVEEGLLDLSG |
| | RLIDQKADIAQNQANIQDLAAYNELQDAYAKQQTEAIDALNKASSENTQNIEDLA |
| | AYNELQDAYAKQQTEAIDALNKASSENTQNIEDLAAYNELQDAYAKQQTEAIDA |
| | LNKASSENTQNIAKNQADIANNINNIYELAQQQDQHSSDIKTLAKASAANTDRIAK |
| | NKADADASFETLTKNQNTLIEKDKEHDKLITANKTAIDANKASADTKFAATADAI |
| | TKNGNAITKNAKSITDLGTKVDGFDGRVTALDTKVNALDTKVNAFDGRITALDS |
| | KVENGMAAQAALSGLFQPYSVGKFNATAALGGYGSKSAVAIGAGYRVNPNLAF |
| | KAGAAINTSGNKKGSYNIGVNYEF (684 amino acids) |
| American | MKTMKLLPLKIAVTSAMIIGLGAASTANAQATETFLPNLFDNDYTETTDPLYHGM |
| SP12-5 | ILGNTAITQDTQYKFYAENGNEVPDSLFFNKILHDQQLNGFKEGDTIIPLDENGKP |
| (SEQ ID | VYKLDEITENGVKRKVYSVTTKTATREDVEQSAYSRGIQGDIDDLYEANKENVNR |
| NO: 32) | LIEHGDKIFANEESVQYLNKEVQNNIENIHELAQQQDQHSSDIKTLKKNVEEGLLE |
| | LSGRLIAQKEDIAQNQTDIQDLATYNELQDQYAQKQTEAIDALNKASSENTQNIA |
| | KNSNHIKTLENNIEEGLLELSGHLIDQKADLTKDIKALESNVEEGLLDLSGRLLDQ |
| | KADIAKNQADIAQNQTDIQDLAAYNELQDQYAQKQTEAIDALNKASSENTQNIED |
| | LAAYNELQDAYAKQQTEAIDALNKASSENTQNIEDLAAYNELQDAYAKQQTEAI |
| | DALNKASSENTQNIAKNQADIANNINNIYELAQQQDQHSSDIKTLAKASAANTDRI |
| | AKNKADADASFETLTKNQNTLIEKDKEHDKLITANKTAIDANKASADTKFAATA |
| | DAITKNGNAITKNAKSITDLGTKVDGFDGRVTALDTKVNALDTKVNAFDGRITAL |
| | DSKVENGMAAQAALSGLFQPYSVGKFNATAALGGYGSKSAVAIGAGYRVNPNL |
| | AFKAGAAINTSGNKKGSYNIGVNYEF (686 amino acids) |
| Swedish | MKTMKLLPLKIAVTSAMIIGLGAASTANAQAKNDITLEDLPYLIKKIDQNELEADI |
| BC5 | GDITALEKYLALSQYGNILALEELNKALEELDEDVGWNQNDIANLEDDVETLTKN |
| (SEQ ID | QNALAEQGEAIKEDLQGLADFVEGQEGKILQNETSIKKNTQRNLVNGFEIEKNKD |
| NO: 33) | AIAKNNESIEDLYDFGHEVAESIGEIHAHNEAQNETLKGLITNSIENTNNITKNKAD |
| | IQALENNVVEELFNLSGRLIDQKADIDNNINNIYELAQQQDQHSSDIKTLKKNVEE |
| | GLLELSGHLIDQKTDIAQNQANIQDLATYNELQDQYAQKQTEAIDALNKASSENT |
| | QNIEDLAAYNELQDAYAKQQTEAIDALNKASSENTQNIEDLAAYNELQDAYAKQ |
| | QTEAIDALNKASSENTQNIAKNQADIANNINNIYELAQQQDQHSSDIKTLAKASAA |
| | NTDRIAKNKADADASFETLTKNQNTLIEKDKEHDKLITANKTAIDANKASADTKF |
| | AATADAITKNGNAITKNAKSITDLGTKVDGFDSRVTALDTKVNAFDGRITALDSK |
| | VENGMAAQAALSGLFQPYSVGKFNATAALGGYGSKSAVAIGAGYRVNPNLAFK |
| | AGAAINTSGNKKGSYNIGVNYEF (630 amino acids) |
| American | MKTMKLLPLKIAVTSALIVGLGAASTANAQAQDRSLEQIQDKLANLVEKIEQAKS |
| 7169 | QNGQSQKDINQYLLLSQYANVLTMEELNNNVVKNSSSIETLDNDIAWLNDDLIDL |
| (SEQ ID | DKEVGVLSRDIGSLHDDVAQNQADIKTLKNNVVEELFNLSDRLIDQEADIAQNNE |
| NO: 34) | SIEDLYDFGREVAESIGEIHAHNEAQNETLKDLITNSVKNTDNITKNKADIQALEN |
| | DVGKELLNLSGRLIDQKADIDNNINHIYELAQQQDQHSSDIKTLKNNVEEGLLELS |
| | GHLIDQKADLTKDIKALESNVEEGLLDLSGRLLDQKADIAQNQANIQDLAAYNEL |
| | QDAYAKQQTEAIDALNKASSENTQNIEDLAAYNELQDAYAKQQTEAIDALNKAS |
| | SENTQNIAKNQADIANNINNIYELAQQQDQHSSDIKTLAKASAANTDRIAKNKAD |
| | ADASFETLTKNQNTLIEKDKEHDKLITANKTAIDANKASADTKFAATADAITKNG |
| | NAITKNAKSITDLGTKVDGFDSRVTALDTKVNAFDGRITALDSKVENGMAAQAA |
| | LSGLFQPYSVGKFNATAALGGYGSKSAVAIGAGYRVNPNLAFKAGAAINTSGNK |
| | KGSYNIGVNYEF (616 amino acids) |
| Finnish | MKTMKLLPLKIAVTSAMIIGLGATSTVNAQVVEQFFPNIFFNENHDELDDAYHNM |
| FIN2344 | ILGDTAIVSNSQDNSTQLKFYSNDEDSVPDSLLFSKLLHEQQLNGFKAGDTIIPLDK |
| (SEQ ID | DGKPVYTKDTRTKDGKVETVYSVTTKIATQDDVEQSAYSRGIQGDIDDLYDINRE |
| NO: 35) | VNEYLKATHDYNERQTEAIDALNKASSANTDRIDTAEERIDKNEYDIKALESNVG |
| | KDLLDLSGRLIAQKEDIDNNINHIYELAQQQDQHSSDIKTLKNNVEEGLLELSGHLI |
| | DQKADLTKDIKTLESNVEEGLLDLSGRLIDQKADIAQNQANIQDLAAYNELQDQY |
| | AQKQTEAIDALNKASSENTQNIEDLAAYNELQDAYAKQQTEAIDALNKASSENTQ |
| | NIAKNQADIANNINNIYELAQQQDQHSSDIKTLAKVSAANTDRIAKNKADADASF |
| | ETLTKNQNTLIEKDKEHDKLITANKTAIDANKASADTKFAATADAITKNGNAITK |
| | NAKSITDLGTKVDGFDGRVTALDTKVNAFDGRITALDSKVENGMAAQAALSGLF |
| | QPYSVGKFNATAALGGYGSKSAVAIGAGYRVNPNLAFKAGAAINTSGNKKGSYN |
| | IGVNYEF (614 amino acids) |
| American | MKTMKLPPLKIAVTSAMIIGLGAASTANAQTTETFLPNLFDNDYTETTDPLYHGMI |
| V1118 | LGDTAITQDTQYKFYAENGNEVPDSLFFNKILHDQLLNGFKAGDTIIPLDENGKPV |
| (SEQ ID | YKLDERTENGVKRKVYSVTTKTATQADVEQSAYSRGIQGDIDDLYEANKENVNR |
| NO: 36) | LIEHGDKIFANEESVQYLNREVQNNIENIHELAQQQDQHSSDIKTLKKNVEKDLLD |
| | LSGRLIAQKEDIAQNQTDIQDLATYNELQDQYAQKQTEAIDALNKASSENTQNIA |
| | KNSNHIKTLENNIEECLLELSGHLIDQKADLTKDIKALESNVEEGLLDLSGRLIDQK |
| | ADIAQNQANIQDLAAYNELQDAYAKQQTEAIDALNKASSENTQNIEDLAAYNEL |
| | QDAYAKQQTEAIDALNKASSENTQNIEDLAAYNELQDAYAKQQTEAIDALNKAS |
| | SENTQNIAKNQADIANNINNIYELAQQQDQHSSDIKTLAKASAANTDRIAKNKAD |
| | ADASFETLTKNQNTLIEKDKEHDKLITANKTAIDANKASADTKFAATADAITKNG |
| | NAITKNAKSITDLGTKVDGFDGRVTALDTKVNALDTKVNAFDGRITALDSKVEN |
| | GMAAQAALSGLFQPYSVGKFNATAALGGYGSKSAVAIGAGYRVNPNLAFKAGA |
| | AINTSGNKKGSYNIGVNYEF (679 amino acids) |
| American | MKTMKLLPLKIAVTSALIVGLGAASTANAQETLEEVLESIKQINEQDLQDDIGYNS |
| V1145 | ALDRYLVLSQYGNLLIAKELNENVEKNSNSIAKNSNSIADLEADVGYLAENQNTLI |
| (SEQ ID | EQNETINQELEGITHELESFIAYAHAQDQKNLVNEFEIEKNKDAIAKNNESIEDLYD |
| NO: 37) | FGHEVAESIGEIHAYTEEVNKTLENLITNSVKNTDNITKNKADIQALESNVEKELL |
| | NLSGRLIDQKADIDNNINHIYELAQQQDQHSSDIKTLKKNVEEGLLELSGHLIDQK |
| | SDIAQNQTDIQDLATYNELQDQYAQKQTEAIDALNKASSENTQNIEDLAAYNELQ |
| | DAYAKQQTEAIDALNKASSENTQNIEDLAAYNELQDAYAKQQTEAIDALNKASS |
| | ENTQNIEDLAAYNELQDAYAKQQTEAIDALNKASSENTQNIEDLAAYNELQDAY |
| | AKQQTEAIDALNKASSENTQNIEDLAAYNELQDAYAKQQTEAIDALNKASSENTQ |
| | NIAKNQADIANNINNIYELAQQQDQHSSDIKTLAKASAANTDRIAKNKADADASF |
| | ETLTKNQNTLIEKDKEHDKLITANKTAIDANKASADTKFAATADAITKNGNAITK |
| | NAKSITDLGTKVDGFDSRVTALDTKVNAFDGRITALDSKVENGMAAQAALSGLF |
| | QPYSVGKFNATAALGGYGSKSAVAIGAGYRVNPNLAFKAGAAINTSGNKKGSYN |
| | IGVNYEF (724 amino acids) |
| American | MKTMKLLPLKIAVTSALIVGLGAASTANAQAQARDRSLEDIQALIGNIDVDKIRSQ |
| V1156 | KQKNPEIFQYLLLNQLSNTLITDELNNNVIKNTNSIETLDNDIAWLNDDLIDLDKEV |
| (SEQ ID | GVLSRDIGSLHDDVAQNQADIKTLENNVVEELFNLSDRLIDQEAEIAQNNESIEDL |
| NO: 38) | YDFGREVAESIGEIHAHNEAQNETLKDLITNSVKNTDNIDKNKADIQALENNVEEG |
| | LLELSGHLIDQKADLTKDIKALESNVEEGLLDLSGRLLDQKADIAKNQADIAQNQ |
| | TDIQDLAAYNELQDQYAQKQTEAIDALNKASSENTQNIEDLAAYNELQDAYAKQ |
| | QTEAIDALNKASSENTQNIEDLAAYNELQDAYAKQQTEAIDALNKASSENTQNIA |
| | KNQADIANNINNIYELAQQQDQHSSDIKTLAKVSAANTDRIAKNKADADASFETL |
| | TKNQNTLIEKDKEHDKLITANKTAIDANKASADTKFAATADAITKNGNAITKNAK |
| | SITDLGTKVDGFDSRVTALDTKVNAFDGRITALDSKVENGMAAQAALSGLFQPYS |
| | VGKFNATAALGGYGSKSAVAIGAGYRVNPNLAFKAGAAINTSGNKKGSYNIGVN |
| | YEF (611 amino acids) |

SEQ ID NO: 39-O46E uspA2H
MNKIYKVKKNAAGHSVACSEFAKGHTKKAVLGSLLIVGALGMATTASAQTGSTNAANG
NIISGVGAYVGGGVINQAKGNYPTVGGGFDNRATGNYSVISGGFDNQAKGEHSTIAGGES
NQATGRNSTVAGGSNNQAVGTNSTVAGGSNNQAKGANSFAAGVGNQANTDNAVALGK
NNTINGNNSAAIGSENTVNENQKNVFILGSNTTNAQSGSVLLGHETSGKEATAVSRARVN
GLTLKNFSGVSKADNGTVSVGSQGKERQIVHVGAGQISDDSTDAVNGSQLYALATAVDD
NQYDIEINQDNIKDLQKEVKGLDKEVGVLSRDIGSLHDDVADNQADIAKNKADIKELDKE
MNVLSRDIVSLNDDVADNQADIAKNQADIKTLENNVEEGLLDLSGRLIDQKADIDNNINHI
YELAQQQDQHSSDIKTLKKNVEEGLLELSGHLIDQKADIAQNQTDIQDLATYNELQDQYA
QKQTEAIDALNKASSENTQNIEDLAAYNELQDAYAKQQTEAIDALNKASSENTQNIEDLA
AYNELQDAYAKQQTEAIDALNKASSENTQNIEDLAAYNELQDAYAKQQTEAIDALNKAS
SENTQNIQDLAAYNELQDAYAKQQTEAIDALNKASSENTQNIQDLAAYNELQDAYAKQQ
TEAIDALNKASSENTQNIAKNQADIANNINNIYELAQQQDQHSSDIKTLAKASAANTDRIA
KNKADADASFETLTKNQNTLIEKDKEHDKLITANKTAIDANKASADTKFAATADAITKNG
NAITKNAKSITDLGTKVDGFDGRVTALDTKVNAFDGRITALDSKVENGMAAQAALSGLF
QPYSVGKFNATAALGGYGSKSAVAIGAGYRVNPNLAFKAGAAINTSGNKKGSYNIGVNY
EF
SEQ ID NO: 40-E22USPA2H
MNKIYKVKKNAAGHSVACSEFAKGHTKKAVLGSLLIVGALGMATTASAQLVSTTQPNDY
RSSTTGNNHLGSSWSIIGAGHDNIVYRSASNSGILSGYKNRVNGSTSAIVGGYDNETRGKY
TFVGGGYKNLAEGHQSAIGGGYANWAEGDNATIAGGFENFAAGNQSAIGGGYANLAEG
DDATIAGGFENRAEGNQSAIGGGYANFAAGDYTFVGGGYENRAEGNQSAIGGGYANLAE
GDNATIAGGFENRAKGINSVVSGGYANQATGESSTIAGGFENRAEGIDSVVSGGYANQAN
GAQSTVAGGYNNQATGESSTIAGGSNNQATGTGSFAAGVENKANADNAVALGKNNIING
DNSAAIGSNNTVKKGQKDVFILGSNTSGAQSNSVLLGNETTGKKATAVENATVGDLSLTG
FAGVSKANSGTVSVGSEGKERQIVHVGAGRISNDSTDAVNGSQLYALAAAVDDNQYDIE
KNQDDIKELKRGVKELDKEMNVLSRDIVSLNDDVAQNQSDIKTLKNNVEEGLLELSGHLI
DQKADLTKDIKALENNVEEGLLDLSGRLLDQKADIAKNQADIAQNQTDIQDLAAYNELQD
AYAKQQTEAIDALNKASSENTQNIEDLAAYNELQDAYAKQQTEAIDALNKASSENTQNIA
KNQADIANNINNIYELAQQQDQHSSDIKTLAKASAANTDRIAKNKADADASFETLTKNQN
TLIEKDKEHDKLITANKTAIDANKASADTKFAATADAITKNGNAITKNAKSITDLGTKVDG
FDGRVTALDTKVNAFDGRITALDSKVENGMAAQAALSGLFQPYSVGKFNATAALGGYGS
KSAVAIGAGYRVNPNLAFKAGAAINTSGNKKGSYNIGVNYEF
SEQ ID NO: 41-Mcat454epiUspA2H
VNKIYKVKKNVAGHSVACSEFAKGHTKKAVLGSLLIVGALGMATTASAQQNNKGFGAYI
GVENSGNLATGQYSVAVGGLGNEATGQYSVINGGYFNIADGVHSIIGGGKDNKAKGNYS
TIGGGDSNKAEGNYSTIAGGRNNEATGEGSFAAGIDNKANAKNAVALGNKNSIEGTNSVA
IGSNNTVAQDNTFILGSNTTGVQRNSVLLGNNTAGQEATIVEKAEVGGVNLTGFAGVSKT
SNGTVSVGSKNNERQIVNVGAGRISKDSTDAVNGSQLYALATAVDDSQYDIEKNQDDIEK
NQDDIKELKRGVKDLKKGVKGLDKEVKDLNKKVGVLDRDIGSLQDDVADNQADIANNQ
ADIKDLDKEVGVLSRDIGSLHDDVAQNQSDIKTLKNNVEEGLLELSGHLLDQKADIDNNIN
NIYELAQQQDQHSSDIKTLKKNVEEGLLELSGHLIDQKADIAQNQTDIQDLATYNELQDQY
AQKQTEAIDALNKASSENTQNIAKNSNRIKALESNVEEGLLELSGHLIDQKADLTKDIKAL
ESNVEEGLLDLSGRLIDQKADIAQNQANIQDLAAYNELQDQYAQKQTEAIDALNKASSEN
TQNIEDLAAYNELQDAYAKQQTEAIDALNKASSENTQNIAKNQADIANNINNIYELAQQQ
DQHSSDIKTLAKASAANTDRIAKNKADADASFETLTKNQNTLIEKDKEHDKLITANKTAID
ANKASADTKFAATADAITKNGNAITKNAKSITDLGTKVDGFDSRVTALDTKVNAFDGRIT
ALDSKVENGMAAQAALSGLFQPYSVGKFNATAALGGYGSKSAVAIGAGYRVNPNLAFK
AGAAINTSGNKKGSYNIGVNYEF
SEQ ID NO: 42-Mcat415epiUspA2H
VNKIYKVKKNVAGHSVACSEFAKGHTKKAVLGSLLIAGALGMATTASAQVVGNTTEGLG
ARVNGGDGNEAKGTYPTIGGGFFNIATGNYSTISGGNTNEAEGNYSTIGGGDTNAASGNN
STVVGGYNNTAKGNYSTVAGGANNQANSIDSTIVGGRKNRAKGKSSTVSGGYDNLAGG
KSSAIGGGEFNSAIGSRSTVAGGELNLASGDNATVSGGKQNQAIGEYSTVAGGELNLASG
DNATVSGGKQNQAEGTDSTIAGGRNNQAIGEGSFAAGIDNKANTENAVAVGKKNIINGDN
SAAIGSNNTVKKGQTGVFILGSNTKGAQSNSVLLGNETTGKKATAVSSATVNGLTLENFA
GVSQVGNGTVSVGSKGKERQIVNVGAGKISATSTDAVNGSQLHALATAVAYNYTDIIDNQ
ADIAKNQDDIDDLNKKVGVLDRDIGSLQDDVADNQADIANNQADIKDLDDEVGVLSRDIG
SLHDDVAQNQSDIKTLKNNVEEGLLDLSGRLLDQKADIDNNINNIYELAQQQDQHSSDIKT
LKKNVEEGLLELSGHLIDQKSDIAKNQADIAQNQTDIHDLAAYNELQDAYAKQQTEAIDA
LNKASSENTQNIEDLAAYNELQDAYAKQQTEAIDALNKASSENTQNIAKNQADIANNINNI
YELAQQQDQHSSDIKTLAKASAANTDRIAKNKADADASFETLTKNQNTLIEKDKEHDKLI
TANKTAIDANKASADTKFAATADAITKNGNAITKNAKSITDLGTKVDAFDGRVTALDTKV
NALDTKVNAFDGRITALDSKVENGMAAQAALSGLFQPYSVGKFNATAALGGYGSKSAVA
IGAGYRVNPNLAFKAGAAINTSGNKKGSYNIGVNYEF
SEQ ID NO: 43-RH4UspA2H
VNKIYKVKKNAAGHLVACSEFAKGHTKKAVLGSLLIVGILGMATTASAQDRAGNQRNGA
HSAIGGGSINIADGDYSTIGGGHINTTKGNYSTIGGGNENKTTGNYSTIGGGYGNEAEGKH
STIGGGDENKAKGDYSTIGGGDDNEATGTHSTIGGGDDNKATGDYSIIGGGYGNRATAED
ATVSGGGYNQAAGKSSTVSGGYDNLAEGESSAIGGGELNSTIGSHSTVAGGFLNGAVGDN
ATVSGGEQNQAIGKYSTVSGGYGNQATGIGSFAAGIENQANTENAVAVGKKNIINGDNSA
AIGSNNTVEKGQKDVFILGSNTKDAQSNSVLLGNETTGKAATVVDSANVGGLGLTGFAG
VSKVGNGTVSVGSQGKERQIVNVGAGEISATSTDAVNGSQLHALATAVDNNQYDIINNQG
DIERNQDDIKDLQKEVKGLDNEVGELSRDINSLHDVTDNQQDDIKELKRGVKELDKEMNV
LSRDIVSLNDDVADNQADIAKNQADIKTLESNVEEGLLDLSGRLLDQKADIANNVNHIYEL
AQQQDQHSSDIKTLKKNVEEGLLELSGHLIDQKSDIAQNQTDIQDLAAYNELQDQYAQKQ
TEAIDALNKASSENTQNIEDLAAYNELQDAYAKQQTEAIDALNKASSENTQNIAKNQADIA
NNINNIYELAQQQDQHSSDIKTLAKASAANTDRIAKNKADADASFETLTKNQNTLIEKDKE
HDKLITANKTAIDANKASADTKFAATADAITKNGNAITKNAKSITDLGTKVDGFDGRVTA
LDTKVNAFDGRITALDSKVENGMAAQAALSGLFQPYSVGKFNATAALGGYGSKSAVAIG
AGYRVNPNLAFKAGAAINTSGNKKGSYNIGVNYEF
SEQ ID NO: 44-Mcat031epiUspA2
MKTMKLLPLKIAVTSALIVGLGAASTANAQKKASSQELNQHIRVLNKYIRDFNDDIQELD
YDIQDLNDDIKGLDKKLSRDINSLRDEVTDNYTDIIDNQVDIERNQADIKDLKRGVKGLNK
EVGVLDRDIGSLQDDVADNQADIANNQDDIKDLDDEVGVLSRDIGSLHDDVAQNQSDIKT
LESNVEEGLLELSGHLIDQKADIDNNINHIYELAQQQDQHSSDIKTLKKNVEEGLLELSGHL
IDQKSDIAQNQTDIQDLATYNELQDQYAQKQTEAIDALNKASSENTQNIQDLAAYNELQD
AYAKQQTEAIDALNKASSENTQNIEDLAAYNELQDQYAQKQTEAIDALNKASSENTQNIQ
DLAAYNELQDAYAKQQTEAIDALNKASSENTQNIEDLAAYNELQDQYAQKQTEAIDALN
KASSENTQNIAKNQADIANNINNIYELAQQQDQHSSDIKTLAKASAANTDRIAKNKADAD
ASFETLTKNQNTLIEKDKEHDKLITANKTAIDANKASADTKFAATADAITKNGNAITKNAK
SITDLGTKVDGFDGRVTALDTKVNAFDGRITALDSKVENGMAAQAALSGLFQPYSVGKF
NATAALGGYGSKSAVAIGAGYRVNPNLAFKAGAAINTSGNKKGSYNIGVNYEF
SEQ ID NO: 45-MC2908uspA2
MKTMKLLPLKIAVTSALIVGLGAASTANAQLVERFFPNIFLDKPLAKQHYHNVVVGDTSIV
SDLQSNSDQLKFYSDDEGLVPDSLLFNKMLHEQLLNGFKEGDTIIPLDENGKPVYKVDYK
LDGKEPRKVYSVTTKIATAEDVATSSYANGIQKDIDDLYDFDHQVTERLTQHGKTIYRNG
ERILANEESVQYLNKEVQNNIEHIYELAQQQDQHSSDIKTLESNVEKGLLELSGHLIDQKA
DLTKDIKTLESNVEEGLLDLSGRLIDQKADLTKDIKTLESNVEEGLLDLSGRLIDQKADIAQ
NQANIQDLAAYNELQDQYAQKQTEAIDALNKASSENTQNIEDLAAYNELQDAYAKQQTE
AIDALNKASSENTQNIAKNQADIANNINNIYELAQQQDQHSSDIKTLAKASAANTNRIATA
ELGIAENKKDAQIAKAQANANKTAIDENKASADTKFAATADAITKNGNAITKNAKSITDL
GTKVDGFDSRVTALDTKVNAFDGRITALDSKVENGMAAQAALSGLFQPYSVGKFNATAA
LGGYGSKSAVAIGAGYRVNPNLAFKAGAAINTSGNKKGSYNIGVNYEF
SEQ ID NO: 46-Mox307uspa2
MKTMKLLPLKIAVTSAMIIGLGAASTANAQQQQQQQQQQQSRTEIFFPNIFFNENHDELDD
AYHNIILGDTALLDKQDGSQPQLKFYSNDKDSVPDSLLFSKLLHEQQLNGFKKGDTIIPLD
KDGKPVYQVDYKLDGKGKKQKRRQVYSVTTKTATDDDVNSAYSRGILGKVDDLDDEM
NFLNHDITSLYDVTANQQDAIKGLKKGVKGLNKELKELDKEVGVLSRDIGSLNDDVAQN
NESIEDLYDFSQEVADSIGEIHAHNKAQNETLQDLITNSVENTNNITKNKADIQALENNVVE
ELFNLSGRLIDQKADLTKDIKTLESNVEEGLLELSGHLIDQKADIAKNQADIAQNQANIQDL
AAYNELQDAYAKQQTEAIDALNKASSENTQNIEDLAAYNELQDAYAKQQTEAIDALNKA
SSENTQNIEDLAAYNELQDAYAKQQTEAIDALNKASSENTQNIAKNQADIANNINNIYELA
QQQDQHSSDIKTLAKASAANTDRIAKNKADADASFETLTKNQNTLIEKDKEHDKLITANK
TAIDENKASADTKFAATADAITKNGNAITKNAKSITDLGTKVDAFDGRVTALDTKVNAFD
GRITALDSKVENGMAAQAALSGLFQPYSVGKFNATAALGGYGSKSAVAIGAGYRVNPNL
AFKAGAAINTSGNKKGSYNIGVNYEF
SEQ ID NO: 47-UspA1 with signal peptide (Genbank Accession AAD43461.1)
MNKIYKVKKNAAGHLVACSEFAKGHTKKAVLGSLLIVGILGMATTASAQKVGKATNKIS
GGDNNTANGTYLTIGGGDYNKTKGRYSTIGGGLFNEATNEYSTIGSGGYNKAKGRYSTIG
GGGYNEATNQYSTIGGGDNNTAKGRYSTIGGGGYNEATIENSTVGGGGYNQAKGRNSTV
AGGYNNEATGTDSTIAGGRKNQATGKGSFAAGIDNKANADNAVALGNKNTIEGENSVAI
GSNNTVKKGQQNVFILGSNTDTTNAQNGSVLLGHNTAGKAATIVNSAEVGGLSLTGFAG
ASKTGNGTVSVGKKGKERQIVHVGAGEISDTSTDAVNGSQLHALATVVAQNKADIKDLD
DEVGLLGEEINSLEGEIFNNQDAIAKNQADIKTLESNVEEGLLDLSGRLLDQKADIDNNINN
IYELAQQQDQHSSDIKTLKNNVEEGLLDLSGRLIDQKADLTKDIKALESNVEEGLLDLSGR
LIDQKADIAKNQADIAQNQTDIQDLAAYNELQDAYAKQQTEAIDALNKASSANTDRIATA
ELGIAENKKDAQIAKAQANENKDGIAKNQADIQLHDKKITNLGILHSMVARAVGNNTQG
VATNKADIAKNQADIANNIKNIYELAQQQDQHSSDIKTLAKVSAANTDRIAKNKAEADAS
FETLTKNQNTLIEQGEALVEQNKAINQELEGFAAHADVQDKQILQNQADITTNKTAIEQNI
NRTVANGFEIEKNKAGIATNKQELILQNDRLNRINETNNHQDQKIDQLGYALKEQGQHFN
NRISAVERQTAGGIANAIAIATLPSPSRAGEHHVLFGSGYHNGQAAVSLGAAGLSDTGKST
YKIGLSWSDAGGLSGGVGGSYRWK
SEQ ID NO: 48-UspA1 without signal peptide (Genbank Accession AAD43461.1)
QKVGKATNKISGGDNNTANGTYLTIGGGDYNKTKGRYSTIGGGLFNEATNEYSTIGSGGY
NKAKGRYSTIGGGGYNEATNQYSTIGGGDNNTAKGRYSTIGGGGYNEATIENSTVGGGGY
NQAKGRNSTVAGGYNNEATGTDSTIAGGRKNQATGKGSFAAGIDNKANADNAVALGNK
NTIEGENSVAIGSNNTVKKGQQNVFILGSNTDTTNAQNGSVLLGHNTAGKAATIVNSAEV
GGLSLTGFAGASKTGNGTVSVGKKGKERQIVHVGAGEISDTSTDAVNGSQLHALATVVA
QNKADIKDLDDEVGLLGEEINSLEGEIFNNQDAIAKNQADIKTLESNVEEGLLDLSGRLLD
QKADIDNNINNIYELAQQQDQHSSDIKTLKNNVEEGLLDLSGRLIDQKADLTKDIKALESN
VEEGLLDLSGRLIDQKADIAKNQADIAQNQTDIQDLAAYNELQDAYAKQQTEAIDALNKA
SSANTDRIATAELGIAENKKDAQIAKAQANENKDGIAKNQADIQLHDKKITNLGILHSMVA
RAVGNNTQGVATNKADIAKNQADIANNIKNIYELAQQQDQHSSDIKTLAKVSAANTDRIA
KNKAEADASFETLTKNQNTLIEQGEALVEQNKAINQELEGFAAHADVQDKQILQNQADIT
TNKTAIEQNINRTVANGFEIEKNKAGIATNKQELILQNDRLNRINETNNHQDQKIDQLGYA
LKEQGQHFNNRISAVERQTAGGIANAIAIATLPSPSRAGEHHVLFGSGYHNGQAAVSLGAA
GLSDTGKSTYKIGLSWSDAGGLSGGVGGSYRWK
SEQ ID NO: 49-UspA2H with signal peptide from Strain RH4 (GenBank:
AGH27471.1)
MNKIYKVKKNAAGHLVACSEFAKGHTKKAVLGSLLIVGILGMATTASAQDRAGNQRNG
AHSAIGGGSINIADGDYSTIGGGHINTTKGNYSTIGGGNENKTTGNYSTIGGGYGNEAEGK
HSTIGGGDENKAKGDYSTIGGGDDNEATGTHSTIGGGDDNKATGDYSIIGGGYGNRATAE
DATVSGGGYNQAAGKSSTVSGGYDNLAEGESSAIGGGELNSTIGSHSTVAGGFLNGAVGD
NATVSGGEQNQAIGKYSTVSGGYGNQATGIGSFAAGIENQANTENAVAVGKKNIINGDNS
AAIGSNNTVEKGQKDVFILGSNTKDAQSNSVLLGNETTGKAATVVDSANVGGLGLTGFA
GVSKVGNGTVSVGSQGKERQIVNVGAGEISATSTDAVNGSQLHALATAVDNNQYDIINNQ
GDIERNQDDIKDLQKEVKGLDNEVGELSRDINSLHDVTDNQQDDIKELKRGVKELDKEMN
VLSRDIVSLNDDVADNQADIAKNQADIKTLESNVEEGLLDLSGRLLDQKADIANNVNHIYE
LAQQQDQHSSDIKTLKKNVEEGLLELSGHLIDQKSDIAQNQTDIQDLAAYNELQDQYAQK
QTEAIDALNKASSENTQNIEDLAAYNELQDAYAKQQTEAIDALNKASSENTQNIAKNQADI
ANNINNIYELAQQQDQHSSDIKTLAKASAANTDRIAKNKADADASFETLTKNQNTLIEKDK
EHDKLITANKTAIDANKASADTKFAATADAITKNGNAITKNAKSITDLGTKVDGFDGRVT
ALDTKVNAFDGRITALDSKVENGMAAQAALSGLFQPYSVGKFNATAALGGYGSKSAVAI
GAGYRVNPNLAFKAGAAINTSGNKKGSYNIGVNYEF
SEQ ID NO: 50-UspA2H without signal peptide from Strain RH4 (GenBank:
AGH27471.1)
QDRAGNQRNGAHSAIGGGSINIADGDYSTIGGGHINTTKGNYSTIGGGNENKTTGNYSTIG
GGYGNEAEGKHSTIGGGDENKAKGDYSTIGGGDDNEATGTHSTIGGGDDNKATGDYSIIG
GGYGNRATAEDATVSGGGYNQAAGKSSTVSGGYDNLAEGESSAIGGGELNSTIGSHSTVA
GGFLNGAVGDNATVSGGEQNQAIGKYSTVSGGYGNQATGIGSFAAGIENQANTENAVAV
GKKNIINGDNSAAIGSNNTVEKGQKDVFILGSNTKDAQSNSVLLGNETTGKAATVVDSAN
VGGLGLTGFAGVSKVGNGTVSVGSQGKERQIVNVGAGEISATSTDAVNGSQLHALATAV
DNNQYDIINNQGDIERNQDDIKDLQKEVKGLDNEVGELSRDINSLHDVTDNQQDDIKELK
RGVKELDKEMNVLSRDIVSLNDDVADNQADIAKNQADIKTLESNVEEGLLDLSGRLLDQK
ADIANNVNHIYELAQQQDQHSSDIKTLKKNVEEGLLELSGHLIDQKSDIAQNQTDIQDLAA
YNELQDQYAQKQTEAIDALNKASSENTQNIEDLAAYNELQDAYAKQQTEAIDALNKASSE
NTQNIAKNQADIANNINNIYELAQQQDQHSSDIKTLAKASAANTDRIAKNKADADASFETL
TKNQNTLIEKDKEHDKLITANKTAIDANKASADTKFAATADAITKNGNAITKNAKSITDLG
TKVDGFDGRVTALDTKVNAFDGRITALDSKVENGMAAQAALSGLFQPYSVGKFNATAAL
GGYGSKSAVAIGAGYRVNPNLAFKAGAAINTSGNKKGSYNIGVNYEF
SEQ ID NO: 51-UspA2V with signal peptide from Strain S43:4 (GenBank:
AEQ18817.1)
MKTMKLLPLKIAVTSAMIVGLGAASNANAQTSNSGTNQIVEEFLPKLTQALNDPQYQHEF
EDALDQYHNMVVGNTSFVSTVDVANQTVGNTALKFYSDSENTLPSSMLFDQILTNQRLN
GFKEGQPFIPVDDNGNPITQVDDVLESNGQTKKVRSVTTRIATAEDVTKSPYAQGIQKDID
DIYEFNHEVVDNIATLNRATEILTEAGNHHDELIRKNKADIQVSNGKIESLKTLYETTVRTV
GNNESNIKTNKDAIAKNKANIKTLAQAQLESVTFLGEQIQENKDDISAVTGKAFKNQIDIQ
RNNDSIEDLYEANNDNVERLVEHDRAIEILAEAGNHHDTLIRKNKADIQDLAAHNELQTE
AIDALNKASSANTKRITTAELGIAENKKDALIAKAQADKNKNDIQDLAKVQRAGVEVMAE
LNKNIAGTQTDIANNKITLAQHAKKIDENQQAIEAKLGGKADLQKLSELKTSVDKNQAGI
QLHDQKINNLGILHTMVARAVGANKTAIDQNKADIAENKEDIQNNINNIYELAQQQDQHS
SDIKTLAKASSANTDNIAKNKADADASFKTLTKNQNTLIEKDKAQDALITANKADADASF
KTLTKNQNTLIEKDKAQDKLITANKADADAKFTGVQTDIANNKTTLAQHAKKINDNQQAI
EAKLGGKADAQALNNLNDKVNGFDGRISALDTKVNAFDGRITALDSKVENGMAAQAAL
SGLFQPYSVGKFNATAALGGYGSKSAVAIGAGYRVNPNLAIKAGAAINTSGDKKGSYNIG
VNYEF
SEQ ID NO: 52-UspA2V without signal peptide from Strain S43:4 (GenBank:
AEQ18817.1)
QTSNSGTNQIVEEFLPKLTQALNDPQYQHEFEDALDQYHNMVVGNTSFVSTVDVANQTV
GNTALKFYSDSENTLPSSMLFDQILTNQRLNGFKEGQPFIPVDDNGNPITQVDDVLESNGQ
TKKVRSVTTRIATAEDVTKSPYAQGIQKDIDDIYEFNHEVVDNIATLNRATEILTEAGNHH
DELIRKNKADIQVSNGKIESLKTLYETTVRTVGNNESNIKTNKDAIAKNKANIKTLAQAQL
ESVTFLGEQIQENKDDISAVTGKAFKNQIDIQRNNDSIEDLYEANNDNVERLVEHDRAIEIL
AEAGNHHDTLIRKNKADIQDLAAHNELQTEAIDALNKASSANTKRITTAELGIAENKKDAL
IAKAQADKNKNDIQDLAKVQRAGVEVMAELNKNIAGTQTDIANNKITLAQHAKKIDENQ
QAIEAKLGGKADLQKLSELKTSVDKNQAGIQLHDQKINNLGILHTMVARAVGANKTAIDQ
NKADIAENKEDIQNNINNIYELAQQQDQHSSDIKTLAKASSANTDNIAKNKADADASFKTL
TKNQNTLIEKDKAQDALITANKADADASFKTLTKNQNTLIEKDKAQDKLITANKADADAK
FTGVQTDIANNKTTLAQHAKKINDNQQAIEAKLGGKADAQALNNLNDKVNGFDGRISAL
DTKVNAFDGRITALDSKVENGMAAQAALSGLFQPYSVGKFNATAALGGYGSKSAVAIGA
GYRVNPNLAIKAGAAINTSGDKKGSYNIGVNYEF
SEQ ID NO: 53-Amino acids 30-540 of UspA2 from SEQ ID NO: 1
QAKNDITLEDLPYLIKKIDQNELEADIGDITALEKYLALSQYGNILALEELNKALEELDEDV
GWNQNDIANLEDDVETLTKNQNALAEQGEAIKEDLQGLADFVEGQEGKILQNETSIKKNT
QRNLVNGFEIEKNKDAIAKNNESIEDLYDFGHEVAESIGEIHAHNEAQNETLKGLITNSIEN
TNNITKNKADIQALENNVVEELFNLSGRLIDQKADIDNNINNIYELAQQQDQHSSDIKTLKK
NVEEGLLELSGHLIDQKTDIAQNQANIQDLATYNELQDQYAQKQTEAIDALNKASSENTQ
NIEDLAAYNELQDAYAKQQTEAIDALNKASSENTQNIEDLAAYNELQDAYAKQQTEAIDA
LNKASSENTQNIAKNQADIANNINNIYELAQQQDQHSSDIKTLAKASAANTDRIAKNKAD
ADASFETLTKNQNTLIEKDKEHDKLITANKTAIDANKASADTKFAATADAITKNGNAITKN
AKSITDLGTKVDGFDSRVTALDTK
SEQ ID NO: 54-Amino acids 31-540 of UspA2 from SEQ ID NO: 1
AKNDITLEDLPYLIKKIDQNELEADIGDITALEKYLALSQYGNILALEELNKALEELDEDVG
WNQNDIANLEDDVETLTKNQNALAEQGEAIKEDLQGLADFVEGQEGKILQNETSIKKNTQ
RNLVNGFEIEKNKDAIAKNNESIEDLYDFGHEVAESIGEIHAHNEAQNETLKGLITNSIENT
NNITKNKADIQALENNVVEELFNLSGRLIDQKADIDNNINNIYELAQQQDQHSSDIKTLKK
NVEEGLLELSGHLIDQKTDIAQNQANIQDLATYNELQDQYAQKQTEAIDALNKASSENTQ
NIEDLAAYNELQDAYAKQQTEAIDALNKASSENTQNIEDLAAYNELQDAYAKQQTEAIDA
LNKASSENTQNIAKNQADIANNINNIYELAQQQDQHSSDIKTLAKASAANTDRIAKNKAD
ADASFETLTKNQNTLIEKDKEHDKLITANKTAIDANKASADTKFAATADAITKNGNAITKN
AKSITDLGTKVDGFDSRVTALDTK
SEQ ID NO: 55: Amino acids 30-519 of UspA2 from SEQ ID NO: 1
QAKNDITLEDLPYLIKKIDQNELEADIGDITALEKYLALSQYGNILALEELNKALEELDEDV
GWNQNDIANLEDDVETLTKNQNALAEQGEAIKEDLQGLADFVEGQEGKILQNETSIKKNT
QRNLVNGFEIEKNKDAIAKNNESIEDLYDFGHEVAESIGEIHAHNEAQNETLKGLITNSIEN
TNNITKNKADIQALENNVVEELFNLSGRLIDQKADIDNNINNIYELAQQQDQHSSDIKTLKK
NVEEGLLELSGHLIDQKTDIAQNQANIQDLATYNELQDQYAQKQTEAIDALNKASSENTQ
NIEDLAAYNELQDAYAKQQTEAIDALNKASSENTQNIEDLAAYNELQDAYAKQQTEAIDA
LNKASSENTQNIAKNQADIANNINNIYELAQQQDQHSSDIKTLAKASAANTDRIAKNKAD
ADASFETLTKNQNTLIEKDKEHDKLITANKTAIDANKASADTKFAATADAITKNGNAITKN
AKS
SEQ ID NO: 56: Amino acids 30-564 of UspA2 from SEQ ID NO: 1
QAKNDITLEDLPYLIKKIDQNELEADIGDITALEKYLALSQYGNILALEELNKALEELDEDV
GWNQNDIANLEDDVETLTKNQNALAEQGEAIKEDLQGLADFVEGQEGKILQNETSIKKNT
QRNLVNGFEIEKNKDAIAKNNESIEDLYDFGHEVAESIGEIHAHNEAQNETLKGLITNSIEN
TNNITKNKADIQALENNVVEELFNLSGRLIDQKADIDNNINNIYELAQQQDQHSSDIKTLKK
NVEEGLLELSGHLIDQKTDIAQNQANIQDLATYNELQDQYAQKQTEAIDALNKASSENTQ
NIEDLAAYNELQDAYAKQQTEAIDALNKASSENTQNIEDLAAYNELQDAYAKQQTEAIDA
LNKASSENTQNIAKNQADIANNINNIYELAQQQDQHSSDIKTLAKASAANTDRIAKNKAD
ADASFETLTKNQNTLIEKDKEHDKLITANKTAIDANKASADTKFAATADAITKNGNAITKN
AKSITDLGTKVDGFDSRVTALDTKVNAFDGRITALDSKVENGMAAQAA
SEQ ID NO: 57: Amino acids 31-564 of UspA2 from SEQ ID NO: 1
AKNDITLEDLPYLIKKIDQNELEADIGDITALEKYLALSQYGNILALEELNKALEELDEDVG
WNQNDIANLEDDVETLTKNQNALAEQGEAIKEDLQGLADFVEGQEGKILQNETSIKKNTQ
RNLVNGFEIEKNKDAIAKNNESIEDLYDFGHEVAESIGEIHAHNEAQNETLKGLITNSIENT
NNITKNKADIQALENNVVEELFNLSGRLIDQKADIDNNINNIYELAQQQDQHSSDIKTLKK
NVEEGLLELSGHLIDQKTDIAQNQANIQDLATYNELQDQYAQKQTEAIDALNKASSENTQ
NIEDLAAYNELQDAYAKQQTEAIDALNKASSENTQNIEDLAAYNELQDAYAKQQTEAIDA
LNKASSENTQNIAKNQADIANNINNIYELAQQQDQHSSDIKTLAKASAANTDRIAKNKAD
ADASFETLTKNQNTLIEKDKEHDKLITANKTAIDANKASADTKFAATADAITKNGNAITKN
AKSITDLGTKVDGFDSRVTALDTKVNAFDGRITALDSKVENGMAAQAA
MC-001 (DNA)-SEQ ID NO: 58
ATGCAGGCCAAAAATGATATTACCCTGGAAGATCTGCCGTATCTGATCAAAAAAATCG
ATCAGAACGAACTGGAAGCCGATATTGGTGATATTACCGCACTGGAAAAATATCTGGC
ACTGAGCCAGTATGGAAATATTCTGGCCCTGGAAGAACTGAATAAAGCTCTGGAAGA
GCTGGATGAAGATGTGGGTTGGAATCAGAATGATATCGCCAATCTGGAAGATGATGTT
GAAACCCTGACCAAAAATCAGAATGCACTGGCAGAACAGGGTGAAGCAATTAAAGAA
GATCTGCAGGGTCTGGCAGATTTTGTTGAAGGTCAGGAAGGCAAAATTCTGCAGAACG
AAACCAGCATCAAAAAAAACACCCAGCGTAATCTGGTGAATGGCTTTGAAATTGAAA
AAAACAAAGATGCCATTGCCAAAAACAACGAAAGCATTGAAGATCTGTATGATTTTG
GTCATGAAGTTGCCGAAAGCATTGGTGAAATTCATGCACATAACGAAGCACAGAATG
AAACCCTGAAAGGTCTGATTACCAACAGCATCGAAAATACCAATAACATTACCAAAA
ACAAAGCAGATATTCAGGCGCTGGAAAATAATGTTGTGGAAGAACTGTTTAATCTGAG
CGGTCGTCTGATTGATCAGAAAGCCGATATCGATAATAACATTAACAACATTTATGAA
CTGGCACAGCAGCAGGATCAGCATAGCAGCGATATCAAAACCCTGAAAAAAAACGTT
GAAGAAGGTCTGCTGGAACTGTCTGGTCACCTGATCGATCAGAAAACTGATATTGCCC
AGAATCAGGCAAATATTCAGGATCTGGCCACCTATAATGAACTGCAGGATCAGTATGC
ACAGAAACAGACCGAAGCAATTGATGCCCTGAATAAAGCGAGCAGCGAAAACACCCA
GAATATCGAAGATCTGGCAGCATACAACGAACTGCAGGATGCCTATGCAAAACAGCA
GACTGAAGCCATCGACGCACTGAACAAGGCAAGCTCTGAAAACACGCAGAACATTGA
AGATCTGGCTGCCTATAATGAATTACAGGATGCGTATGCCAAACAGCAGACCGAAGC
GATTGATGCGCTGAACAAAGCCTCTTCTGAAAATACACAGAATATCGCCAAAAATCAG
GCCGATATTGCCAACAATATCAATAATATCTATGAACTGGCCCAGCAGCAGGATCAGC
ACTCTTCTGATATCAAAACACTGGCAAAAGCAAGCGCAGCAAATACCGATCGTATTGC
GAAAAACAAAGCCGATGCAGATGCAAGCTTTGAAACACTGACGAAAAACCAGAACAC
CCTGATTGAAAAAGATAAAGAACATGATAAACTGATCACCGCCAATAAAACCGCAAT
TGATGCAAATAAAGCCAGCGCAGATACCAAATTTGCAGCAACCGCAGATGCAATTAC
CAAAAATGGCAATGCCATCACCAAAAATGCCAAAAGCATTACCGATCTGGGCACCAA
AGTTGATGGTTTTGATAGCCGTGTGACCGCACTGGATACCAAAGCAAGCCATCATCAT
CACCACCACTAA
MC-001 (protein)-(M)(UspA2 amino acids 30-540)(ASHHHHHH) SEQ ID NO: 59
MQAKNDITLEDLPYLIKKIDQNELEADIGDITALEKYLALSQYGNILALEELNKALEELDED
VGWNQNDIANLEDDVETLTKNQNALAEQGEAIKEDLQGLADFVEGQEGKILQNETSIKKN
TQRNLVNGFEIEKNKDAIAKNNESIEDLYDFGHEVAESIGEIHAHNEAQNETLKGLITNSIE
NTNNITKNKADIQALENNVVEELFNLSGRLIDQKADIDNNINNIYELAQQQDQHSSDIKTLK
KNVEEGLLELSGHLIDQKTDIAQNQANIQDLATYNELQDQYAQKQTEAIDALNKASSENT
QNIEDLAAYNELQDAYAKQQTEAIDALNKASSENTQNIEDLAAYNELQDAYAKQQTEAID
ALNKASSENTQNIAKNQADIANNINNIYELAQQQDQHSSDIKTLAKASAANTDRIAKNKA
DADASFETLTKNQNTLIEKDKEHDKLITANKTAIDANKASADTKFAATADAITKNGNAITK
NAKSITDLGTKVDGFDSRVTALDTKASHHHHHH
MC-002 (DNA)-SEQ ID NO: 60
ATGCAGGCCAAAAATGATATTACCCTGGAAGATCTGCCGTATCTGATCAAAAAAATCG
ATCAGAACGAACTGGAAGCCGATATTGGTGATATTACCGCACTGGAAAAATATCTGGC
ACTGAGCCAGTATGGAAATATTCTGGCCCTGGAAGAACTGAATAAAGCTCTGGAAGA
GCTGGATGAAGATGTGGGTTGGAATCAGAATGATATCGCCAATCTGGAAGATGATGTT
GAAACCCTGACCAAAAATCAGAATGCACTGGCAGAACAGGGTGAAGCAATTAAAGAA
GATCTGCAGGGTCTGGCAGATTTTGTTGAAGGTCAGGAAGGCAAAATTCTGCAGAACG
AAACCAGCATCAAAAAAAACACCCAGCGTAATCTGGTGAATGGCTTTGAAATTGAAA
AAAACAAAGATGCCATTGCCAAAAACAACGAAAGCATTGAAGATCTGTATGATTTTG
GTCATGAAGTTGCCGAAAGCATTGGTGAAATTCATGCACATAACGAAGCACAGAATG
AAACCCTGAAAGGTCTGATTACCAACAGCATCGAAAATACCAATAACATTACCAAAA
ACAAAGCAGATATTCAGGCGCTGGAAAATAATGTTGTGGAAGAACTGTTTAATCTGAG
CGGTCGTCTGATTGATCAGAAAGCCGATATCGATAATAACATTAACAACATTTATGAA
CTGGCACAGCAGCAGGATCAGCATAGCAGCGATATCAAAACCCTGAAAAAAAACGTT
GAAGAAGGTCTGCTGGAACTGTCTGGTCACCTGATCGATCAGAAAACTGATATTGCCC
AGAATCAGGCAAATATTCAGGATCTGGCCACCTATAATGAACTGCAGGATCAGTATGC
ACAGAAACAGACCGAAGCAATTGATGCCCTGAATAAAGCGAGCAGCGAAAACACCCA
GAATATCGAAGATCTGGCAGCATACAACGAACTGCAGGATGCCTATGCAAAACAGCA
GACTGAAGCCATCGACGCACTGAACAAGGCAAGCTCTGAAAACACGCAGAACATTGA
AGATCTGGCTGCCTATAATGAATTACAGGATGCGTATGCCAAACAGCAGACCGAAGC
GATTGATGCGCTGAACAAAGCCTCTTCTGAAAATACACAGAATATCGCCAAAAATCAG
GCCGATATTGCCAACAATATCAATAATATCTATGAACTGGCCCAGCAGCAGGATCAGC
ACTCTTCTGATATCAAAACACTGGCAAAAGCAAGCGCAGCAAATACCGATCGTATTGC
GAAAAACAAAGCCGATGCAGATGCAAGCTTTGAAACACTGACGAAAAACCAGAACAC
CCTGATTGAAAAAGATAAAGAACATGATAAACTGATCACCGCCAATAAAACCGCAAT
TGATGCAAATAAAGCCAGCGCAGATACCAAATTTGCAGCAACCGCAGATGCAATTAC
CAAAAATGGCAATGCCATCACCAAAAATGCCAAAAGCATTACCGATCTGGGCACCAA
AGTTGATGGTTTTGATAGCCGTGTGACCGCACTGGATACCAAATAA
MC-002 (Protein)-(M)(UspA2 amino acids 30-540) SEQ ID NO: 61
MQAKNDITLEDLPYLIKKIDQNELEADIGDITALEKYLALSQYGNILALEELNKALEELDED
VGWNQNDIANLEDDVETLTKNQNALAEQGEAIKEDLQGLADFVEGQEGKILQNETSIKKN
TQRNLVNGFEIEKNKDAIAKNNESIEDLYDFGHEVAESIGEIHAHNEAQNETLKGLITNSIE
NTNNITKNKADIQALENNVVEELFNLSGRLIDQKADIDNNINNIYELAQQQDQHSSDIKTLK
KNVEEGLLELSGHLIDQKTDIAQNQANIQDLATYNELQDQYAQKQTEAIDALNKASSENT
QNIEDLAAYNELQDAYAKQQTEAIDALNKASSENTQNIEDLAAYNELQDAYAKQQTEAID
ALNKASSENTQNIAKNQADIANNINNIYELAQQQDQHSSDIKTLAKASAANTDRIAKNKA
DADASFETLTKNQNTLIEKDKEHDKLITANKTAIDANKASADTKFAATADAITKNGNAITK
NAKSITDLGTKVDGFDSRVTALDTK
MC-003 (DNA)-SEQ ID NO: 62
ATGCAGGCCAAAAATGATATTACCCTGGAAGATCTGCCGTATCTGATCAAAAAAATCG
ATCAGAACGAACTGGAAGCCGATATTGGTGATATTACCGCACTGGAAAAATATCTGGC
ACTGAGCCAGTATGGAAATATTCTGGCCCTGGAAGAACTGAATAAAGCTCTGGAAGA
GCTGGATGAAGATGTGGGTTGGAATCAGAATGATATCGCCAATCTGGAAGATGATGTT
GAAACCCTGACCAAAAATCAGAATGCACTGGCAGAACAGGGTGAAGCAATTAAAGAA
GATCTGCAGGGTCTGGCAGATTTTGTTGAAGGTCAGGAAGGCAAAATTCTGCAGAACG
AAACCAGCATCAAAAAAAACACCCAGCGTAATCTGGTGAATGGCTTTGAAATTGAAA
AAAACAAAGATGCCATTGCCAAAAACAACGAAAGCATTGAAGATCTGTATGATTTTG
GTCATGAAGTTGCCGAAAGCATTGGTGAAATTCATGCACATAACGAAGCACAGAATG
AAACCCTGAAAGGTCTGATTACCAACAGCATCGAAAATACCAATAACATTACCAAAA
ACAAAGCAGATATTCAGGCGCTGGAAAATAATGTTGTGGAAGAACTGTTTAATCTGAG
CGGTCGTCTGATTGATCAGAAAGCCGATATCGATAATAACATTAACAACATTTATGAA
CTGGCACAGCAGCAGGATCAGCATAGCAGCGATATCAAAACCCTGAAAAAAAACGTT
GAAGAAGGTCTGCTGGAACTGTCTGGTCACCTGATCGATCAGAAAACTGATATTGCCC
AGAATCAGGCAAATATTCAGGATCTGGCCACCTATAATGAACTGCAGGATCAGTATGC
ACAGAAACAGACCGAAGCAATTGATGCCCTGAATAAAGCGAGCAGCGAAAACACCCA
GAATATCGAAGATCTGGCAGCATACAACGAACTGCAGGATGCCTATGCAAAACAGCA
GACTGAAGCCATCGACGCACTGAACAAGGCAAGCTCTGAAAACACGCAGAACATTGA
AGATCTGGCTGCCTATAATGAATTACAGGATGCGTATGCCAAACAGCAGACCGAAGC
GATTGATGCGCTGAACAAAGCCTCTTCTGAAAATACACAGAATATCGCCAAAAATCAG
GCCGATATTGCCAACAATATCAATAATATCTATGAACTGGCCCAGCAGCAGGATCAGC
ACTCTTCTGATATCAAAACACTGGCAAAAGCAAGCGCAGCAAATACCGATCGTATTGC
GAAAAACAAAGCCGATGCAGATGCAAGCTTTGAAACACTGACGAAAAACCAGAACAC
CCTGATTGAAAAAGATAAAGAACATGATAAACTGATCACCGCCAATAAAACCGCAAT
TGATGCAAATAAAGCCAGCGCAGATACCAAATTTGCAGCAACCGCAGATGCAATTAC
CAAAAATGGCAATGCCATCACCAAAAATGCCAAAAGCATTACCGATCTGGGCACCAA
AGTTGATGGTTTTGATAGCCGTGTGACCGCACTGGATACCAAACACTAA
MC-003 (Protein)-(M)(UspA2 amino acids 30-540)(H)-SEQ ID NO: 63
MQAKNDITLEDLPYLIKKIDQNELEADIGDITALEKYLALSQYGNILALEELNKALEELDED
VGWNQNDIANLEDDVETLTKNQNALAEQGEAIKEDLQGLADFVEGQEGKILQNETSIKKN
TQRNLVNGFEIEKNKDAIAKNNESIEDLYDFGHEVAESIGEIHAHNEAQNETLKGLITNSIE
NTNNITKNKADIQALENNVVEELFNLSGRLIDQKADIDNNINNIYELAQQQDQHSSDIKTLK
KNVEEGLLELSGHLIDQKTDIAQNQANIQDLATYNELQDQYAQKQTEAIDALNKASSENT
QNIEDLAAYNELQDAYAKQQTEAIDALNKASSENTQNIEDLAAYNELQDAYAKQQTEAID
ALNKASSENTQNIAKNQADIANNINNIYELAQQQDQHSSDIKTLAKASAANTDRIAKNKA
DADASFETLTKNQNTLIEKDKEHDKLITANKTAIDANKASADTKFAATADAITKNGNAITK
NAKSITDLGTKVDGFDSRVTALDTKH
MC-004 (DNA)-SEQ ID NO: 64
ATGCAGGCCAAAAATGATATTACCCTGGAAGATCTGCCGTATCTGATCAAAAAAATCG
ATCAGAACGAACTGGAAGCCGATATTGGTGATATTACCGCACTGGAAAAATATCTGGC
ACTGAGCCAGTATGGAAATATTCTGGCCCTGGAAGAACTGAATAAAGCTCTGGAAGA
GCTGGATGAAGATGTGGGTTGGAATCAGAATGATATCGCCAATCTGGAAGATGATGTT
GAAACCCTGACCAAAAATCAGAATGCACTGGCAGAACAGGGTGAAGCAATTAAAGAA
GATCTGCAGGGTCTGGCAGATTTTGTTGAAGGTCAGGAAGGCAAAATTCTGCAGAACG
AAACCAGCATCAAAAAAAACACCCAGCGTAATCTGGTGAATGGCTTTGAAATTGAAA
AAAACAAAGATGCCATTGCCAAAAACAACGAAAGCATTGAAGATCTGTATGATTTTG
GTCATGAAGTTGCCGAAAGCATTGGTGAAATTCATGCACATAACGAAGCACAGAATG
AAACCCTGAAAGGTCTGATTACCAACAGCATCGAAAATACCAATAACATTACCAAAA
ACAAAGCAGATATTCAGGCGCTGGAAAATAATGTTGTGGAAGAACTGTTTAATCTGAG
CGGTCGTCTGATTGATCAGAAAGCCGATATCGATAATAACATTAACAACATTTATGAA
CTGGCACAGCAGCAGGATCAGCATAGCAGCGATATCAAAACCCTGAAAAAAAACGTT
GAAGAAGGTCTGCTGGAACTGTCTGGTCACCTGATCGATCAGAAAACTGATATTGCCC
AGAATCAGGCAAATATTCAGGATCTGGCCACCTATAATGAACTGCAGGATCAGTATGC
ACAGAAACAGACCGAAGCAATTGATGCCCTGAATAAAGCGAGCAGCGAAAACACCCA
GAATATCGAAGATCTGGCAGCATACAACGAACTGCAGGATGCCTATGCAAAACAGCA
GACTGAAGCCATCGACGCACTGAACAAGGCAAGCTCTGAAAACACGCAGAACATTGA
AGATCTGGCTGCCTATAATGAATTACAGGATGCGTATGCCAAACAGCAGACCGAAGC
GATTGATGCGCTGAACAAAGCCTCTTCTGAAAATACACAGAATATCGCCAAAAATCAG
GCCGATATTGCCAACAATATCAATAATATCTATGAACTGGCCCAGCAGCAGGATCAGC
ACTCTTCTGATATCAAAACACTGGCAAAAGCAAGCGCAGCAAATACCGATCGTATTGC
GAAAAACAAAGCCGATGCAGATGCAAGCTTTGAAACACTGACGAAAAACCAGAACAC
CCTGATTGAAAAAGATAAAGAACATGATAAACTGATCACCGCCAATAAAACCGCAAT
TGATGCAAATAAAGCCAGCGCAGATACCAAATTTGCAGCAACCGCAGATGCAATTAC
CAAAAATGGCAATGCCATCACCAAAAATGCCAAAAGCATTACCGATCTGGGCACCAA
AGTTGATGGTTTTGATAGCCGTGTGACCGCACTGGATACCAAACATCATTAA
MC-004 (Protein)-(M)(UspA2 amino acids 30-540)(HH) SEQ ID NO: 65
MQAKNDITLEDLPYLIKKIDQNELEADIGDITALEKYLALSQYGNILALEELNKALEELDED
VGWNQNDIANLEDDVETLTKNQNALAEQGEAIKEDLQGLADFVEGQEGKILQNETSIKKN
TQRNLVNGFEIEKNKDAIAKNNESIEDLYDFGHEVAESIGEIHAHNEAQNETLKGLITNSIE
NTNNITKNKADIQALENNVVEELFNLSGRLIDQKADIDNNINNIYELAQQQDQHSSDIKTLK
KNVEEGLLELSGHLIDQKTDIAQNQANIQDLATYNELQDQYAQKQTEAIDALNKASSENT
QNIEDLAAYNELQDAYAKQQTEAIDALNKASSENTQNIEDLAAYNELQDAYAKQQTEAID
ALNKASSENTQNIAKNQADIANNINNIYELAQQQDQHSSDIKTLAKASAANTDRIAKNKA
DADASFETLTKNQNTLIEKDKEHDKLITANKTAIDANKASADTKFAATADAITKNGNAITK
NAKSITDLGTKVDGFDSRVTALDTKHH
MC-005 (DNA)-SEQ ID NO: 66
ATGCAGGCCAAAAATGATATTACCCTGGAAGATCTGCCGTATCTGATCAAAAAAATCG
ATCAGAACGAACTGGAAGCCGATATTGGTGATATTACCGCACTGGAAAAATATCTGGC
ACTGAGCCAGTATGGAAATATTCTGGCCCTGGAAGAACTGAATAAAGCTCTGGAAGA
GCTGGATGAAGATGTGGGTTGGAATCAGAATGATATCGCCAATCTGGAAGATGATGTT
GAAACCCTGACCAAAAATCAGAATGCACTGGCAGAACAGGGTGAAGCAATTAAAGAA
GATCTGCAGGGTCTGGCAGATTTTGTTGAAGGTCAGGAAGGCAAAATTCTGCAGAACG
AAACCAGCATCAAAAAAAACACCCAGCGTAATCTGGTGAATGGCTTTGAAATTGAAA
AAAACAAAGATGCCATTGCCAAAAACAACGAAAGCATTGAAGATCTGTATGATTTTG
GTCATGAAGTTGCCGAAAGCATTGGTGAAATTCATGCACATAACGAAGCACAGAATG
AAACCCTGAAAGGTCTGATTACCAACAGCATCGAAAATACCAATAACATTACCAAAA
ACAAAGCAGATATTCAGGCGCTGGAAAATAATGTTGTGGAAGAACTGTTTAATCTGAG
CGGTCGTCTGATTGATCAGAAAGCCGATATCGATAATAACATTAACAACATTTATGAA
CTGGCACAGCAGCAGGATCAGCATAGCAGCGATATCAAAACCCTGAAAAAAAACGTT
GAAGAAGGTCTGCTGGAACTGTCTGGTCACCTGATCGATCAGAAAACTGATATTGCCC
AGAATCAGGCAAATATTCAGGATCTGGCCACCTATAATGAACTGCAGGATCAGTATGC
ACAGAAACAGACCGAAGCAATTGATGCCCTGAATAAAGCGAGCAGCGAAAACACCCA
GAATATCGAAGATCTGGCAGCATACAACGAACTGCAGGATGCCTATGCAAAACAGCA
GACTGAAGCCATCGACGCACTGAACAAGGCAAGCTCTGAAAACACGCAGAACATTGA
AGATCTGGCTGCCTATAATGAATTACAGGATGCGTATGCCAAACAGCAGACCGAAGC
GATTGATGCGCTGAACAAAGCCTCTTCTGAAAATACACAGAATATCGCCAAAAATCAG
GCCGATATTGCCAACAATATCAATAATATCTATGAACTGGCCCAGCAGCAGGATCAGC
ACTCTTCTGATATCAAAACACTGGCAAAAGCAAGCGCAGCAAATACCGATCGTATTGC
GAAAAACAAAGCCGATGCAGATGCAAGCTTTGAAACACTGACGAAAAACCAGAACAC
CCTGATTGAAAAAGATAAAGAACATGATAAACTGATCACCGCCAATAAAACCGCAAT
TGATGCAAATAAAGCCAGCGCAGATACCAAATTTGCAGCAACCGCAGATGCAATTAC
CAAAAATGGCAATGCCATCACCAAAAATGCCAAAAGCGCAAGCCATCATCATCACCA
CCACTAA
MC-005 (Protein)-(M)(UspA2 amino acids 30-519)(ASHHHHHH) SEQ ID NO: 67
MQAKNDITLEDLPYLIKKIDQNELEADIGDITALEKYLALSQYGNILALEELNKALEELDED
VGWNQNDIANLEDDVETLTKNQNALAEQGEAIKEDLQGLADFVEGQEGKILQNETSIKKN
TQRNLVNGFEIEKNKDAIAKNNESIEDLYDFGHEVAESIGEIHAHNEAQNETLKGLITNSIE
NTNNITKNKADIQALENNVVEELFNLSGRLIDQKADIDNNINNIYELAQQQDQHSSDIKTLK
KNVEEGLLELSGHLIDQKTDIAQNQANIQDLATYNELQDQYAQKQTEAIDALNKASSENT
QNIEDLAAYNELQDAYAKQQTEAIDALNKASSENTQNIEDLAAYNELQDAYAKQQTEAID
ALNKASSENTQNIAKNQADIANNINNIYELAQQQDQHSSDIKTLAKASAANTDRIAKNKA
DADASFETLTKNQNTLIEKDKEHDKLITANKTAIDANKASADTKFAATADAITKNGNAITK
NAKSASHHHHHH
MC-006 (DNA)-SEQ ID NO: 68
ATGCAGGCCAAAAATGATATTACCCTGGAAGATCTGCCGTATCTGATCAAAAAAATCG
ATCAGAACGAACTGGAAGCCGATATTGGTGATATTACCGCACTGGAAAAATATCTGGC
ACTGAGCCAGTATGGAAATATTCTGGCCCTGGAAGAACTGAATAAAGCTCTGGAAGA
GCTGGATGAAGATGTGGGTTGGAATCAGAATGATATCGCCAATCTGGAAGATGATGTT
GAAACCCTGACCAAAAATCAGAATGCACTGGCAGAACAGGGTGAAGCAATTAAAGAA
GATCTGCAGGGTCTGGCAGATTTTGTTGAAGGTCAGGAAGGCAAAATTCTGCAGAACG
AAACCAGCATCAAAAAAAACACCCAGCGTAATCTGGTGAATGGCTTTGAAATTGAAA
AAAACAAAGATGCCATTGCCAAAAACAACGAAAGCATTGAAGATCTGTATGATTTTG
GTCATGAAGTTGCCGAAAGCATTGGTGAAATTCATGCACATAACGAAGCACAGAATG
AAACCCTGAAAGGTCTGATTACCAACAGCATCGAAAATACCAATAACATTACCAAAA
ACAAAGCAGATATTCAGGCGCTGGAAAATAATGTTGTGGAAGAACTGTTTAATCTGAG
CGGTCGTCTGATTGATCAGAAAGCCGATATCGATAATAACATTAACAACATTTATGAA
CTGGCACAGCAGCAGGATCAGCATAGCAGCGATATCAAAACCCTGAAAAAAAACGTT
GAAGAAGGTCTGCTGGAACTGTCTGGTCACCTGATCGATCAGAAAACTGATATTGCCC
AGAATCAGGCAAATATTCAGGATCTGGCCACCTATAATGAACTGCAGGATCAGTATGC
ACAGAAACAGACCGAAGCAATTGATGCCCTGAATAAAGCGAGCAGCGAAAACACCCA
GAATATCGAAGATCTGGCAGCATACAACGAACTGCAGGATGCCTATGCAAAACAGCA
GACTGAAGCCATCGACGCACTGAACAAGGCAAGCTCTGAAAACACGCAGAACATTGA
AGATCTGGCTGCCTATAATGAATTACAGGATGCGTATGCCAAACAGCAGACCGAAGC
GATTGATGCGCTGAACAAAGCCTCTTCTGAAAATACACAGAATATCGCCAAAAATCAG
GCCGATATTGCCAACAATATCAATAATATCTATGAACTGGCCCAGCAGCAGGATCAGC
ACTCTTCTGATATCAAAACACTGGCAAAAGCAAGCGCAGCAAATACCGATCGTATTGC
GAAAAACAAAGCCGATGCAGATGCAAGCTTTGAAACACTGACGAAAAACCAGAACAC
CCTGATTGAAAAAGATAAAGAACATGATAAACTGATCACCGCCAATAAAACCGCAAT
TGATGCAAATAAAGCCAGCGCAGATACCAAATTTGCAGCAACCGCAGATGCAATTAC
CAAAAATGGCAATGCCATCACCAAAAATGCCAAAAGCTAA
MC-006 (Protein)-(M)(UspA2 amino acids 30-519) SEQ ID NO: 69
MQAKNDITLEDLPYLIKKIDQNELEADIGDITALEKYLALSQYGNILALEELNKALEELDED
VGWNQNDIANLEDDVETLTKNQNALAEQGEAIKEDLQGLADFVEGQEGKILQNETSIKKN
TQRNLVNGFEIEKNKDAIAKNNESIEDLYDFGHEVAESIGEIHAHNEAQNETLKGLITNSIE
NTNNITKNKADIQALENNVVEELFNLSGRLIDQKADIDNNINNIYELAQQQDQHSSDIKTLK
KNVEEGLLELSGHLIDQKTDIAQNQANIQDLATYNELQDQYAQKQTEAIDALNKASSENT
QNIEDLAAYNELQDAYAKQQTEAIDALNKASSENTQNIEDLAAYNELQDAYAKQQTEAID
ALNKASSENTQNIAKNQADIANNINNIYELAQQQDQHSSDIKTLAKASAANTDRIAKNKA
DADASFETLTKNQNTLIEKDKEHDKLITANKTAIDANKASADTKFAATADAITKNGNAITK
NAKS
MC-007 (DNA)-SEQ ID NO: 70
ATGCAGGCCAAAAATGATATTACCCTGGAAGATCTGCCGTATCTGATCAAAAAAATCG
ATCAGAACGAACTGGAAGCCGATATTGGTGATATTACCGCACTGGAAAAATATCTGGC
ACTGAGCCAGTATGGAAATATTCTGGCCCTGGAAGAACTGAATAAAGCTCTGGAAGA
GCTGGATGAAGATGTGGGTTGGAATCAGAATGATATCGCCAATCTGGAAGATGATGTT
GAAACCCTGACCAAAAATCAGAATGCACTGGCAGAACAGGGTGAAGCAATTAAAGAA
GATCTGCAGGGTCTGGCAGATTTTGTTGAAGGTCAGGAAGGCAAAATTCTGCAGAACG
AAACCAGCATCAAAAAAAACACCCAGCGTAATCTGGTGAATGGCTTTGAAATTGAAA
AAAACAAAGATGCCATTGCCAAAAACAACGAAAGCATTGAAGATCTGTATGATTTTG
GTCATGAAGTTGCCGAAAGCATTGGTGAAATTCATGCACATAACGAAGCACAGAATG
AAACCCTGAAAGGTCTGATTACCAACAGCATCGAAAATACCAATAACATTACCAAAA
ACAAAGCAGATATTCAGGCGCTGGAAAATAATGTTGTGGAAGAACTGTTTAATCTGAG
CGGTCGTCTGATTGATCAGAAAGCCGATATCGATAATAACATTAACAACATTTATGAA
CTGGCACAGCAGCAGGATCAGCATAGCAGCGATATCAAAACCCTGAAAAAAAACGTT
GAAGAAGGTCTGCTGGAACTGTCTGGTCACCTGATCGATCAGAAAACTGATATTGCCC
AGAATCAGGCAAATATTCAGGATCTGGCCACCTATAATGAACTGCAGGATCAGTATGC
ACAGAAACAGACCGAAGCAATTGATGCCCTGAATAAAGCGAGCAGCGAAAACACCCA
GAATATCGAAGATCTGGCAGCATACAACGAACTGCAGGATGCCTATGCAAAACAGCA
GACTGAAGCCATCGACGCACTGAACAAGGCAAGCTCTGAAAACACGCAGAACATTGA
AGATCTGGCTGCCTATAATGAATTACAGGATGCGTATGCCAAACAGCAGACCGAAGC
GATTGATGCGCTGAACAAAGCCTCTTCTGAAAATACACAGAATATCGCCAAAAATCAG
GCCGATATTGCCAACAATATCAATAATATCTATGAACTGGCCCAGCAGCAGGATCAGC
ACTCTTCTGATATCAAAACACTGGCAAAAGCAAGCGCAGCAAATACCGATCGTATTGC
GAAAAACAAAGCCGATGCAGATGCAAGCTTTGAAACACTGACGAAAAACCAGAACAC
CCTGATTGAAAAAGATAAAGAACATGATAAACTGATCACCGCCAATAAAACCGCAAT
TGATGCAAATAAAGCCAGCGCAGATACCAAATTTGCAGCAACCGCAGATGCAATTAC
CAAAAATGGCAATGCCATCACCAAAAATGCCAAAAGCATTACCGATCTGGGCACCAA
AGTTGATGGTTTTGATAGCCGTGTGACCGCACTGGATACCAAAGTTAATGCATTTGAT
GGTCGTATTACCGCTCTGGATAGTAAAGTTGAAAATGGTATGGCAGCACAGGCAGCA
GCAAGCCATCATCATCACCACCACTAA
MC-007 (Protein)-(M)(UspA2 amino acids 30-564)(ASHHHHHH) SEQ ID NO: 71
MQAKNDITLEDLPYLIKKIDQNELEADIGDITALEKYLALSQYGNILALEELNKALEELDED
VGWNQNDIANLEDDVETLTKNQNALAEQGEAIKEDLQGLADFVEGQEGKILQNETSIKKN
TQRNLVNGFEIEKNKDAIAKNNESIEDLYDFGHEVAESIGEIHAHNEAQNETLKGLITNSIE
NTNNITKNKADIQALENNVVEELFNLSGRLIDQKADIDNNINNIYELAQQQDQHSSDIKTLK
KNVEEGLLELSGHLIDQKTDIAQNQANIQDLATYNELQDQYAQKQTEAIDALNKASSENT
QNIEDLAAYNELQDAYAKQQTEAIDALNKASSENTQNIEDLAAYNELQDAYAKQQTEAID
ALNKASSENTQNIAKNQADIANNINNIYELAQQQDQHSSDIKTLAKASAANTDRIAKNKA
DADASFETLTKNQNTLIEKDKEHDKLITANKTAIDANKASADTKFAATADAITKNGNAITK
NAKSITDLGTKVDGFDSRVTALDTKVNAFDGRITALDSKVENGMAAQAAASHHHHHH
MC-008 (DNA)-SEQ ID NO: 72
ATGCAGGCCAAAAATGATATTACCCTGGAAGATCTGCCGTATCTGATCAAAAAAATCG
ATCAGAACGAACTGGAAGCCGATATTGGTGATATTACCGCACTGGAAAAATATCTGGC
ACTGAGCCAGTATGGAAATATTCTGGCCCTGGAAGAACTGAATAAAGCTCTGGAAGA
GCTGGATGAAGATGTGGGTTGGAATCAGAATGATATCGCCAATCTGGAAGATGATGTT
GAAACCCTGACCAAAAATCAGAATGCACTGGCAGAACAGGGTGAAGCAATTAAAGAA
GATCTGCAGGGTCTGGCAGATTTTGTTGAAGGTCAGGAAGGCAAAATTCTGCAGAACG
AAACCAGCATCAAAAAAAACACCCAGCGTAATCTGGTGAATGGCTTTGAAATTGAAA
AAAACAAAGATGCCATTGCCAAAAACAACGAAAGCATTGAAGATCTGTATGATTTTG
GTCATGAAGTTGCCGAAAGCATTGGTGAAATTCATGCACATAACGAAGCACAGAATG
AAACCCTGAAAGGTCTGATTACCAACAGCATCGAAAATACCAATAACATTACCAAAA
ACAAAGCAGATATTCAGGCGCTGGAAAATAATGTTGTGGAAGAACTGTTTAATCTGAG
CGGTCGTCTGATTGATCAGAAAGCCGATATCGATAATAACATTAACAACATTTATGAA
CTGGCACAGCAGCAGGATCAGCATAGCAGCGATATCAAAACCCTGAAAAAAAACGTT
GAAGAAGGTCTGCTGGAACTGTCTGGTCACCTGATCGATCAGAAAACTGATATTGCCC
AGAATCAGGCAAATATTCAGGATCTGGCCACCTATAATGAACTGCAGGATCAGTATGC
ACAGAAACAGACCGAAGCAATTGATGCCCTGAATAAAGCGAGCAGCGAAAACACCCA
GAATATCGAAGATCTGGCAGCATACAACGAACTGCAGGATGCCTATGCAAAACAGCA
GACTGAAGCCATCGACGCACTGAACAAGGCAAGCTCTGAAAACACGCAGAACATTGA
AGATCTGGCTGCCTATAATGAATTACAGGATGCGTATGCCAAACAGCAGACCGAAGC
GATTGATGCGCTGAACAAAGCCTCTTCTGAAAATACACAGAATATCGCCAAAAATCAG
GCCGATATTGCCAACAATATCAATAATATCTATGAACTGGCCCAGCAGCAGGATCAGC
ACTCTTCTGATATCAAAACACTGGCAAAAGCAAGCGCAGCAAATACCGATCGTATTGC
GAAAAACAAAGCCGATGCAGATGCAAGCTTTGAAACACTGACGAAAAACCAGAACAC
CCTGATTGAAAAAGATAAAGAACATGATAAACTGATCACCGCCAATAAAACCGCAAT
TGATGCAAATAAAGCCAGCGCAGATACCAAATTTGCAGCAACCGCAGATGCAATTAC
CAAAAATGGCAATGCCATCACCAAAAATGCCAAAAGCATTACCGATCTGGGCACCAA
AGTTGATGGTTTTGATAGCCGTGTGACCGCACTGGATACCAAAGTTAATGCATTTGAT
GGTCGTATTACCGCTCTGGATAGTAAAGTTGAAAATGGTATGGCAGCACAGGCAGCAC
ACCACTAA
MC-008 (Protein)-(M)(UspA2 30-564)(HH) SEQ ID NO: 73
MQAKNDITLEDLPYLIKKIDQNELEADIGDITALEKYLALSQYGNILALEELNKALEELDED
VGWNQNDIANLEDDVETLTKNQNALAEQGEAIKEDLQGLADFVEGQEGKILQNETSIKKN
TQRNLVNGFEIEKNKDAIAKNNESIEDLYDFGHEVAESIGEIHAHNEAQNETLKGLITNSIE
NTNNITKNKADIQALENNVVEELFNLSGRLIDQKADIDNNINNIYELAQQQDQHSSDIKTLK
KNVEEGLLELSGHLIDQKTDIAQNQANIQDLATYNELQDQYAQKQTEAIDALNKASSENT
QNIEDLAAYNELQDAYAKQQTEAIDALNKASSENTQNIEDLAAYNELQDAYAKQQTEAID
ALNKASSENTQNIAKNQADIANNINNIYELAQQQDQHSSDIKTLAKASAANTDRIAKNKA
DADASFETLTKNQNTLIEKDKEHDKLITANKTAIDANKASADTKFAATADAITKNGNAITK
NAKSITDLGTKVDGFDSRVTALDTKVNAFDGRITALDSKVENGMAAQAAHH
MC-009 (DNA)-SEQ ID NO: 74
ATGGCGAAAAATGATATTACCCTGGAAGATCTGCCGTATCTGATCAAAAAAATCGATC
AGAACGAACTGGAAGCCGATATTGGTGATATTACCGCACTGGAAAAATATCTGGCACT
GAGCCAGTATGGAAATATTCTGGCCCTGGAAGAACTGAATAAAGCTCTGGAAGAGCT
GGATGAAGATGTGGGTTGGAATCAGAATGATATCGCCAATCTGGAAGATGATGTTGA
AACCCTGACCAAAAATCAGAATGCACTGGCAGAACAGGGTGAAGCAATTAAAGAAGA
TCTGCAGGGTCTGGCAGATTTTGTTGAAGGTCAGGAAGGCAAAATTCTGCAGAACGAA
ACCAGCATCAAAAAAAACACCCAGCGTAATCTGGTGAATGGCTTTGAAATTGAAAAA
AACAAAGATGCCATTGCCAAAAACAACGAAAGCATTGAAGATCTGTATGATTTTGGTC
ATGAAGTTGCCGAAAGCATTGGTGAAATTCATGCACATAACGAAGCACAGAATGAAA
CCCTGAAAGGTCTGATTACCAACAGCATCGAAAATACCAATAACATTACCAAAAACA
AAGCAGATATTCAGGCGCTGGAAAATAATGTTGTGGAAGAACTGTTTAATCTGAGCGG
TCGTCTGATTGATCAGAAAGCCGATATCGATAATAACATTAACAACATTTATGAACTG
GCACAGCAGCAGGATCAGCATAGCAGCGATATCAAAACCCTGAAAAAAAACGTTGAA
GAAGGTCTGCTGGAACTGTCTGGTCACCTGATCGATCAGAAAACTGATATTGCCCAGA
ATCAGGCAAATATTCAGGATCTGGCCACCTATAATGAACTGCAGGATCAGTATGCACA
GAAACAGACCGAAGCAATTGATGCCCTGAATAAAGCGAGCAGCGAAAACACCCAGAA
TATCGAAGATCTGGCAGCATACAACGAACTGCAGGATGCCTATGCAAAACAGCAGAC
TGAAGCCATCGACGCACTGAACAAGGCAAGCTCTGAAAACACGCAGAACATTGAAGA
TCTGGCTGCCTATAATGAATTACAGGATGCGTATGCCAAACAGCAGACCGAAGCGATT
GATGCGCTGAACAAAGCCTCTTCTGAAAATACACAGAATATCGCCAAAAATCAGGCC
GATATTGCCAACAATATCAATAATATCTATGAACTGGCCCAGCAGCAGGATCAGCACT
CTTCTGATATCAAAACACTGGCAAAAGCAAGCGCAGCAAATACCGATCGTATTGCGA
AAAACAAAGCCGATGCAGATGCAAGCTTTGAAACACTGACGAAAAACCAGAACACCC
TGATTGAAAAAGATAAAGAACATGATAAACTGATCACCGCCAATAAAACCGCAATTG
ATGCAAATAAAGCCAGCGCAGATACCAAATTTGCAGCAACCGCAGATGCAATTACCA
AAAATGGCAATGCCATCACCAAAAATGCCAAAAGCATTACCGATCTGGGCACCAAAG
TTGATGGTTTTGATAGCCGTGTGACCGCACTGGATACCAAAGTTAATGCATTTGATGGT
CGTATTACCGCTCTGGATAGTAAAGTTGAAAATGGTATGGCAGCACAGGCAGCACACC
ACTAA
MC-009 (Protein)-(M)(UspA2 31-564)(HH) SEQ ID NO: 75
MAKNDITLEDLPYLIKKIDQNELEADIGDITALEKYLALSQYGNILALEELNKALEELDEDV
GWNQNDIANLEDDVETLTKNQNALAEQGEAIKEDLQGLADFVEGQEGKILQNETSIKKNT
QRNLVNGFEIEKNKDAIAKNNESIEDLYDFGHEVAESIGEIHAHNEAQNETLKGLITNSIEN
TNNITKNKADIQALENNVVEELFNLSGRLIDQKADIDNNINNIYELAQQQDQHSSDIKTLKK
NVEEGLLELSGHLIDQKTDIAQNQANIQDLATYNELQDQYAQKQTEAIDALNKASSENTQ
NIEDLAAYNELQDAYAKQQTEAIDALNKASSENTQNIEDLAAYNELQDAYAKQQTEAIDA
LNKASSENTQNIAKNQADIANNINNIYELAQQQDQHSSDIKTLAKASAANTDRIAKNKAD
ADASFETLTKNQNTLIEKDKEHDKLITANKTAIDANKASADTKFAATADAITKNGNAITKN
AKSITDLGTKVDGFDSRVTALDTKVNAFDGRITALDSKVENGMAAQAAHH
MC-010 (DNA)-SEQ ID NO: 76
ATGCAGGCCAAAAATGATATTACCCTGGAAGATCTGCCGTATCTGATCAAAAAAATCG
ATCAGAACGAACTGGAAGCCGATATTGGTGATATTACCGCACTGGAAAAATATCTGGC
ACTGAGCCAGTATGGAAATATTCTGGCCCTGGAAGAACTGAATAAAGCTCTGGAAGA
GCTGGATGAAGATGTGGGTTGGAATCAGAATGATATCGCCAATCTGGAAGATGATGTT
GAAACCCTGACCAAAAATCAGAATGCACTGGCAGAACAGGGTGAAGCAATTAAAGAA
GATCTGCAGGGTCTGGCAGATTTTGTTGAAGGTCAGGAAGGCAAAATTCTGCAGAACG
AAACCAGCATCAAAAAAAACACCCAGCGTAATCTGGTGAATGGCTTTGAAATTGAAA
AAAACAAAGATGCCATTGCCAAAAACAACGAAAGCATTGAAGATCTGTATGATTTTG
GTCATGAAGTTGCCGAAAGCATTGGTGAAATTCATGCACATAACGAAGCACAGAATG
AAACCCTGAAAGGTCTGATTACCAACAGCATCGAAAATACCAATAACATTACCAAAA
ACAAAGCAGATATTCAGGCGCTGGAAAATAATGTTGTGGAAGAACTGTTTAATCTGAG
CGGTCGTCTGATTGATCAGAAAGCCGATATCGATAATAACATTAACAACATTTATGAA
CTGGCACAGCAGCAGGATCAGCATAGCAGCGATATCAAAACCCTGAAAAAAAACGTT
GAAGAAGGTCTGCTGGAACTGTCTGGTCACCTGATCGATCAGAAAACTGATATTGCCC
AGAATCAGGCAAATATTCAGGATCTGGCCACCTATAATGAACTGCAGGATCAGTATGC
ACAGAAACAGACCGAAGCAATTGATGCCCTGAATAAAGCGAGCAGCGAAAACACCCA
GAATATCGAAGATCTGGCAGCATACAACGAACTGCAGGATGCCTATGCAAAACAGCA
GACTGAAGCCATCGACGCACTGAACAAGGCAAGCTCTGAAAACACGCAGAACATTGA
AGATCTGGCTGCCTATAATGAATTACAGGATGCGTATGCCAAACAGCAGACCGAAGC
GATTGATGCGCTGAACAAAGCCTCTTCTGAAAATACACAGAATATCGCCAAAAATCAG
GCCGATATTGCCAACAATATCAATAATATCTATGAACTGGCCCAGCAGCAGGATCAGC
ACTCTTCTGATATCAAAACACTGGCAAAAGCAAGCGCAGCAAATACCGATCGTATTGC
GAAAAACAAAGCCGATGCAGATGCAAGCTTTGAAACACTGACGAAAAACCAGAACAC
CCTGATTGAAAAAGATAAAGAACATGATAAACTGATCACCGCCAATAAAACCGCAAT
TGATGCAAATAAAGCCAGCGCAGATACCAAATTTGCAGCAACCGCAGATGCAATTAC
CAAAAATGGCAATGCCATCACCAAAAATGCCAAAAGCATTACCGATCTGGGCACCAA
AGTTGATGGTTTTGATAGCCGTGTGACCGCACTGGATACCAAAGTTAATGCATTTGAT
GGTCGTATTACCGCTCTGGATAGTAAAGTTGAAAATGGTATGGCAGCACAGGCAGCAT
AA
MC-010 (Protein)-(M)(UspA2 amino acids 30-564) SEQ ID NO: 77
MQAKNDITLEDLPYLIKKIDQNELEADIGDITALEKYLALSQYGNILALEELNKALEELDED
VGWNQNDIANLEDDVETLTKNQNALAEQGEAIKEDLQGLADFVEGQEGKILQNETSIKKN
TQRNLVNGFEIEKNKDAIAKNNESIEDLYDFGHEVAESIGEIHAHNEAQNETLKGLITNSIE
NTNNITKNKADIQALENNVVEELFNLSGRLIDQKADIDNNINNIYELAQQQDQHSSDIKTLK
KNVEEGLLELSGHLIDQKTDIAQNQANIQDLATYNELQDQYAQKQTEAIDALNKASSENT
QNIEDLAAYNELQDAYAKQQTEAIDALNKASSENTQNIEDLAAYNELQDAYAKQQTEAID
ALNKASSENTQNIAKNQADIANNINNIYELAQQQDQHSSDIKTLAKASAANTDRIAKNKA
DADASFETLTKNQNTLIEKDKEHDKLITANKTAIDANKASADTKFAATADAITKNGNAITK
NAKSITDLGTKVDGFDSRVTALDTKVNAFDGRITALDSKVENGMAAQAA
MC-011 (DNA)-SEQ ID NO: 78
ATGGCGAAAAATGATATTACCCTGGAAGATCTGCCGTATCTGATCAAAAAAATCGATC
AGAACGAACTGGAAGCCGATATTGGTGATATTACCGCACTGGAAAAATATCTGGCACT
GAGCCAGTATGGAAATATTCTGGCCCTGGAAGAACTGAATAAAGCTCTGGAAGAGCT
GGATGAAGATGTGGGTTGGAATCAGAATGATATCGCCAATCTGGAAGATGATGTTGA
AACCCTGACCAAAAATCAGAATGCACTGGCAGAACAGGGTGAAGCAATTAAAGAAGA
TCTGCAGGGTCTGGCAGATTTTGTTGAAGGTCAGGAAGGCAAAATTCTGCAGAACGAA
ACCAGCATCAAAAAAAACACCCAGCGTAATCTGGTGAATGGCTTTGAAATTGAAAAA
AACAAAGATGCCATTGCCAAAAACAACGAAAGCATTGAAGATCTGTATGATTTTGGTC
ATGAAGTTGCCGAAAGCATTGGTGAAATTCATGCACATAACGAAGCACAGAATGAAA
CCCTGAAAGGTCTGATTACCAACAGCATCGAAAATACCAATAACATTACCAAAAACA
AAGCAGATATTCAGGCGCTGGAAAATAATGTTGTGGAAGAACTGTTTAATCTGAGCGG
TCGTCTGATTGATCAGAAAGCCGATATCGATAATAACATTAACAACATTTATGAACTG
GCACAGCAGCAGGATCAGCATAGCAGCGATATCAAAACCCTGAAAAAAAACGTTGAA
GAAGGTCTGCTGGAACTGTCTGGTCACCTGATCGATCAGAAAACTGATATTGCCCAGA
ATCAGGCAAATATTCAGGATCTGGCCACCTATAATGAACTGCAGGATCAGTATGCACA
GAAACAGACCGAAGCAATTGATGCCCTGAATAAAGCGAGCAGCGAAAACACCCAGAA
TATCGAAGATCTGGCAGCATACAACGAACTGCAGGATGCCTATGCAAAACAGCAGAC
TGAAGCCATCGACGCACTGAACAAGGCAAGCTCTGAAAACACGCAGAACATTGAAGA
TCTGGCTGCCTATAATGAATTACAGGATGCGTATGCCAAACAGCAGACCGAAGCGATT
GATGCGCTGAACAAAGCCTCTTCTGAAAATACACAGAATATCGCCAAAAATCAGGCC
GATATTGCCAACAATATCAATAATATCTATGAACTGGCCCAGCAGCAGGATCAGCACT
CTTCTGATATCAAAACACTGGCAAAAGCAAGCGCAGCAAATACCGATCGTATTGCGA
AAAACAAAGCCGATGCAGATGCAAGCTTTGAAACACTGACGAAAAACCAGAACACCC
TGATTGAAAAAGATAAAGAACATGATAAACTGATCACCGCCAATAAAACCGCAATTG
ATGCAAATAAAGCCAGCGCAGATACCAAATTTGCAGCAACCGCAGATGCAATTACCA
AAAATGGCAATGCCATCACCAAAAATGCCAAAAGCATTACCGATCTGGGCACCAAAG
TTGATGGTTTTGATAGCCGTGTGACCGCACTGGATACCAAAGCAAGCCATCATCATCA
CCACCACTAA
MC-011 (Protein)-(M)(UspA2 amino acids 31-540)(ASHHHHHH) SEQ ID NO: 79
MAKNDITLEDLPYLIKKIDQNELEADIGDITALEKYLALSQYGNILALEELNKALEELDEDV
GWNQNDIANLEDDVETLTKNQNALAEQGEAIKEDLQGLADFVEGQEGKILQNETSIKKNT
QRNLVNGFEIEKNKDAIAKNNESIEDLYDFGHEVAESIGEIHAHNEAQNETLKGLITNSIEN
TNNITKNKADIQALENNVVEELFNLSGRLIDQKADIDNNINNIYELAQQQDQHSSDIKTLKK
NVEEGLLELSGHLIDQKTDIAQNQANIQDLATYNELQDQYAQKQTEAIDALNKASSENTQ
NIEDLAAYNELQDAYAKQQTEAIDALNKASSENTQNIEDLAAYNELQDAYAKQQTEAIDA
LNKASSENTQNIAKNQADIANNINNIYELAQQQDQHSSDIKTLAKASAANTDRIAKNKAD
ADASFETLTKNQNTLIEKDKEHDKLITANKTAIDANKASADTKFAATADAITKNGNAITKN
AKSITDLGTKVDGFDSRVTALDTKASHHHHHH
DNA Sequence for UspA2 from strain ATCC 25238 (SEQ ID NO: 1)-SEQ ID NO: 80
ATGAAAACCATGAAACTTCTCCCTCTAAAAATCGCTGTAACCAGTGCCATGATTATTG
GCTTGGGTGCGGCATCTACTGCGAATGCGCAGGCTAAAAATGATATAACTTTAGAGG
ATTTACCATATTTAATAAAAAAGATTGACCAAAATGAATTGGAAGCAGATATCGGA
GATATTACTGCTCTTGAAAAGTATCTAGCACTTAGCCAGTATGGCAATATTTTAGCTC
TAGAAGAGCTCAACAAGGCTCTAGAAGAGCTCGACGAGGATGTTGGATGGAATCAG
AATGATATTGCAAACTTGGAAGATGATGTTGAAACGCTCACCAAAAATCAAAATGC
TTTGGCTGAACAAGGTGAGGCAATTAAAGAAGATCTTCAAGGGCTTGCAGATTTTGT
AGAAGGGCAAGAGGGTAAAATTCTACAAAATGAAACTTCAATTAAAAAAAATACTC
AGAGAAACCTTGTCAATGGGTTTGAGATTGAGAAAAATAAAGATGCTATTGCTAAA
AACAATGAGTCTATCGAAGATCTTTATGATTTTGGTCATGAGGTTGCAGAAAGTATA
GGCGAGATACATGCTCATAATGAAGCGCAAAATGAAACTCTTAAAGGCTTGATAAC
AAACAGTATTGAGAATACTAATAATATTACCAAAAACAAAGCTGACATCCAAGCAC
TTGAAAACAATGTCGTAGAAGAACTATTCAATCTAAGCGGTCGCCTAATTGATCAAA
AAGCAGATATTGATAATAACATCAACAATATCTATGAGCTGGCACAACAGCAAGAT
CAGCATAGCTCTGATATCAAAACACTTAAAAAAAATGTCGAAGAAGGTTTGTTGGA
GCTAAGCGGTCACCTAATTGATCAAAAAACAGATATTGCTCAAAACCAAGCTAACA
TCCAAGATCTGGCCACTTACAACGAGCTACAAGACCAGTATGCTCAAAAGCAAACC
GAAGCGATTGACGCTCTAAATAAAGCAAGCTCTGAGAATACACAAAACATCGAAGA
TCTGGCCGCTTACAACGAGCTACAAGATGCCTATGCCAAACAGCAAACCGAAGCAA
TTGACGCTCTAAATAAAGCAAGCTCTGAGAATACACAAAACATCGAAGATCTGGCC
GCTTACAACGAGCTACAAGATGCCTATGCCAAACAGCAAACCGAAGCCATTGACGC
TCTAAATAAAGCAAGCTCTGAGAATACACAAAACATTGCTAAAAACCAAGCGGATA
TTGCTAATAACATCAACAATATCTATGAGCTGGCACAACAGCAAGATCAGCATAGCT
CTGATATCAAAACCTTGGCAAAAGCAAGTGCTGCCAATACTGATCGTATTGCTAAAA
ACAAAGCCGATGCTGATGCAAGTTTTGAAACGCTCACCAAAAATCAAAATACTTTGA
TTGAAAAAGATAAAGAGCATGACAAATTAATTACTGCAAACAAAACTGCGATTGAT
GCCAATAAAGCATCTGCGGATACCAAGTTTGCAGCGACAGCAGACGCCATTACCAA
AAATGGAAATGCTATCACTAAAAACGCAAAATCTATCACTGATTTGGGCACTAAAGT
GGATGGTTTTGACAGTCGTGTAACTGCATTAGACACCAAAGTCAATGCCTTTGATGG
TCGTATCACAGCTTTAGACAGTAAAGTTGAAAACGGTATGGCTGCCCAAGCTGCCCT
AAGTGGTCTATTCCAGCCTTATAGCGTTGGTAAGTTTAATGCGACCGCTGCACTTGG
TGGCTATGGCTCAAAATCTGCGGTTGCTATCGGTGCTGGCTATCGTGTGAATCCAAA
TCTGGCGTTTAAAGCTGGTGCGGCGATTAATACCAGTGGTAATAAAAAAGGCTCTTA
TAACATCGGTGTGAATTACGAGTTCTAA
SEQ ID NO: 81-Full Epitope of UspA2 pAb
AATADAITKNGNAITKNAKSITDLGTKVDGFDSRVTALDTKVNAFDGRITAL
SEQ ID NO: 82
YNELQD-[A/Q]-YA-[QK/KQ]-QTE
SEQ ID NO: 83
YNELQDAYAQKQTE
SEQ ID NO: 84
YNELQDAYAKQQTE
SEQ ID NO: 85
YNELQDQYAQKQTE
SEQ ID NO: 86
YNELQDQYAKQQTE
SEQ ID NO: 87 (CDRH1)
SYWMH
SEQ ID NO: 88 (CDRH2)
AIYPGNSDTSYNQKFKG
SEQ ID NO: 89 (CDRH3)
LRFLDAY
SEQ ID NO: 90 (CDRL1)
RASSSVSYMH
SEQ ID NO: 91 (CDRL2)
ATSNLAS
SEQ ID NO: 92 (CDRL3)
QQWSSNPPT
SEQ ID NO: 93 (Variable Heavy [VH] Chain-FHUSPA2/10)
GAGGTTCAGCTCCAGCAGTCTGGGACTGTGCTGGCAAGGCCTGGGGCTTCCGTGAAGA
TGTCCTGCAAGGCTTCTGGCTACAGCTTTACCAGCTACTGGATGCACTGGGTAAAACA
GAGGCCTGGACAGGGTCTAGAATGGATTGGTGCTATTTATCCTGGAAATAGTGATACT
AGCTACAACCAGAAGTTCAAGGGCAAGGCCAAACTGACTGCAGTCACATCCGCCAGC
ACTGCCTACATGGAGCTCAGCAACCTGACGAATGAGGACTCTGCGGTCTATTCCTGTA
CATTACTACGTTTCCTCGATGCTTACTGGGGCCAAGGGACTCTGGTCACTGTCTCTGCA
G
SEQ ID NO: 94 (VH Chain of FHUSPA2/10-AA Seq)
EVQLQQSGTVLARPGASVKMSCKASGYSFTSYWMHWVKQRPGQGLEWIGAIYPGNSDTS
YNQKFKGKAKLTAVTSASTAYMELSNLTNEDSAVYSCTLLRFLDAYWGQGTLVTVSA
SEQ ID NO: 95 (Variable Light [VL] Chain-FHUSPA2/10)
CAAATTGTTCTCTCCCAGTCTCCAGCAATCCTGTCTGCATCTCCAGGGGAGAAGGTCA
CAATGACTTGCAGGGCCAGCTCAAGTGTAAGTTACATGCACTGGTACCAGCAGAAGCC
AGGATCCTCCCCCAAACCCTGGATTTATGCCACATCCAACCTGGCTTCTGGAGTCCCTG
CTCGCTTCAGTGGCAGTGGGTCTGGGACCTCTTACTCTCTCACAATCAGCAGAGTGGA
GGCTGAAGATGCTGCCACTTATTACTGCCAGCAGTGGAGTAGTAACCCACCCACGTTC
GGAGGGGGGTCCAAGCTGGAAATAAAA
SEQ ID NO: 96 (VL Chain of FHUSPA2/10-AA Seq)
QIVLSQSPAILSASPGEKVTMTCRASSSVSYMHWYQQKPGSSPKPWIYATSNLASGVPARF
SGSGSGTSYSLTISRVEAEDAATYYCQQWSSNPPTFGGGSKLEIK

1. (canceled)

2. (canceled)

3. (canceled)

4. An assay comprising the steps of;
(a) contacting a test sample comprising UspA2 with a first antibody and a second antibody to form a first antibody-UspA2-second antibody complex and
(b) detecting or measuring the amount of said first antibody-UspA2-second antibody complex, wherein said test sample comprising UspA2 is an UspA2 antigen which has been manufactured for administration in a vaccine.

5. The assay according to claim 4, wherein at least one of said first antibody or said second antibody is immobilized on a solid support.

6. The assay according to claim 5 wherein said first antibody and said second antibody bind non-overlapping epitopes on UspA2.

7. The assay according to claim 4 wherein at least one of said first antibody or said second antibody is a monoclonal antibody and comprises a VH region comprising a sequence at least 80% identical to SEQ ID NO: 94 and a VL region comprising a sequence at least 80% identical to SEQ ID NO: 96.

8. The assay according to claim 4 wherein at least one of said first antibody or said second antibody is a polyclonal antibody, which is capable of binding to UspA2.

9. The assay according to claim 4 wherein a) said first antibody is the monoclonal antibody as described in claim 7 and is immobilized on a solid support and said second antibody is a polyclonal antibody and is not immobilized on a solid support; or b) said first antibody is a polyclonal antibody and is immobilized on a solid support and said second antibody is the monoclonal antibody as described in claim 7 and is not immobilized on a solid support.

10. The assay according to claim 9 wherein said first antibody comprises a VH region of SEQ ID NO: 94 and a VL region of SEQ ID NO: 96 and is immobilized on a solid support and said second antibody is a polyclonal antibody which binds to UspA2 and is not immobilized on a solid support.

11. The assay according to claim 9 wherein said second antibody that is not immobilized on said solid support is unlabeled.

12. The assay according to claim 11, wherein said unlabeled second antibody is contacted with an enzyme-labelled third antibody that binds to said unlabeled second antibody to detect the formation of the first antibody-UspA2-second antibody complex.

13. The assay according to claim 4 further comprising comparing the amount of the first antibody-UspA2-second antibody complexes formed with a test sample comprising UspA2 with the amount of the first antibody-UspA2-second antibody complexes formed with a reference sample.

14. The assay of claim 4 wherein the test sample comprising UspA2 comprises an UspA2 antigen with a sequence with at least 80% identity to any one of SEQ ID NO: 59 (MC-001), SEQ ID NO: 61 (MC-002), SEQ ID NO: 63 (MC-003), SEQ ID NO: 65 (MC-004), SEQ ID NO: 67 (MC-005), SEQ ID NO: 69 (MC-006), SEQ ID NO: 71 (MC-007), SEQ ID NO: 73 (MC-008), SEQ ID NO: 75 (MC-009), SEQ ID NO: 77 (MC-010) or SEQ ID NO: 79 (MC-011).

15. (canceled)

16. The assay according to claim 4 wherein the assay is a sandwich ELISA assay.

17. The assay according to claim 8 wherein at least one of said first antibody or said second antibody is an avian, rabbit or goat polyclonal antibody.

18. The assay according to claim 10 wherein the second antibody is a rabbit polyclonal antibody.

19. The assay according to claim 12 wherein said enzyme-labelled third antibody is peroxidase labelled.

20. A method for in vitro analysis of a test antigen, comprising the steps of:
(i) performing the assay of claim 4 on a test antigen and a reference sample of known potency; and
(ii) comparing the results from step (i) to determine the potency of the test antigen relative to the reference sample.

21. A method for detecting or measuring a change in the conformation of UspA2 comprising:
(a) contacting a test sample comprising UspA2 with a first antibody and a second antibody to form a first antibody-UspA2-second antibody complex; and
(b) detecting or measuring a change in the conformation of UspA2 by detecting or measuring the amount of said first antibody-UspA2-second antibody complex.

22. The method of claim 21 wherein the antibody which binds to UspA2 comprises a VH region comprising a sequence at least 80% identical to SEQ ID NO: 94 and a VL region comprising a sequence at least 80% identical to SEQ ID NO: 96.

23. The method of claim 21 wherein the UspA2 which is present in a test sample comprises a sequence with at least 80% identity to any one of SEQ ID NO: 59 (MC-001), SEQ ID NO: 61 (MC-002), SEQ ID NO: 63 (MC-003), SEQ ID NO: 65 (MC-004), SEQ ID NO: 67 (MC-005), SEQ ID NO: 69 (MC-006), SEQ ID NO: 71 (MC-007), SEQ ID NO: 73 (MC-008), SEQ ID NO: 75 (MC-009), SEQ ID NO: 77 (MC-010) or SEQ ID NO: 79 (MC-011).

24. The method of claim 21 wherein the antibody which binds to UspA2 binds an epitope within the consensus sequence of SEQ ID NO: 82 (e.g. SEQ ID NO: 83, 84, 85, 86) and comprises a VH region comprising a sequence at least 80% identical to SEQ ID NO: 94 and a VL region comprising a sequence at least 80% identical to SEQ ID NO: 96.


SEQUENCE LISTING (125 sequences)

SEQ ID NO: 1 - protein, 630 aa, Moraxella catarrhalis
MKTMKLLPLKIAVTSAMIIGLGAASTANAQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 2 - protein, 613 aa, Moraxella catarrhalis
MKTMKLLPLKIAVTSAMIIGLGAASTANAQSRDRSLEDIQDSISKLVQDDINTLKQDQQKMNKYLLLNQLANTLITDELNNNVIKNTNSIEALGDEIGWLENDIADLEEGVEELTKNQNTLIEKDEEHDRLIAQNQADIQTLENNVVEELFNLSGRLIDQEADIAKNNASIEELYDFDNEVAERIGEIHAYTEEVNKTLENLITNSVKNTDNIDKNKADIDNNINHIYELAQQQDQHSSDIKTLKNNVEEGLLELSGHLIDQKADLTKDIKALESNVEEGLLDLSGRLLDQKADLTKDIKALESNVEEGLLDLSGRLLDQKADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 3 - protein, 644 aa, Moraxella catarrhalis
MKTMKLLPLKIAVTSALIIGLGAASTANAQQQLQTETFLPNFLSNDNYDLTDPFYHNMILGDTALLDKQDGSQPQLKFYSNDKDSVPDSLLFSKLLHEQQLNGFKKGDTIIPLDKDGKPVYQVDYKLDGKGKKQKRRQVYSVTTKTATDDDVNSAYSRGILGKVDDLDDEMNFLNHDITSLYDVTANQQDAIKDLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 4 - protein, 591 aa, Moraxella catarrhalis
MKTMKLLPLKIAVTSALIVGLGAASTANAQLVERFFPNIFLDKPLAKQHYHNVVVGDTSIVSDLQSNSDQLKFYSDDEGLVPDSLLFNKMLHEQLLNGFKEGDTIIPLDENGKPVYKVDYKLDGKEPRKVYSVTTKIATAEDVATSSYANGIQKDIDDLYDFDHQVTERLTQHGKTIYRNGERILANEESVQYLNKEVQNNIEHIYELAQQQDQHSSDIKTLESNVEKGLLELSGHLIDQKADLTKDIKTLESNVEEGLLDLSGRLIDQKADLTKDIKTLESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 5 - protein, 687 aa, Moraxella catarrhalis
MKTMKLLPLKIAVTSAMIIGLGAASTANAQQQQQQQQQQQSRTEIFFPNIFFNENHDELDDAYHNIILGDTALLDKQDGSQPQLKFYSNDKDSVPDSLLFSKLLHEQQLNGFKKGDTIIPLDKDGKPVYQVDYKLDGKGKKQKRRQVYSVTTKTATDDDVNSAYSRGILGKVDDLDDEMNFLNHDITSLYDVTANQQDAIKGLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 6 - protein, 683 aa, Moraxella catarrhalis
MKTMKLLPLKIAVTSAMIVGLGMASTANAQQQKSPKTETFLPNIFFNEYADDLDTLYHNMILGDTAITHDDQYKFYADDATEVPDSLFFNKILHDQLLYGFKEGDKIIPLDENGKPVYKLDKRLENGVQKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 7 - protein, 684 aa, Moraxella catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKEGGTIIPLDENGKPVYKLDSIVEQGKTKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 8 - protein, 684 aa, Moraxella catarrhalis
MKTMKLLPLKIAVTSAMIIGLGAASTANAQQQQKTKTEVFLPNLFDNDYYDLTDPLYHSMILGDTATLFDQQDNSKSQLKFYSNDKDSVPDSLLFSKLLHEQQLNGFKAGDTIIPLDKDGKPVYTQDTRTKDGKVETVYSVTTKIATQDDVEQSAYSRGIQGDIDDLYDINREVNEYLKATHDYNERQTEAIDALNKASSANTDRIDTAEERIDKNEYDIKALESNVGKDLLDLSGRLIAQKEDIDNNINHIYELAQQQDQHSSDIKTLKNNVEEGLLELSGHLIDQKADLTKDIKTLENNIEEGLLELSGHLIDQKADLTKDIKTLENNIEEGLLELSGHLIDQKADIAQNQANIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 9 - protein, 684 aa, Moraxella catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKKGDTIIPLDENGKPVYKLDSIVEQGKTKTVYSVTTKTATADDVNSAYSRGIQGDIDDLYEANKENVNRLIEHGDKIFANEESVQYLNREVQNNIENIYELVQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 10 - protein, 574 aa, Moraxella catarrhalis
MKTMKLLPLKIAVTSAMIIGLGAASTANAQLAEQFFPNIFSNHAPVKQHYHNVVVGDTSIVENLQDSDDTQLKFYSNDEYSVPDSLLFNKMLHEQQLNGFKKGDTIIPLDENGKPVYKVDYKLDGQEPRRVYSVTTKIATQDDVDNSPYSRGIQGDIDDLYEANKENVNRLIEHGDKIFANEESVQYLNKEVQNNIENIYELAQQQDQHSSDIKTLKKNVEEGLLELSGHLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 11 - protein, 678 aa, Moraxella catarrhalis
MKTMKLLPLKIAVTSALIVGLGAASTANAQQQPQTETFFPNIFFNENHDALDDVYHNMILGDTAITQDNQYKFYADAISEVPDSLLFNKILHDQQLNGFKEGDTIIPLDENGKPVYKLDEKVENGVKKSVYSVTTKTATRADVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKTLESNVEEGLLDLSGRLLDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 12 - protein, 678 aa, Moraxella catarrhalis
MKTMKLLPLKIAVTSAMIVGLGAASTANAQQQQQPRTETFFPNIFFNENHDALDDVYHNMILGDTAITQDNQYKFYADAISEVPDSLLFNKILHDQQLNGFKEGDTIIPLDENGKPVYKLDEKVENGVKKSVYSVTTKTATRADVEQSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKTLENNIEEGLLELSGHLIDQKADLTKDIKALESNVEEGLLDLSGRLLDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDTNKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 13 - protein, 587 aa, Moraxella catarrhalis
MKTMKLLPLKIAVTSALIVGLGAASTANAQLVERFFPNIFLDKPLAKQHYHNVVVGDTSIVSDLQSNSDQLKFYSDDEGLVPDSLLFNKMLHEQLLNGFKEGDTIIPLDENGKPVYKVDYKLDGKEPRKVYSVTTKIATAEDVATSSYANGIQKDIDDLYDFDHQVTERLTQHGKTIYRNGERILANEESVQYLNKEVQNNIEHIYELAQQQDQHSSDIKTLESNVEKGLLELSGHLIDQKADLTKDIKTLENNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDTNKASADTKFAATADAITKNGNAITKNAKSITDLGTKVDGFDSRVTALDTKVNALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 14 - protein, 678 aa, Moraxella catarrhalis; identical to SEQ ID NO: 12

SEQ ID NO: 15 - protein, 616 aa, Moraxella catarrhalis
MKTMKLLPLKIAVTSALIVGLGAASTANAQVRDKSLEDIEALLGKIDISKLEKEKKQQTELQKYLLLSQYANVLTMEELNKNVEKNTNSIEALGYEIGWLENDIADLEEGVEELTKNQNTLIEKDEEHDRLIAQNQADIKTLENNVVEELFNLSDRLIDQEADIAKNNASIEELYDFDNEVAERIGEIHAYTEEVNKTLEKLITNSVKNTDNIDKNKADIQALENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 16 - protein, 676 aa, Moraxella catarrhalis
MKTMKLLPLKIAVTSALIVGLGAASTANAQATETFLPNLFDNDYTETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKALESNVEEGLLDLSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 17 - protein, 629 aa, Moraxella catarrhalis
MKTMKLLPLKIAVTSALIVGLGAASTANAQNGTSTKLKNLKEYAQYLDNYAQYLDDDIDDLDKEVGELSQNIAKNQANIKDLNKKLSRDIDSLREDVYDNQYEIVNNQADIEKNQDDIKELENNVGKELLNLSGRLLDQKADIDNNINNIYELAQQQDQHSSDIKTLKKNVEEGLLELSGHLIDQKSDIAQNQTDIQDLATYNELQDQYAQKQTEAIDALNKASSENTQNIEDLAAYNELQDAYAKQQTEAIDALNKASSENTQNIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 18 - protein, 683 aa, Moraxella catarrhalis
MKTMKLLPLKIAVTSAMIVGLGMASTANAQQQRSPKTETFLPNIFFNEYADDLDTLYHNMILGDTAITHDDQYKFYADDATEVPDSLFFNKILHDQLLYGFKEGDKIIPLDENGKPVYKLDKRLDNGVQKTVYSVTTKTATADDVNSAYSRGIQGDIDDLYEANKENVNRLIEHGDKIFANEESVQYLNKEVQNNIENIHELAQQQDQHSSDIKTLKKNVEEGLLELSGHLIDQKTDIAQNQTDIQDLATYNELQDQYAQKQTEAIDALNKASSENTQNIAKNSNRIKALENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 19 - protein, 700 aa, Moraxella catarrhalis
MKTMKLLPLKIAVTSAMIVGLGAASTANAQAQSNRSLDQVQALLRGIDETKIKKEIQQSQQPELNKYLTFNQLANALNIEELNNNVQKNTQRLDSAATLYGDLSKTVPKSIKENKESIKENKESIKENKESIKENKESIKENKESIKENKESITTLTRKSFQNQVDIVRNNASIEDLYAYGQEVAKSIGEIHAYTEEVNKTLENLITNSVENTNNITKNKADIQALENNVVEELFNLSGRLIDQKADIDNNINNIYELAQQQDQHSSDIKTLKKNVEEGLLELSGHLIDQKADLTKDIKTLESNVEEGLLDLSGRLLDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTVIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 20 - protein, 676 aa, Moraxella catarrhalis
MKTMKLLPLKIAVTSALIVGLGAASTANAQATETFLPNLFDNDYIETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKTLENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 21 - protein, 678 aa, Moraxella catarrhalis
MKTMKLLPLKIAVTSAMIIGLGAASTANAQLAEQFFPNIFSNHAPVKQHYHNVVVGDTSIVENLQDSDDTQLKFYSNDEYSVPDSLLFNKMLHEQQLNGFKKGDTIIPLDENGKPVYKVDYKLDGQEPRRVYSVTTKIATQDDVDNSPYSRGIQGDIDDLYEANKENVNRLIEHGDKIFANEESVQYLNKEVQNNIENIYELAQQQDQHSSDIKTLKKNVEEGLLELSGRLIDQKADIAQNQANIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 22 - protein, 613 aa, Moraxella catarrhalis; identical to SEQ ID NO: 2

SEQ ID NO: 23 - protein, 589 aa, Moraxella catarrhalis
MKTMKLLPLKIAVTSALIVGLGAASTANAQATNKDITLEDVLKSIEEIDPYELRDYIEYPTAIERFLLLSQYGNTLTLEEFDNDIELLDQDVEDLEESVTELAKNQNSLIEQGEAIKEDLQGLADFVERQEDKILQNETSIKKNTQRNLVNGFEIEKNKDAIAKNNESIEDLYDFGHEVAKSIGEIHAHNEAQNETLKDLITNSVKNTDNITKNKADIQALESNVEKGLLELSGHLIDQKADIDNNINNIHELAQQQDQHSSDIKTLKKNVEEGLLELSGHLIDQKSDIAQNQANIQDLATYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 24 - protein, 684 aa, Moraxella catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKEGDTIIPLDENGKPVYKLDSIVEQGKTKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 25 - protein, 650 aa, Moraxella catarrhalis
MKTMKLLPLKIAVTSALIVGLGAASTANAQQQQKTKTEVFLPNLFYNDYIEETDLLYHNMILGDTAALVDRQNYSNSQLKFYSNDEESVPDSLLFSKMLNNQQLNGFKAGDIIIPVDANGQVIYQKDTRVEGGKTRTVLSVTTKIATQQDVDSAYSRGIQGKVNDLDDEMNFLNHDITSLYDVTANQQDDIKGLKKGVKDLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 26 - protein, 616 aa, Moraxella catarrhalis
MKTMKLLPLKIAVTSALIVGLGAVSTTNAQAQSRSLDQIQTKLADLAGKIAAGKNGGGQNNQNNQNDINKYLFLSQYANILTMEELNNNVVKNSSSIETLETDFGWLENDVADLEDGVEELTKNQNTLIEKDEEHDRLIAQNQADIQTLENNVVEELFNLSDRLIDQKADIAKNQADIAQNNESIEELYDFDNEVAEKIGEIHAYTEEVNKTLQDLITNSVKNTDNIDKNKADIDNNINHIYELAQQQDQHSSDIKTLKNNVEEGLLELSGHLIDQKADLTKDIKTLENNVEEGLLDLSGRLIDQKADIAKNQADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 27 - protein, 668 aa, Moraxella catarrhalis
MKTMKLLPLKIAVTSALIVGLGTASTANAQVASPANQKIQQKIKKVRKELRQDIKSLRNDIDSNTADIGSLNDDVADNQDDILDNQADIAKNQDDIEKNQADIKELDKEVGVLSREIGSLNDDIADNYTDIIDNYTDIIDNQANIAKNQDDIEKNQADIKELDKEVGVLSREIGSLNDDVADNQDDIAKNQADIQTLENNVEEGLLELSGHLLDQKADIDNNINNIYELAQQQDQHSSDIKTLKKNVEEGLLELSGHLIDQKTDIAQNQANIQDLATYNELQDQYAQEQTEAIDALNKASSENTQNIAKNSNRIKALESNVEEGLLELSGHLIDQKADLTKDIKALESNVEEGLLELSGHLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKVSADTKFAATADAITKNGNAITKNAKSITDLGTKVDAFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 28 - protein, 674 aa, Moraxella catarrhalis
MKTMKLLPLKIAVTSAMIVGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVEEGLLELSGHLIDQKADLTKDIKALESNVEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 29 - protein, 613 aa, Moraxella catarrhalis
MKTMKLLPLKIAVTSAMIIGLGAASTANAQSRDRSLEDIQDSISKLVQDDIDTLKQDQQKMNKYLLLNQLANTLITDELNNNVIKNTNSIEALGDEIGWLENDIADLEEGVEELTKNQNTLIEKDEEHDRLIAQNQADIQTLENNVVEELFNLSGRLIDQEADIAKNNASIEELYDFDNEVAERIGEIHAYTEEVNKTLENLITNSVKNTDNIDKNKADIDNNINHIYELAQQQDQHSSDIKTLKNNVEEGLLELSGHLIDQKADLTKDIKALESNVEEGLLDLSGRLLDQKADLTKDIKALESNVEEGLLDLSGRLLDQKADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 30 - protein, 576 aa, Moraxella catarrhalis
MKTMKLLPLKIAVTSAMIVGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVEEGLLELSGHLIDQKADLTKDIKALESNVEEGLLELSGHLIDQKADLTKDIKALESNVEEGLLDLSGRLLDQKADIAKNQADIAQNQTDIQDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 31 - protein, 684 aa, Moraxella catarrhalis; identical to SEQ ID NO: 24

SEQ ID NO: 32 - protein, 686 aa, Moraxella catarrhalis
MKTMKLLPLKIAVTSAMIIGLGAASTANAQATETFLPNLFDNDYTETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 33 - protein, 630 aa, Moraxella catarrhalis; identical to SEQ ID NO: 1

SEQ ID NO: 34 - protein, 616 aa, Moraxella catarrhalis
MKTMKLLPLKIAVTSALIVGLGAASTANAQAQDRSLEQIQDKLANLVEKIEQAKSQNGQSQKDINQYLLLSQYANVLTMEELNNNVVKNSSSIETLDNDIAWLNDDLIDLDKEVGVLSRDIGSLHDDVAQNQADIKTLKNNVVEELFNLSDRLIDQEADIAQNNESIEDLYDFGREVAESIGEIHAHNEAQNETLKDLITNSVKNTDNITKNKADIQALENDVGKELLNLSGRLIDQKADIDNNINHIYELAQQQDQHSSDIKTLKNNVEEGLLELSGHLIDQKADLTKDIKALESNVEEGLLDLSGRLLDQKADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 35 - protein, 614 aa, Moraxella catarrhalis
MKTMKLLPLKIAVTSAMIIGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVGKDLLDLSGRLIAQKEDIDNNINHIYELAQQQDQHSSDIKTLKNNVEEGLLELSGHLIDQKADLTKDIKTLESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 36 - protein, 679 aa, Moraxella catarrhalis
MKTMKLPPLKIAVTSAMIIGLGAASTANAQTTETFLPNLFDNDYTETTDPLYHGMILGDTAITQDTQYKFYAENGNEVPDSLFFNKILHDQLLNGFKAGDTIIPLDENGKPVYKLDERTENGVKRKVYSVTTKTATQADVEQSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEECLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 37 - protein, 724 aa, Moraxella catarrhalis
MKTMKLLPLKIAVTSALIVGLGAASTANAQETLEEVLESIKQINEQDLQDDIGYNSALDRYLVLSQYGNLLIAKELNENVEKNSNSIAKNSNSIADLEADVGYLAENQNTLIEQNETINQELEGITHELESFIAYAHAQDQKNLVNEFEIEKNKDAIAKNNESIEDLYDFGHEVAESIGEIHAYTEEVNKTLENLITNSVKNTDNITKNKADIQALESNVEKELLNLSGRLIDQKADIDNNINHIYELAQQQDQHSSDIKTLKKNVEEGLLELSGHLIDQKSDIAQNQTDIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 38 - protein, 611 aa, Moraxella catarrhalis
MKTMKLLPLKIAVTSALIVGLGAASTANAQAQARDRSLEDIQALIGNIDVDKIRSQKQKNPEIFQYLLLNQLSNTLITDELNNNVIKNTNSIETLDNDIAWLNDDLIDLDKEVGVLSRDIGSLHDDVAQNQADIKTLENNVVEELFNLSDRLIDQEAEIAQNNESIEDLYDFGREVAESIGEIHAHNEAQNETLKDLITNSVKNTDNIDKNKADIQALENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 39 - protein, 894 aa, Moraxella catarrhalis
MNKIYKVKKNAAGHSVACSEFAKGHTKKAVLGSLLIVGALGMATTASAQTGSTNAANGNIISGVGAYVGGGVINQAKGNYPTVGGGFDNRATGNYSVISGGFDNQAKGEHSTIAGGESNQATGRNSTVAGGSNNQAVGTNSTVAGGSNNQAKGANSFAAGVGNQANTDNAVALGKNNTINGNNSAAIGSENTVNENQKNVFILGSNTTNAQSGSVLLGHETSGKEATAVSRARVNGLTLKNFSGVSKADNGTVSVGSQGKERQIVHVGAGQISDDSTDAVNGSQLYALATAVDDNQYDIEINQDNIKDLQKEVKGLDKEVGVLSRDIGSLHDDVADNQADIAKNKADIKELDKEMNVLSRDIVSLNDDVADNQADIAKNQADIKTLENNVEEGLLDLSGRLIDQKADIDNNINHIYELAQQQDQHSSDIKTLKKNVEEGLLELSGHLIDQKADIAQNQTDIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIQDLAAYNELQDAYAKQQTEAIDALNKASSENTQNIQDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 40 - protein, 877 aa, Moraxella catarrhalis
MNKIYKVKKNAAGHSVACSEFAKGHTKKAVLGSLLIVGALGMATTASAQLVSTTQPNDYRSSTTGNNHLGSSWSIIGAGHDNIVYRSASNSGILSGYKNRVNGSTSAIVGGYDNETRGKYTFVGGGYKNLAEGHQSAIGGGYANWAEGDNATIAGGFENFAAGNQSAIGGGYANLAEGDDATIAGGFENRAEGNQSAIGGGYANFAAGDYTFVGGGYENRAEGNQSAIGGGYANLAEGDNATIAGGFENRAKGINSVVSGGYANQATGESSTIAGGFENRAEGIDSVVSGGYANQANGAQSTVAGGYNNQATGESSTIAGGSNNQATGTGSFAAGVENKANADNAVALGKNNIINGDNSAAIGSNNTVKKGQKDVFILGSNTSGAQSNSVLLGNETTGKKATAVENATVGDLSLTGFAGVSKANSGTVSVGSEGKERQIVHVGAGRISNDSTDAVNGSQLYALAAAVDDNQYDIEKNQDDIKELKRGVKELDKEMNVLSRDIVSLNDDVAQNQSDIKTLKNNVEEGLLELSGHLIDQKADLTKDIKALENNVEEGLLDLSGRLLDQKADIAKNQADIAQNQTDIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 41 - protein, 861 aa, Moraxella catarrhalis
VNKIYKVKKNVAGHSVACSEFAKGHTKKAVLGSLLIVGALGMATTASAQQNNKGFGAYIGVENSGNLATGQYSVAVGGLGNEATGQYSVINGGYFNIADGVHSIIGGGKDNKAKGNYSTIGGGDSNKAEGNYSTIAGGRNNEATGEGSFAAGIDNKANAKNAVALGNKNSIEGTNSVAIGSNNTVAQDNTFILGSNTTGVQRNSVLLGNNTAGQEATIVEKAEVGGVNLTGFAGVSKTSNGTVSVGSKNNERQIVNVGAGRISKDSTDAVNGSQLYALATAVDDSQYDIEKNQDDIEKNQDDIKELKRGVKDLKKGVKGLDKEVKDLNKKVGVLDRDIGSLQDDVADNQADIANNQADIKDLDKEVGVLSRDIGSLHDDVAQNQSDIKTLKNNVEEGLLELSGHLLDQKADIDNNINNIYELAQQQDQHSSDIKTLKKNVEEGLLELSGHLIDQKADIAQNQTDIQDLATYNELQDQYAQKQTEAIDALNKASSENTQNIAKNSNRIKALESNVEEGLLELSGHLIDQKADLTKDIKALESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 42 - protein, 875 aa, Moraxella catarrhalis
VNKIYKVKKNVAGHSVACSEFAKGHTKKAVLGSLLIAGALGMATTASAQVVGNTTEGLGARVNGGDGNEAKGTYPTIGGGFFNIATGNYSTISGGNTNEAEGNYSTIGGGDTNAASGNNSTVVGGYNNTAKGNYSTVAGGANNQANSIDSTIVGGRKNRAKGKSSTVSGGYDNLAGGKSSAIGGGEFNSAIGSRSTVAGGELNLASGDNATVSGGKQNQAIGEYSTVAGGELNLASGDNATVSGGKQNQAEGTDSTIAGGRNNQAIGEGSFAAGIDNKANTENAVAVGKKNIINGDNSAAIGSNNTVKKGQTGVFILGSNTKGAQSNSVLLGNETTGKKATAVSSATVNGLTLENFAGVSQVGNGTVSVGSKGKERQIVNVGAGKISATSTDAVNGSQLHALATAVAYNYTDIIDNQADIAKNQDDIDDLNKKVGVLDRDIGSLQDDVADNQADIANNQADIKDLDDEVGVLSRDIGSLHDDVAQNQSDIKTLKNNVEEGLLDLSGRLLDQKADIDNNINNIYELAQQQDQHSSDIKTLKKNVEEGLLELSGHLIDQKSDIAKNQADIAQNQTDIHDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 43 - protein, 876 aa, Moraxella catarrhalis
VNKIYKVKKNAAGHLVACSEFAKGHTKKAVLGSLLIVGILGMATTASAQDRAGNQRNGAHSAIGGGSINIADGDYSTIGGGHINTTKGNYSTIGGGNENKTTGNYSTIGGGYGNEAEGKHSTIGGGDENKAKGDYSTIGGGDDNEATGTHSTIGGGDDNKATGDYSIIGGGYGNRATAEDATVSGGGYNQAAGKSSTVSGGYDNLAEGESSAIGGGELNSTIGSHSTVAGGFLNGAVGDNATVSGGEQNQAIGKYSTVSGGYGNQATGIGSFAAGIENQANTENAVAVGKKNIINGDNSAAIGSNNTVEKGQKDVFILGSNTKDAQSNSVLLGNETTGKAATVVDSANVGGLGLTGFAGVSKVGNGTVSVGSQGKERQIVNVGAGEISATSTDAVNGSQLHALATAVDNNQYDIINNQGDIERNQDDIKDLQKEVKGLDNEVGELSRDINSLHDVTDNQQDDIKELKRGVKELDKEMNVLSRDIVSLNDDVADNQADIAKNQADIKTLESNVEEGLLDLSGRLLDQKADIANNVNHIYELAQQQDQHSSDIKTLKKNVEEGLLELSGHLIDQKSDIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 44 - protein, 655 aa, Moraxella catarrhalis
MKTMKLLPLKIAVTSALIVGLGAASTANAQKKASSQELNQHIRVLNKYIRDFNDDIQELDYDIQDLNDDIKGLDKKLSRDINSLRDEVTDNYTDIIDNQVDIERNQADIKDLKRGVKGLNKEVGVLDRDIGSLQDDVADNQADIANNQDDIKDLDDEVGVLSRDIGSLHDDVAQNQSDIKTLESNVEEGLLELSGHLIDQKADIDNNINHIYELAQQQDQHSSDIKTLKKNVEEGLLELSGHLIDQKSDIAQNQTDIQDLATYNELQDQYAQKQTEAIDALNKASSENTQNIQDLAAYNELQDAYAKQQTEAIDALNKASSENTQNIEDLAAYNELQDQYAQKQTEAIDALNKASSENTQNIQDLAAYNELQDAYAKQQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 45 - protein, 591 aa, Moraxella catarrhalis; identical to SEQ ID NO: 4

SEQ ID NO: 46 - protein, 687 aa, Moraxella catarrhalis; identical to SEQ ID NO: 5

SEQ ID NO: 47 - protein, 863 aa, Moraxella catarrhalis
MNKIYKVKKNAAGHLVACSEFAKGHTKKAVLGSLLIVGILGMATTASAQKVGKATNKISGGDNNTANGTYLTIGGGDYNKTKGRYSTIGGGLFNEATNEYSTIGSGGYNKAKGRYSTIGGGGYNEATNQYSTIGGGDNNTAKGRYSTIGGGGYNEATIENSTVGGGGYNQAKGRNSTVAGGYNNEATGTDSTIAGGRKNQATGKGSFAAGIDNKANADNAVALGNKNTIEGENSVAIGSNNTVKKGQQNVFILGSNTDTTNAQNGSVLLGHNTAGKAATIVNSAEVGGLSLTGFAGASKTGNGTVSVGKKGKERQIVHVGAGEISDTSTDAVNGSQLHALATVVAQNKADIKDLDDEVGLLGEEINSLEGEIFNNQDAIAKNQADIKTLESNVEEGLLDLSGRLLDQKADIDNNINNIYELAQQQDQHSSDIKTLKNNVEEGLLDLSGRLIDQKADLTKDIKALESNVEEGLLDLSGRLIDQKADIAKNQADIAQNQTDIQDLAAYNELQDAYAKQQTEAIDALNKASSANTDRIATAELGIAENKKDAQIAKAQANENKDGIAKNQADIQLHDKKITNLGILHSMVARAVGNNTQGVATNKADIAKNQADIANNIKNIYELAQQQDQHSSDIKTLAKVSAANTDRIAKNKAEADASFETLTKNQNTLIEQGEALVEQNKAINQELEGFAAHADVQDKQILQNQADITTNKTAIEQNINRTVANGFEIEKNKAGIATNKQELILQNDRLNRINETNNHQDQKIDQLGYALKEQGQHFNNRISAVERQTAGGIANAIAIATLPSPSRAGEHHVLFGSGYHNGQAAVSLGAAGLSDTGKSTYKIGLSWSDAGGLSGGVGGSYRWK

SEQ ID NO: 48 - protein, 815 aa, Moraxella catarrhalis
QKVGKATNKISGGDNNTANGTYLTIGGGDYNKTKGRYSTIGGGLFNEATNEYSTIGSGGYNKAKGRYSTIGGGGYNEATNQYSTIGGGDNNTAKGRYSTIGGGGYNEATIENSTVGGGGYNQAKGRNSTVAGGYNNEATGTDSTIAGGRKNQATGKGSFAAGIDNKANADNAVALGNKNTIEGENSVAIGSNNTVKKGQQNVFILGSNTDTTNAQNGSVLLGHNTAGKAATIVNSAEVGGLSLTGFAGASKTGNGTVSVGKKGKERQIVHVGAGEISDTSTDAVNGSQLHALATVVAQNKADIKDLDDEVGLLGEEINSLEGEIFNNQDAIAKNQADIKTLESNVEEGLLDLSGRLLDQKADIDNNINNIYELAQQQDQHSSDIKTLKNNVEEGLLDLSGRLIDQKADLTKDIKALESNVEEGLLDLSGRLIDQKADIAKNQADIAQNQTDIQDLAAYNELQDAYAKQQTEAIDALNKASSANTDRIATAELGIAENKKDAQIAKAQANENKDGIAKNQADIQLHDKKITNLGILHSMVARAVGNNTQGVATNKADIAKNQADIANNIKNIYELAQQQDQHSSDIKTLAKVSAANTDRIAKNKAEADASFETLTKNQNTLIEQGEALVEQNKAINQELEGFAAHADVQDKQILQNQADITTNKTAIEQNINRTVANGFEIEKNKAGIATNKQELILQNDRLNRINETNNHQDQKIDQLGYALKEQGQHFNNRISAVERQTAGGIANAIAIATLPSPSRAGEHHVLFGSGYHNGQAAVSLGAAGLSDTGKSTYKIGLSWSDAGGLSGGVGGSYRWK

SEQ ID NO: 49 - protein, 876 aa, Moraxella catarrhalis
MNKIYKVKKNAAGHLVACSEFAKGHTKKAVLGSLLIVGILGMATTASAQDRAGNQRNGAHSAIGGGSINIADGDYSTIGGGHINTTKGNYSTIGGGNENKTTGNYSTIGGGYGNEAEGKHSTIGGGDENKAKGDYSTIGGGDDNEATGTHSTIGGGDDNKATGDYSIIGGGYGNRATAEDATVSGGGYNQAAGKSSTVSGGYDNLAEGESSAIGGGELNSTIGSHSTVAGGFLNGAVGDNATVSGGEQNQAIGKYSTVSGGYGNQATGIGSFAAGIENQANTENAVAVGKKNIINGDNSAAIGSNNTVEKGQKDVFILGSNTKDAQSNSVLLGNETTGKAATVVDSANVGGLGLTGFAGVSKVGNGTVSVGSQGKERQIVNVGAGEISATSTDAVNGSQLHALATAVDNNQYDIINNQGDIERNQDDIKDLQKEVKGLDNEVGELSRDINSLHDVTDNQQDDIKELKRGVKELDKEMNVLSRDIVSLNDDVADNQADIAKNQADIKTLESNVEEGLLDLSGRLLDQKADIANNVNHIYELAQQQDQHSSDIKTLKKNVEEGLLELSGHLIDQKSDIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 50 - protein, 828 aa, Moraxella catarrhalis
QDRAGNQRNGAHSAIGGGSINIADGDYSTIGGGHINTTKGNYSTIGGGNENKTTGNYSTIGGGYGNEAEGKHSTIGGGDENKAKGDYSTIGGGDDNEATGTHSTIGGGDDNKATGDYSIIGGGYGNRATAEDATVSGGGYNQAAGKSSTVSGGYDNLAEGESSAIGGGELNSTIGSHSTVAGGFLNGAVGDNATVSGGEQNQAIGKYSTVSGGYGNQATGIGSFAAGIENQANTENAVAVGKKNIINGDNSAAIGSNNTVEKGQKDVFILGSNTKDAQSNSVLLGNETTGKAATVVDSANVGGLGLTGFAGVSKVGNGTVSVGSQGKERQIVNVGAGEISATSTDAVNGSQLHALATAVDNNQYDIINNQGDIERNQDDIKDLQKEVKGLDNEVGELSRDINSLHDVTDNQQDDIKELKRGVKELDKEMNVLSRDIVSLNDDVADNQADIAKNQADIKTLESNVEEGLLDLSGRLLDQKADIANNVNHIYELAQQQDQHSSDIKTLKKNVEEGLLELSGHLIDQKSDIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 51 - protein, 786 aa, Moraxella catarrhalis
MKTMKLLPLKIAVTSAMIVGLGAASNANAQTSNSGTNQIVEEFLPKLTQALNDPQYQHEFEDALDQYHNMVVGNTSFVSTVDVANQTVGNTALKFYSDSENTLPSSMLFDQILTNQRLNGFKEGQPFIPVDDNGNPITQVDDVLESNGQTKKVRSVTTRIATAEDVTKSPYAQGIQKDIDDIYEFNHEVVDNIATLNRATEILTEAGNHHDELIRKNKADIQVSNGKIESLKTLYETTVRTVGNNESNIKTNKDAIAKNKANIKTLAQAQLESVTFLGEQIQENKDDISAVTGKAFKNQIDIQRNNDSIEDLYEANNDNVERLVEHDRAIEILAEAGNHHDTLIRKNKADIQDLAAHNELQTEAIDALNKASSANTKRITTAELGIAENKKDALIAKAQADKNKNDIQDLAKVQRAGVEVMAELNKNIAGTQTDIANNKITLAQHAKKIDENQQAIEAKLGGKADLQKLSELKTSVDKNQAGIQLHDQKINNLGILHTMVARAVGANKTAIDQNKADIAENKEDIQNNINNIYELAQQQDQHSSDIKTLAKASSANTDNIAKNKADADASFKTLTKNQNTLIEKDKAQDALITANKADADASFKTLTKNQNTLIEKDKAQDKLITANKADADAKFTGVQTDIANNKTTLAQHAKKINDNQQAIEAKLGGKADAQALNNLNDKVNGFDGRISALDTKVNAFDGRITALDSKVENGMAAQAALSGLFQPYSVGKFNATAALGGYGSKSAVAIGAGYRVNPNLAIKAGAAINTSGDKKGSYNIGVNYEF

SEQ ID NO: 52 - protein, 757 aa, Moraxella catarrhalis
QTSNSGTNQIVEEFLPKLTQALNDPQYQHEFEDALDQYHNMVVGNTSFVSTVDVANQTVGNTALKFYSDSENTLPSSMLFDQILTNQRLNGFKEGQPFIPVDDNGNPITQVDDVLESNGQTKKVRSVTTRIATAEDVTKSPYAQGIQKDIDDIYEFNHEVVDNIATLNRATEILTEAGNHHDELIRKNKADIQVSNGKIESLKTLYETTVRTVGNNESNIKTNKDAIAKNKANIKTLAQAQLESVTFLGEQIQENKDDISAVTGKAFKNQIDIQRNNDSIEDLYEANNDNVERLVEHDRAIEILAEAGNHHDTLIRKNKADIQDLAAHNELQTEAIDALNKASSANTKRITTAELGIAENKKDALIAKAQADKNKNDIQDLAKVQRAGVEVMAELNKNIAGTQTDIANNKITLAQHAKKIDENQQAIEAKLGGKADLQKLSELKTSVDKNQAGIQLHDQKINNLGILHTMVARAVGANKTAIDQNKADIAENKEDIQNNINNIYELAQQQDQHSSDIKTLAKASSANTDNIAKNKADADASFKTLTKNQNTLIEKDKAQDALITANKADADASFKTLTKNQNTLIEKDKAQDKLITANKADADAKFTGVQTDIANNKTTLAQHAKKINDNQQAIEAKLGGKADAQALNNLNDKVNGFDGRISALDTKVNAFDGRITALDSKVENGMAAQAALSGLFQPYSVGKFNATAALGGYGSKSAVAIGAGYRVNPNLAIKAGAAINTSGDKKGSYNIGVNYEF

SEQ ID NO: 53 - protein, 511 aa, Moraxella catarrhalis
QAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTK

SEQ ID NO: 54 - protein, 510 aa, Moraxella catarrhalis
AKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTK

SEQ ID NO: 55 - protein, 490 aa, Moraxella catarrhalis
QAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKS

SEQ ID NO: 56 - protein, 535 aa, Moraxella catarrhalis
QAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAA

SEQ ID NO: 57 - protein, 534 aa, Moraxella catarrhalis
AKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAA

SEQ ID NO: 58 - DNA, 1563 bp, Artificial Sequence
  DNA Sequences for the MC-001 - MC-011 constructs
atgcaggccaaaaatgatattaccctggaagatctgccgtatctgatcaaaaaaatcgatcagaacgaactggaagccgatattggtgatattaccgcactggaaaaatatctggcactgagccagtatggaaatattctggccctggaagaactgaataaagctctggaagagctggatgaagatgtgggttggaatcagaatgatatcgccaatctggaagatgatgttgaaaccctgaccaaaaatcagaatgcactggcagaacagggtgaagcaattaaagaagatctgcagggtctggcagattttgttgaaggtcaggaaggcaaaattctgcagaacgaaaccagcatcaaaaaaaacacccagcgtaatctggtgaatggctttgaaattgaaaaaaacaaagatgccattgccaaaaacaacgaaagcattgaagatctgtatgattttggtcatgaagttgccgaaagcattggtgaaattcatgcacataacgaagcacagaatgaaaccctgaaaggtctgattaccaacagcatcgaaaataccaataacattaccaaaaacaaagcagatattcaggcgctggaaaataatgttgtggaagaactgtttaatctgagcggtcgtctgattgatcagaaagccgatatcgataataacattaacaacatttatgaactggcacagcagcaggatcagcatagcagcgatatcaaaaccctgaaaaaaaacgttgaagaaggtctgctggaactgtctggtcacctgatcgatcagaaaactgatattgcccagaatcaggcaaatattcaggatctggccacctataatgaactgcaggatcagtatgcacagaaacagaccgaagcaattgatgccctgaataaagcgagcagcgaaaacacccagaatatcgaagatctggcagcatacaacgaactgcaggatgcctatgcaaaacagcagactgaagccatcgacgcactgaacaaggcaagctctgaaaacacgcagaacattgaagatctggctgcctataatgaattacaggatgcgtatgccaaacagcagaccgaagcgattgatgcgctgaacaaagcctcttctgaaaatacacagaatatcgccaaaaatcaggccgatattgccaacaatatcaataatatctatgaactggcccagcagcaggatcagcactcttctgatatcaaaacactggcaaaagcaagcgcagcaaataccgatcgtattgcgaaaaacaaagccgatgcagatgcaagctttgaaacactgacgaaaaaccagaacaccctgattgaaaaagataaagaacatgataaactgatcaccgccaataaaaccgcaattgatgcaaataaagccagcgcagataccaaatttgcagcaaccgcagatgcaattaccaaaaatggcaatgccatcaccaaaaatgccaaaagcattaccgatctgggcaccaaagttgatggttttgatagccgtgtgaccgcactggataccaaagcaagccatcatcatcaccaccactaa

SEQ ID NO: 59 - protein, 520 aa, Artificial Sequence
  Protein Sequences for the MC-001 - MC-011 constructs
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKASHHHHHH

SEQ ID NO: 60 - DNA, 1539 bp, Artificial Sequence
  DNA Sequences for the MC-001 - MC-011 constructs
atgcaggccaaaaatgatattaccctggaagatctgccgtatctgatcaaaaaaatcgatcagaacgaactggaagccgatattggtgatattaccgcactggaaaaatatctggcactgagccagtatggaaatattctggccctggaagaactgaataaagctctggaagagctggatgaagatgtgggttggaatcagaatgatatcgccaatctggaagatgatgttgaaaccctgaccaaaaatcagaatgcactggcagaacagggtgaagcaattaaagaagatctgcagggtctggcagattttgttgaaggtcaggaaggcaaaattctgcagaacgaaaccagcatcaaaaaaaacacccagcgtaatctggtgaatggctttgaaattgaaaaaaacaaagatgccattgccaaaaacaacgaaagcattgaagatctgtatgattttggtcatgaagttgccgaaagcattggtgaaattcatgcacataacgaagcacagaatgaaaccctgaaaggtctgattaccaacagcatcgaaaataccaataacattaccaaaaacaaagcagatattcaggcgctggaaaataatgttgtggaagaactgtttaatctgagcggtcgtctgattgatcagaaagccgatatcgataataacattaacaacatttatgaactggcacagcagcaggatcagcatagcagcgatatcaaaaccctgaaaaaaaacgttgaagaaggtctgctggaactgtctggtcacctgatcgatcagaaaactgatattgcccagaatcaggcaaatattcaggatctggccacctataatgaactgcaggatcagtatgcacagaaacagaccgaagcaattgatgccctgaataaagcgagcagcgaaaacacccagaatatcgaagatctggcagcatacaacgaactgcaggatgcctatgcaaaacagcagactgaagccatcgacgcactgaacaaggcaagctctgaaaacacgcagaacattgaagatctggctgcctataatgaattacaggatgcgtatgccaaacagcagaccgaagcgattgatgcgctgaacaaagcctcttctgaaaatacacagaatatcgccaaaaatcaggccgatattgccaacaatatcaataatatctatgaactggcccagcagcaggatcagcactcttctgatatcaaaacactggcaaaagcaagcgcagcaaataccgatcgtattgcgaaaaacaaagccgatgcagatgcaagctttgaaacactgacgaaaaaccagaacaccctgattgaaaaagataaagaacatgataaactgatcaccgccaataaaaccgcaattgatgcaaataaagccagcgcagataccaaatttgcagcaaccgcagatgcaattaccaaaaatggcaatgccatcaccaaaaatgccaaaagcattaccgatctgggcaccaaagttgatggttttgatagccgtgtgaccgcactggataccaaataa

SEQ ID NO: 61 - protein, 512 aa, Artificial Sequence
  Protein Sequences for the MC-001 - MC-011 constructs
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTK

SEQ ID NO: 62 - DNA, 1542 bp, Artificial Sequence
  DNA Sequences for the MC-001 - MC-011 constructs
atgcaggccaaaaatgatattaccctggaagatctgccgtatctgatcaaaaaaatcgatcagaacgaactggaagccgatattggtgatattaccgcactggaaaaatatctggcactgagccagtatggaaatattctggccctggaagaactgaataaagctctggaagagctggatgaagatgtgggttggaatcagaatgatatcgccaatctggaagatgatgttgaaaccctgaccaaaaatcagaatgcactggcagaacagggtgaagcaattaaagaagatctgcagggtctggcagattttgttgaaggtcaggaaggcaaaattctgcagaacgaaaccagcatcaaaaaaaacacccagcgtaatctggtgaatggctttgaaattgaaaaaaacaaagatgccattgccaaaaacaacgaaagcattgaagatctgtatgattttggtcatgaagttgccgaaagcattggtgaaattcatgcacataacgaagcacagaatgaaaccctgaaaggtctgattaccaacagcatcgaaaataccaataacattaccaaaaacaaagcagatattcaggcgctggaaaataatgttgtggaagaactgtttaatctgagcggtcgtctgattgatcagaaagccgatatcgataataacattaacaacatttatgaactggcacagcagcaggatcagcatagcagcgatatcaaaaccctgaaaaaaaacgttgaagaaggtctgctggaactgtctggtcacctgatcgatcagaaaactgatattgcccagaatcaggcaaatattcaggatctggccacctataatgaactgcaggatcagtatgcacagaaacagaccgaagcaattgatgccctgaataaagcgagcagcgaaaacacccagaatatcgaagatctggcagcatacaacgaactgcaggatgcctatgcaaaacagcagactgaagccatcgacgcactgaacaaggcaagctctgaaaacacgcagaacattgaagatctggctgcctataatgaattacaggatgcgtatgccaaacagcagaccgaagcgattgatgcgctgaacaaagcctcttctgaaaatacacagaatatcgccaaaaatcaggccgatattgccaacaatatcaataatatctatgaactggcccagcagcaggatcagcactcttctgatatcaaaacactggcaaaagcaagcgcagcaaataccgatcgtattgcgaaaaacaaagccgatgcagatgcaagctttgaaacactgacgaaaaaccagaacaccctgattgaaaaagataaagaacatgataaactgatcaccgccaataaaaccgcaattgatgcaaataaagccagcgcagataccaaatttgcagcaaccgcagatgcaattaccaaaaatggcaatgccatcaccaaaaatgccaaaagcattaccgatctgggcaccaaagttgatggttttgatagccgtgtgaccgcactggataccaaacactaa

SEQ ID NO: 63 - protein, 513 aa, Artificial Sequence
  Protein Sequences for the MC-001 - MC-011 constructs
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKH

SEQ ID NO: 64 - DNA, 1545 bp, Artificial Sequence
  DNA Sequences for the MC-001 - MC-011 constructs
atgcaggccaaaaatgatattaccctggaagatctgccgtatctgatcaaaaaaatcgatcagaacgaactggaagccgatattggtgatattaccgcactggaaaaatatctggcactgagccagtatggaaatattctggccctggaagaactgaataaagctctggaagagctggatgaagatgtgggttggaatcagaatgatatcgccaatctggaagatgatgttgaaaccctgaccaaaaatcagaatgcactggcagaacagggtgaagcaattaaagaagatctgcagggtctggcagattttgttgaaggtcaggaaggcaaaattctgcagaacgaaaccagcatcaaaaaaaacacccagcgtaatctggtgaatggctttgaaattgaaaaaaacaaagatgccattgccaaaaacaacgaaagcattgaagatctgtatgattttggtcatgaagttgccgaaagcattggtgaaattcatgcacataacgaagcacagaatgaaaccctgaaaggtctgattaccaacagcatcgaaaataccaataacattaccaaaaacaaagcagatattcaggcgctggaaaataatgttgtggaagaactgtttaatctgagcggtcgtctgattgatcagaaagccgatatcgataataacattaacaacatttatgaactggcacagcagcaggatcagcatagcagcgatatcaaaaccctgaaaaaaaacgttgaagaaggtctgctggaactgtctggtcacctgatcgatcagaaaactgatattgcccagaatcaggcaaatattcaggatctggccacctataatgaactgcaggatcagtatgcacagaaacagaccgaagcaattgatgccctgaataaagcgagcagcgaaaacacccagaatatcgaagatctggcagcatacaacgaactgcaggatgcctatgcaaaacagcagactgaagccatcgacgcactgaacaaggcaagctctgaaaacacgcagaacattgaagatctggctgcctataatgaattacaggatgcgtatgccaaacagcagaccgaagcgattgatgcgctgaacaaagcctcttctgaaaatacacagaatatcgccaaaaatcaggccgatattgccaacaatatcaataatatctatgaactggcccagcagcaggatcagcactcttctgatatcaaaacactggcaaaagcaagcgcagcaaataccgatcgtattgcgaaaaacaaagccgatgcagatgcaagctttgaaacactgacgaaaaaccagaacaccctgattgaaaaagataaagaacatgataaactgatcaccgccaataaaaccgcaattgatgcaaataaagccagcgcagataccaaatttgcagcaaccgcagatgcaattaccaaaaatggcaatgccatcaccaaaaatgccaaaagcattaccgatctgggcaccaaagttgatggttttgatagccgtgtgaccgcactggataccaaacatcattaa

SEQ ID NO: 65 - protein, 514 aa, Artificial Sequence
  Protein Sequences for the MC-001 - MC-011 constructs
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKHH

SEQ ID NO: 66 - DNA, 1500 bp, Artificial Sequence
  DNA Sequences for the MC-001 - MC-011 constructs
atgcaggccaaaaatgatattaccctggaagatctgccgtatctgatcaaaaaaatcgatcagaacgaactggaagccgatattggtgatattaccgcactggaaaaatatctggcactgagccagtatggaaatattctggccctggaagaactgaataaagctctggaagagctggatgaagatgtgggttggaatcagaatgatatcgccaatctggaagatgatgttgaaaccctgaccaaaaatcagaatgcactggcagaacagggtgaagcaattaaagaagatctgcagggtctggcagattttgttgaaggtcaggaaggcaaaattctgcagaacgaaaccagcatcaaaaaaaacacccagcgtaatctggtgaatggctttgaaattgaaaaaaacaaagatgccattgccaaaaacaacgaaagcattgaagatctgtatgattttggtcatgaagttgccgaaagcattggtgaaattcatgcacataacgaagcacagaatgaaaccctgaaaggtctgattaccaacagcatcgaaaataccaataacattaccaaaaacaaagcagatattcaggcgctggaaaataatgttgtggaagaactgtttaatctgagcggtcgtctgattgatcagaaagccgatatcgataataacattaacaacatttatgaactggcacagcagcaggatcagcatagcagcgatatcaaaaccctgaaaaaaaacgttgaagaaggtctgctggaactgtctggtcacctgatcgatcagaaaactgatattgcccagaatcaggcaaatattcaggatctggccacctataatgaactgcaggatcagtatgcacagaaacagaccgaagcaattgatgccctgaataaagcgagcagcgaaaacacccagaatatcgaagatctggcagcatacaacgaactgcaggatgcctatgcaaaacagcagactgaagccatcgacgcactgaacaaggcaagctctgaaaacacgcagaacattgaagatctggctgcctataatgaattacaggatgcgtatgccaaacagcagaccgaagcgattgatgcgctgaacaaagcctcttctgaaaatacacagaatatcgccaaaaatcaggccgatattgccaacaatatcaataatatctatgaactggcccagcagcaggatcagcactcttctgatatcaaaacactggcaaaagcaagcgcagcaaataccgatcgtattgcgaaaaacaaagccgatgcagatgcaagctttgaaacactgacgaaaaaccagaacaccctgattgaaaaagataaagaacatgataaactgatcaccgccaataaaaccgcaattgatgcaaataaagccagcgcagataccaaatttgcagcaaccgcagatgcaattaccaaaaatggcaatgccatcaccaaaaatgccaaaagcgcaagccatcatcatcaccaccactaa

SEQ ID NO: 67 - protein, 499 aa, Artificial Sequence
  Protein Sequences for the MC-001 - MC-011 constructs
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSASHHHHHH

SEQ ID NO: 68 - DNA, 1476 bp, Artificial Sequence
  DNA Sequences for the MC-001 - MC-011 constructs
atgcaggccaaaaatgatattaccctggaagatctgccgtatctgatcaaaaaaatcgatcagaacgaactggaagccgatattggtgatattaccgcactggaaaaatatctggcactgagccagtatggaaatattctggccctggaagaactgaataaagctctggaagagctggatgaagatgtgggttggaatcagaatgatatcgccaatctggaagatgatgttgaaaccctgaccaaaaatcagaatgcactggcagaacagggtgaagcaattaaagaagatctgcagggtctggcagattttgttgaaggtcaggaaggcaaaattctgcagaacgaaaccagcatcaaaaaaaacacccagcgtaatctggtgaatggctttgaaattgaaaaaaacaaagatgccattgccaaaaacaacgaaagcattgaagatctgtatgattttggtcatgaagttgccgaaagcattggtgaaattcatgcacataacgaagcacagaatgaaaccctgaaaggtctgattaccaacagcatcgaaaataccaataacattaccaaaaacaaagcagatattcaggcgctggaaaataatgttgtggaagaactgtttaatctgagcggtcgtctgattgatcagaaagccgatatcgataataacattaacaacatttatgaactggcacagcagcaggatcagcatagcagcgatatcaaaaccctgaaaaaaaacgttgaagaaggtctgctggaactgtctggtcacctgatcgatcagaaaactgatattgcccagaatcaggcaaatattcaggatctggccacctataatgaactgcaggatcagtatgcacagaaacagaccgaagcaattgatgccctgaataaagcgagcagcgaaaacacccagaatatcgaagatctggcagcatacaacgaactgcaggatgcctatgcaaaacagcagactgaagccatcgacgcactgaacaaggcaagctctgaaaacacgcagaacattgaagatctggctgcctataatgaattacaggatgcgtatgccaaacagcagaccgaagcgattgatgcgctgaacaaagcctcttctgaaaatacacagaatatcgccaaaaatcaggccgatattgccaacaatatcaataatatctatgaactggcccagcagcaggatcagcactcttctgatatcaaaacactggcaaaagcaagcgcagcaaataccgatcgtattgcgaaaaacaaagccgatgcagatgcaagctttgaaacactgacgaaaaaccagaacaccctgattgaaaaagataaagaacatgataaactgatcaccgccaataaaaccgcaattgatgcaaataaagccagcgcagataccaaatttgcagcaaccgcagatgcaattaccaaaaatggcaatgccatcaccaaaaatgccaaaagctaa

SEQ ID NO: 69 - protein, 491 aa, Artificial Sequence
  Protein Sequences for the MC-001 - MC-011 constructs
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKS

SEQ ID NO: 70 - DNA, 1635 bp, Artificial Sequence
  DNA Sequences for the MC-001 - MC-011 constructs
atgcaggccaaaaatgatattaccctggaagatctgccgtatctgatcaaaaaaatcgatcagaacgaactggaagccgatattggtgatattaccgcactggaaaaatatctggcactgagccagtatggaaatattctggccctggaagaactgaataaagctctggaagagctggatgaagatgtgggttggaatcagaatgatatcgccaatctggaagatgatgttgaaaccctgaccaaaaatcagaatgcactggcagaacagggtgaagcaattaaagaagatctgcagggtctggcagattttgttgaaggtcaggaaggcaaaattctgcagaacgaaaccagcatcaaaaaaaacacccagcgtaatctggtgaatggctttgaaattgaaaaaaacaaagatgccattgccaaaaacaacgaaagcattgaagatctgtatgattttggtcatgaagttgccgaaagcattggtgaaattcatgcacataacgaagcacagaatgaaaccctgaaaggtctgattaccaacagcatcgaaaataccaataacattaccaaaaacaaagcagatattcaggcgctggaaaataatgttgtggaagaactgtttaatctgagcggtcgtctgattgatcagaaagccgatatcgataataacattaacaacatttatgaactggcacagcagcaggatcagcatagcagcgatatcaaaaccctgaaaaaaaacgttgaagaaggtctgctggaactgtctggtcacctgatcgatcagaaaactgatattgcccagaatcaggcaaatattcaggatctggccacctataatgaactgcaggatcagtatgcacagaaacagaccgaagcaattgatgccctgaataaagcgagcagcgaaaacacccagaatatcgaagatctggcagcatacaacgaactgcaggatgcctatgcaaaacagcagactgaagccatcgacgcactgaacaaggcaagctctgaaaacacgcagaacattgaagatctggctgcctataatgaattacaggatgcgtatgccaaacagcagaccgaagcgattgatgcgctgaacaaagcctcttctgaaaatacacagaatatcgccaaaaatcaggccgatattgccaacaatatcaataatatctatgaactggcccagcagcaggatcagcactcttctgatatcaaaacactggcaaaagcaagcgcagcaaataccgatcgtattgcgaaaaacaaagccgatgcagatgcaagctttgaaacactgacgaaaaaccagaacaccctgattgaaaaagataaagaacatgataaactgatcaccgccaataaaaccgcaattgatgcaaataaagccagcgcagataccaaatttgcagcaaccgcagatgcaattaccaaaaatggcaatgccatcaccaaaaatgccaaaagcattaccgatctgggcaccaaagttgatggttttgatagccgtgtgaccgcactggataccaaagttaatgcatttgatggtcgtattaccgctctggatagtaaagttgaaaatggtatggcagcacaggcagcagcaagccatcatcatcaccaccactaa

SEQ ID NO: 71 - protein, 544 aa, Artificial Sequence
  Protein Sequences for the MC-001 - MC-011 constructs
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAASHHHHHH

SEQ ID NO: 72 - DNA, 1617 bp, Artificial Sequence
  DNA Sequences for the MC-001 - MC-011 constructs
atgcaggccaaaaatgatattaccctggaagatctgccgtatctgatcaaaaaaatcgatcagaacgaactggaagccgatattggtgatattaccgcactggaaaaatatctggcactgagccagtatggaaatattctggccctggaagaactgaataaagctctggaagagctggatgaagatgtgggttggaatcagaatgatatcgccaatctggaagatgatgttgaaaccctgaccaaaaatcagaatgcactggcagaacagggtgaagcaattaaagaagatctgcagggtctggcagattttgttgaaggtcaggaaggcaaaattctgcagaacgaaaccagcatcaaaaaaaacacccagcgtaatctggtgaatggctttgaaattgaaaaaaacaaagatgccattgccaaaaacaacgaaagcattgaagatctgtatgattttggtcatgaagttgccgaaagcattggtgaaattcatgcacataacgaagcacagaatgaaaccctgaaaggtctgattaccaacagcatcgaaaataccaataacattaccaaaaacaaagcagatattcaggcgctggaaaataatgttgtggaagaactgtttaatctgagcggtcgtctgattgatcagaaagccgatatcgataataacattaacaacatttatgaactggcacagcagcaggatcagcatagcagcgatatcaaaaccctgaaaaaaaacgttgaagaaggtctgctggaactgtctggtcacctgatcgatcagaaaactgatattgcccagaatcaggcaaatattcaggatctggccacctataatgaactgcaggatcagtatgcacagaaacagaccgaagcaattgatgccctgaataaagcgagcagcgaaaacacccagaatatcgaagatctggcagcatacaacgaactgcaggatgcctatgcaaaacagcagactgaagccatcgacgcactgaacaaggcaagctctgaaaacacgcagaacattgaagatctggctgcctataatgaattacaggatgcgtatgccaaacagcagaccgaagcgattgatgcgctgaacaaagcctcttctgaaaatacacagaatatcgccaaaaatcaggccgatattgccaacaatatcaataatatctatgaactggcccagcagcaggatcagcactcttctgatatcaaaacactggcaaaagcaagcgcagcaaataccgatcgtattgcgaaaaacaaagccgatgcagatgcaagctttgaaacactgacgaaaaaccagaacaccctgattgaaaaagataaagaacatgataaactgatcaccgccaataaaaccgcaattgatgcaaataaagccagcgcagataccaaatttgcagcaaccgcagatgcaattaccaaaaatggcaatgccatcaccaaaaatgccaaaagcattaccgatctgggcaccaaagttgatggttttgatagccgtgtgaccgcactggataccaaagttaatgcatttgatggtcgtattaccgctctggatagtaaagttgaaaatggtatggcagcacaggcagcacaccactaa

SEQ ID NO: 73 - protein, 538 aa, Artificial Sequence
  Protein Sequences for the MC-001 - MC-011 constructs
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 74 - DNA, 1614 bp, Artificial Sequence
  DNA Sequences for the MC-001 - MC-011 constructs
atggcgaaaaatgatattaccctggaagatctgccgtatctgatcaaaaaaatcgatcagaacgaactggaagccgatattggtgatattaccgcactggaaaaatatctggcactgagccagtatggaaatattctggccctggaagaactgaataaagctctggaagagctggatgaagatgtgggttggaatcagaatgatatcgccaatctggaagatgatgttgaaaccctgaccaaaaatcagaatgcactggcagaacagggtgaagcaattaaagaagatctgcagggtctggcagattttgttgaaggtcaggaaggcaaaattctgcagaacgaaaccagcatcaaaaaaaacacccagcgtaatctggtgaatggctttgaaattgaaaaaaacaaagatgccattgccaaaaacaacgaaagcattgaagatctgtatgattttggtcatgaagttgccgaaagcattggtgaaattcatgcacataacgaagcacagaatgaaaccctgaaaggtctgattaccaacagcatcgaaaataccaataacattaccaaaaacaaagcagatattcaggcgctggaaaataatgttgtggaagaactgtttaatctgagcggtcgtctgattgatcagaaagccgatatcgataataacattaacaacatttatgaactggcacagcagcaggatcagcatagcagcgatatcaaaaccctgaaaaaaaacgttgaagaaggtctgctggaactgtctggtcacctgatcgatcagaaaactgatattgcccagaatcaggcaaatattcaggatctggccacctataatgaactgcaggatcagtatgcacagaaacagaccgaagcaattgatgccctgaataaagcgagcagcgaaaacacccagaatatcgaagatctggcagcatacaacgaactgcaggatgcctatgcaaaacagcagactgaagccatcgacgcactgaacaaggcaagctctgaaaacacgcagaacattgaagatctggctgcctataatgaattacaggatgcgtatgccaaacagcagaccgaagcgattgatgcgctgaacaaagcctcttctgaaaatacacagaatatcgccaaaaatcaggccgatattgccaacaatatcaataatatctatgaactggcccagcagcaggatcagcactcttctgatatcaaaacactggcaaaagcaagcgcagcaaataccgatcgtattgcgaaaaacaaagccgatgcagatgcaagctttgaaacactgacgaaaaaccagaacaccctgattgaaaaagataaagaacatgataaactgatcaccgccaataaaaccgcaattgatgcaaataaagccagcgcagataccaaatttgcagcaaccgcagatgcaattaccaaaaatggcaatgccatcaccaaaaatgccaaaagcattaccgatctgggcaccaaagttgatggttttgatagccgtgtgaccgcactggataccaaagttaatgcatttgatggtcgtattaccgctctggatagtaaagttgaaaatggtatggcagcacaggcagcacaccactaa

SEQ ID NO: 75 - protein, 537 aa, Artificial Sequence
  Protein Sequences for the MC-001 - MC-011 constructs
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 76 - DNA, 1611 bp, Artificial Sequence
  DNA Sequences for the MC-001 - MC-011 constructs
atgcaggccaaaaatgatattaccctggaagatctgccgtatctgatcaaaaaaatcgatcagaacgaactggaagccgatattggtgatattaccgcactggaaaaatatctggcactgagccagtatggaaatattctggccctggaagaactgaataaagctctggaagagctggatgaagatgtgggttggaatcagaatgatatcgccaatctggaagatgatgttgaaaccctgaccaaaaatcagaatgcactggcagaacagggtgaagcaattaaagaagatctgcagggtctggcagattttgttgaaggtcaggaaggcaaaattctgcagaacgaaaccagcatcaaaaaaaacacccagcgtaatctggtgaatggctttgaaattgaaaaaaacaaagatgccattgccaaaaacaacgaaagcattgaagatctgtatgattttggtcatgaagttgccgaaagcattggtgaaattcatgcacataacgaagcacagaatgaaaccctgaaaggtctgattaccaacagcatcgaaaataccaataacattaccaaaaacaaagcagatattcaggcgctggaaaataatgttgtggaagaactgtttaatctgagcggtcgtctgattgatcagaaagccgatatcgataataacattaacaacatttatgaactggcacagcagcaggatcagcatagcagcgatatcaaaaccctgaaaaaaaacgttgaagaaggtctgctggaactgtctggtcacctgatcgatcagaaaactgatattgcccagaatcaggcaaatattcaggatctggccacctataatgaactgcaggatcagtatgcacagaaacagaccgaagcaattgatgccctgaataaagcgagcagcgaaaacacccagaatatcgaagatctggcagcatacaacgaactgcaggatgcctatgcaaaacagcagactgaagccatcgacgcactgaacaaggcaagctctgaaaacacgcagaacattgaagatctggctgcctataatgaattacaggatgcgtatgccaaacagcagaccgaagcgattgatgcgctgaacaaagcctcttctgaaaatacacagaatatcgccaaaaatcaggccgatattgccaacaatatcaataatatctatgaactggcccagcagcaggatcagcactcttctgatatcaaaacactggcaaaagcaagcgcagcaaataccgatcgtattgcgaaaaacaaagccgatgcagatgcaagctttgaaacactgacgaaaaaccagaacaccctgattgaaaaagataaagaacatgataaactgatcaccgccaataaaaccgcaattgatgcaaataaagccagcgcagataccaaatttgcagcaaccgcagatgcaattaccaaaaatggcaatgccatcaccaaaaatgccaaaagcattaccgatctgggcaccaaagttgatggttttgatagccgtgtgaccgcactggataccaaagttaatgcatttgatggtcgtattaccgctctggatagtaaagttgaaaatggtatggcagcacaggcagcataa

SEQ ID NO: 77 - protein, 536 aa, Artificial Sequence
  Protein Sequences for the MC-001 - MC-011 constructs
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAA

SEQ ID NO: 78 - DNA, 1560 bp, Artificial Sequence
  DNA Sequences for the MC-001 - MC-011 constructs
atggcgaaaaatgatattaccctggaagatctgccgtatctgatcaaaaaaatcgatcagaacgaactggaagccgatattggtgatattaccgcactggaaaaatatctggcactgagccagtatggaaatattctggccctggaagaactgaataaagctctggaagagctggatgaagatgtgggttggaatcagaatgatatcgccaatctggaagatgatgttgaaaccctgaccaaaaatcagaatgcactggcagaacagggtgaagcaattaaagaagatctgcagggtctggcagattttgttgaaggtcaggaaggcaaaattctgcagaacgaaaccagcatcaaaaaaaacacccagcgtaatctggtgaatggctttgaaattgaaaaaaacaaagatgccattgccaaaaacaacgaaagcattgaagatctgtatgattttggtcatgaagttgccgaaagcattggtgaaattcatgcacataacgaagcacagaatgaaaccctgaaaggtctgattaccaacagcatcgaaaataccaataacattaccaaaaacaaagcagatattcaggcgctggaaaataatgttgtggaagaactgtttaatctgagcggtcgtctgattgatcagaaagccgatatcgataataacattaacaacatttatgaactggcacagcagcaggatcagcatagcagcgatatcaaaaccctgaaaaaaaacgttgaagaaggtctgctggaactgtctggtcacctgatcgatcagaaaactgatattgcccagaatcaggcaaatattcaggatctggccacctataatgaactgcaggatcagtatgcacagaaacagaccgaagcaattgatgccctgaataaagcgagcagcgaaaacacccagaatatcgaagatctggcagcatacaacgaactgcaggatgcctatgcaaaacagcagactgaagccatcgacgcactgaacaaggcaagctctgaaaacacgcagaacattgaagatctggctgcctataatgaattacaggatgcgtatgccaaacagcagaccgaagcgattgatgcgctgaacaaagcctcttctgaaaatacacagaatatcgccaaaaatcaggccgatattgccaacaatatcaataatatctatgaactggcccagcagcaggatcagcactcttctgatatcaaaacactggcaaaagcaagcgcagcaaataccgatcgtattgcgaaaaacaaagccgatgcagatgcaagctttgaaacactgacgaaaaaccagaacaccctgattgaaaaagataaagaacatgataaactgatcaccgccaataaaaccgcaattgatgcaaataaagccagcgcagataccaaatttgcagcaaccgcagatgcaattaccaaaaatggcaatgccatcaccaaaaatgccaaaagcattaccgatctgggcaccaaagttgatggttttgatagccgtgtgaccgcactggataccaaagcaagccatcatcatcaccaccactaa

SEQ ID NO: 79 - protein, 519 aa, Artificial Sequence
  Protein Sequences for the MC-001 - MC-011 constructs
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKASHHHHHH

SEQ ID NO: 80 - DNA, 1893 bp, Moraxella catarrhalis
atgaaaaccatgaaacttctccctctaaaaatcgctgtaaccagtgccatgattattggcttgggtgcggcatctactgcgaatgcgcaggctaaaaatgatataactttagaggatttaccatatttaataaaaaagattgaccaaaatgaattggaagcagatatcggagatattactgctcttgaaaagtatctagcacttagccagtatggcaatattttagctctagaagagctcaacaaggctctagaagagctcgacgaggatgttggatggaatcagaatgatattgcaaacttggaagatgatgttgaaacgctcaccaaaaatcaaaatgctttggctgaacaaggtgaggcaattaaagaagatcttcaagggcttgcagattttgtagaagggcaagagggtaaaattctacaaaatgaaacttcaattaaaaaaaatactcagagaaaccttgtcaatgggtttgagattgagaaaaataaagatgctattgctaaaaacaatgagtctatcgaagatctttatgattttggtcatgaggttgcagaaagtataggcgagatacatgctcataatgaagcgcaaaatgaaactcttaaaggcttgataacaaacagtattgagaatactaataatattaccaaaaacaaagctgacatccaagcacttgaaaacaatgtcgtagaagaactattcaatctaagcggtcgcctaattgatcaaaaagcagatattgataataacatcaacaatatctatgagctggcacaacagcaagatcagcatagctctgatatcaaaacacttaaaaaaaatgtcgaagaaggtttgttggagctaagcggtcacctaattgatcaaaaaacagatattgctcaaaaccaagctaacatccaagatctggccacttacaacgagctacaagaccagtatgctcaaaagcaaaccgaagcgattgacgctctaaataaagcaagctctgagaatacacaaaacatcgaagatctggccgcttacaacgagctacaagatgcctatgccaaacagcaaaccgaagcaattgacgctctaaataaagcaagctctgagaatacacaaaacatcgaagatctggccgcttacaacgagctacaagatgcctatgccaaacagcaaaccgaagccattgacgctctaaataaagcaagctctgagaatacacaaaacattgctaaaaaccaagcggatattgctaataacatcaacaatatctatgagctggcacaacagcaagatcagcatagctctgatatcaaaaccttggcaaaagcaagtgctgccaatactgatcgtattgctaaaaacaaagccgatgctgatgcaagttttgaaacgctcaccaaaaatcaaaatactttgattgaaaaagataaagagcatgacaaattaattactgcaaacaaaactgcgattgatgccaataaagcatctgcggataccaagtttgcagcgacagcagacgccattaccaaaaatggaaatgctatcactaaaaacgcaaaatctatcactgatttgggcactaaagtggatggttttgacagtcgtgtaactgcattagacaccaaagtcaatgcctttgatggtcgtatcacagctttagacagtaaagttgaaaacggtatggctgcccaagctgccctaagtggtctattccagccttatagcgttggtaagtttaatgcgaccgctgcacttggtggctatggctcaaaatctgcggttgctatcggtgctggctatcgtgtgaatccaaatctggcgtttaaagctggtgcggcgattaataccagtggtaataaaaaaggctcttataacatcggtgtgaattacgagttctaa

SEQ ID NO: 81 - protein, 52 aa, Moraxella catarrhalis
AATADAITKNGNAITKNAKSITDLGTKVDGFDSRVTALDTKVNAFDGRITAL

SEQ ID NO: 82 - protein, 14 aa, Moraxella catarrhalis
  MISC_FEATURE (7)..(7): Xaa can be Ala or Gln
  MISC_FEATURE (10)..(11): XaaXaa can be GlnLys or LysGln
YNELQDXYAXXQTE

SEQ ID NO: 83 - protein, 14 aa, Moraxella catarrhalis
YNELQDAYAQKQTE

SEQ ID NO: 84 - protein, 14 aa, Moraxella catarrhalis
YNELQDAYAKQQTE

SEQ ID NO: 85 - protein, 14 aa, Moraxella catarrhalis
YNELQDQYAQKQTE

SEQ ID NO: 86 - protein, 14 aa, Moraxella catarrhalis
YNELQDQYAKQQTE

SEQ ID NO: 87 - protein, 5 aa, Artificial Sequence
  Antibody VH Chain CDRs
SYWMH

SEQ ID NO: 88 - protein, 17 aa, Artificial Sequence
  Antibody VH Chain CDRs
AIYPGNSDTSYNQKFKG

SEQ ID NO: 89 - protein, 7 aa, Artificial Sequence
  Antibody VH Chain CDRs
LRFLDAY

SEQ ID NO: 90 - protein, 10 aa, Artificial Sequence
  Antibody VL Chain CDRs
RASSSVSYMH

SEQ ID NO: 91 - protein, 7 aa, Artificial Sequence
  Antibody VL Chain CDRs
ATSNLAS

SEQ ID NO: 92 - protein, 9 aa, Artificial Sequence
  Antibody VL Chain CDRs
QQWSSNPPT

SEQ ID NO: 93 - DNA, 349 bp, Artificial Sequence
  Antibody VH Chain DNA
gaggttcagctccagcagtctgggactgtgctggcaaggcctggggcttccgtgaagatgtcctgcaaggcttctggctacagctttaccagctactggatgcactgggtaaaacagaggcctggacagggtctagaatggattggtgctatttatcctggaaatagtgatactagctacaaccagaagttcaagggcaaggccaaactgactgcagtcacatccgccagcactgcctacatggagctcagcaacctgacgaatgaggactctgcggtctattcctgtacattactacgtttcctcgatgcttactggggccaagggactctggtcactgtctctgcag

SEQ ID NO: 94 - protein, 116 aa, Artificial Sequence
  Antibody VH Chain Protein
EVQLQQSGTVLARPGASVKMSCKASGYSFTSYWMHWVKQRPGQGLEWIGAIYPGNSDTSYNQKFKGKAKLTAVTSASTAYMELSNLTNEDSAVYSCTLLRFLDAYWGQGTLVTVSA

SEQ ID NO: 95 - DNA, 318 bp, Artificial Sequence
  Antibody VL Chain DNA
caaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagtagtaacccacccacgttcggaggggggtccaagctggaaataaaa

SEQ ID NO: 96 - protein, 106 aa, Artificial Sequence
  Antibody VL Chain Protein
QIVLSQSPAILSASPGEKVTMTCRASSSVSYMHWYQQKPGSSPKPWIYATSNLASGVPARFSGSGSGTSYSLTISRVEAEDAATYYCQQWSSNPPTFGGGSKLEIK

SEQ ID NO: 97 - protein, 8 aa, Artificial Sequence
  Tag residues
ASHHHHHH

SEQ ID NO: 98 - DNA, 21 bp, Artificial Sequence
  Primers
gcaaggcttacaaccacaatc

SEQ ID NO: 99 - DNA, 21 bp, Artificial Sequence
  Primers
gaggacagggcttgattgtgg

SEQ ID NO: 100 - DNA, 21 bp, Artificial Sequence
  Primers
gaggacaggggttgattgttg

SEQ ID NO: 101 - DNA, 22 bp, Artificial Sequence
  Primers
ggacaggggttctgtgttatgg

SEQ ID NO: 102 - DNA, 20 bp, Artificial Sequence
  Primers
ctgggcttgggtattctagg

SEQ ID NO: 103 - DNA, 22 bp, Artificial Sequence
  Primers
ctcattcctgttgaagctcttg

SEQ ID NO: 104 - DNA, 20 bp, Artificial Sequence
  Primers
gcacgggacaaactcttctc

SEQ ID NO: 105 - DNA, 22 bp, Artificial Sequence
  Primers
ctgcaggagacagactcttctc

SEQ ID NO: 106 - DNA, 54 bp, Artificial Sequence
  Primers
gtctcgtgggctcggagatgtgtataagagacaggttagtttgggcagcagatc

SEQ ID NO: 107 - DNA, 53 bp, Artificial Sequence
  Primers
gtctcgtgggctcggagatgtgtataagagacaggaggagccagttgtayctc

SEQ ID NO: 108 - DNA, 55 bp, Artificial Sequence
  Primers
gtctcgtgggctcggagatgtgtataagagacaggaaccagttgtatctccacac

SEQ ID NO: 109 - DNA, 53 bp, Artificial Sequence
  Primers
gtctcgtgggctcggagatgtgtataagagacaggatccagatgtgtcactgc

SEQ ID NO: 110 - DNA, 55 bp, Artificial Sequence
  Primers
gtctcgtgggctcggagatgtgtataagagacaggtgggaagatggatacagttg

SEQ ID NO: 111 - DNA, 54 bp, Artificial Sequence
  Primers
gtctcgtgggctcggagatgtgtataagagacaggagctcttcagaggaaggtg

SEQ ID NO: 112 - DNA, 53 bp, Artificial Sequence
  Primers
gtctcgtgggctcggagatgtgtataagagacagctcagrggaaggtggaaac

SEQ ID NO: 113 - DNA, 50 bp, Artificial Sequence
  Primers
tcgtcggcagcgtcagatgtgtataagagacagttttttttttttttttt

SEQ ID NO: 114 - DNA, 16 bp, Artificial Sequence
  Primers
gtaaaacgacggccag

SEQ ID NO: 115 - DNA, 17 bp, Artificial Sequence
  Primers
caggaaacagctatgac

SEQ ID NO: 116 - protein, 7 aa, Moraxella catarrhalis
DTKVNAF

SEQ ID NO: 117 - protein, 7 aa, Moraxella catarrhalis
DGRITAL

SEQ ID NO: 118 - protein, 37 aa, Moraxella catarrhalis
ATADAITKNGNAITKNAKSITDLGTKVDGFDSRVTAL

SEQ ID NO: 119 - protein, 19 aa, Moraxella catarrhalis
YNELQDQYAQKQTEAIDAL

SEQ ID NO: 120 - protein, 18 aa, Moraxella catarrhalis
AIDALNKASSENTQNIED

SEQ ID NO: 121 - protein, 15 aa, Moraxella catarrhalis
QDAYAKQQTEAIDAL

SEQ ID NO: 122 - protein, 60 aa, Moraxella catarrhalis
KVNAFDGRITALDSKVENGMAAQAALSGLFQPYSVGKFNATAALGGYGSKSAVAIGAGYR

SEQ ID NO: 123 - protein, 60 aa, Yersinia enterocolitica
KFRQLDNRLDKLDTRVDKGLASSAALNSLFQPYGVGKVNFTAGVGGTRSSQALAIGSGYR

SEQ ID NO: 124 - protein, 31 aa, Moraxella catarrhalis
VNPNLAFKAGAAINTSGNKKGSYNIGVNYEF

SEQ ID NO: 125 - protein, 31 aa, Yersinia enterocolitica
  MISC_FEATURE (14)..(14): Xaa can be any amino acids
  MISC_FEATURE (15)..(15): Xaa can be any amino acids
  MISC_FEATURE (15)..(15): Xaa can be any amino acid
VNESVALKAGVAYXXAGSSDVMYNASFNIEW